(19) United States
(12) Patent Application Publication
(10) Pub. No.: US 2019/0085404 A1
(43) Pub. Date: Mar. 21, 2019

(54) METHODS OF IDENTIFYING AND TREATING IMMUNE CHECKPOINT INHIBITOR-RESPONSIVE NEOPLASMS AND CELLS, TRANSGENIC ANIMALS AND KITS FOR USE THEREIN

(71) Applicant: The Board of Trustees of the Leland Stanford Junior University, Stanford, CA (US)

(72) Inventors: Dean W. Felsher, San Mateo, CA (US); Stephanie C. Casey Parks, Redwood City, CA (US)

(21) Appl. No.: 16/080,980

(22) PCT Filed: Mar. 3, 2017
(86) PCT No.: PCT/US2017/020799

§ 371 Date: Aug. 29, 2018

Related U.S. Application Data
(60) Provisional application No. 62/303,950, filed on Mar. 4, 2016.

(51) Int. Cl. C12Q 1/6886; A61K 31/551; G01N 33/574; G01N 33/50; A01K 67/027

(52) U.S. Cl. CPC C12Q 1/6886; A61K 31/551; G01N 33/5748; G01N 33/5011; A01K 2227/105; C12Q 2600/106; C12Q 2600/158; G01N 2800/52; A01K 2267/0331; A01K 67/0275

(57) ABSTRACT

Provided are methods of identifying whether a subject having cancer will be responsive to agents that combat immune evasion, such as immune checkpoint inhibitors. Methods of treating a subject having cancer are also provided. Such methods may include those that involve identifying whether the cancer will be responsive to an immune checkpoint inhibitor and/or is an immune-evasive cancer and administering an agent, e.g., an immune checkpoint inhibitor and/or a MYC inhibitor, to the subject to treat the cancer. Also provided are methods of identifying cancer therapeutics that are effective during MYC-regulated immune evasion as well as cancer cell lines and transgenic animals useful in such methods. Kits for use in the described methods are also provided.

### CROSS-REFERENCE TO RELATED APPLICATIONS

Pursuant to 35 U.S.C. § 119(e), this application claims priority to the filing date of the U.S. Provisional Patent Application Ser. No. 62/303,950, filed Mar. 4, 2016, the disclosure of which application is herein incorporated by reference.

### GOVERNMENT RIGHTS

This invention was made with Government support under contracts R01 CA 089305, R01 CA170378, R01 CA184384, CA 188383, CA 114747, CA 105102, CA 112973, P01 CA034233, 1F32CA177139 and 5T32AI07290 awarded by the National Institutes of Health. The Government has certain rights in the invention.

### BACKGROUND

Cancer remains one of the leading causes of death globally, with an estimated 12.7 million cases around the world affecting both sexes equally. This number is expected to increase to 21 million by 2030 (see e.g., Vinay et al. Semin Cancer Biol. 2015; 35 Suppl:S185-98). Thus, cancer remains a significant public health burden around the globe.

The immune system interacts intimately with tumors over the entire process of disease development and progression to metastasis. This complex cross talk between immunity and cancer cells can both inhibit and enhance tumor growth. As such, immune system involvement is now classified as a hallmark of cancer (see e.g., Hanahan & Weinberg. Cell, 144 (2011), pp. 646-674). In many circumstances, the immune system is able to recognize and mitigate cancer producing cells, made possible by complex molecular processes that coordinate cancer recognition and immune checkpoints. Immune cells are activated to kill cancer cells while the overall immune response is tempered to suppress unnecessary cytotoxic effects on non-cancer cells. Advances in cancer immunotherapy have aided and expanded upon this natural capability of the immune system, in some cases successfully combating cancer progression.

However, a significant number of different types of human tumors can suppress the host immune system to enhance their survival. In addition, cancer immune evasion has become a major obstacle in developing and deploying cutting-edge cancer therapies, particularly in the field of immuno-oncology where agents are specifically designed to target immune molecules which cancers evolve to circumvent. As such, with or without ongoing therapy, many human cancers evade the host immune system.

### SUMMARY

Provided are methods of identifying whether a subject having cancer will be responsive to agents that combat immune evasion, such as immune checkpoint inhibitors. Methods of treating a subject having cancer are also provided. Such methods may include those that involve identifying whether the cancer will be responsive to an immune checkpoint inhibitor and/or is an immune-evasive cancer and administering an agent, e.g., an immune checkpoint inhibitor and/or a MYC inhibitor, to the subject to treat the cancer. Also provided are methods of identifying cancer therapeutics that are effective during MYC-regulated immune evasion as well as cancer cell lines and transgenic animals useful in such methods. Kits for use in the described methods are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1C demonstrate that MYC regulates the expression of CD47 and PD-L1 in murine and human leukemia and lymphomas.

FIG. 2A-2C demonstrate that MYC regulates CD47 and PD-L1 expression in human and mouse tumors and binds to the promoters of the corresponding genes.

FIG. 3A-3B demonstrate that constitutive expression of CD47 and PD-L1 in mouse MYC T-ALL 4188 cells prevents recruitment of immune effectors after MYC inactivation.

FIG. 4A-4F demonstrate that down-regulation of CD47 or PD-L1 is required for tumor regression, shutdown of angiogenesis, and induction of senescence upon MYC inactivation.

FIG. 5A-5B depict that CD47 and PD-L1 are two important immune regulatory molecules often expressed on human tumors and provide immunofluorescence showing that MYC inactivation resulted in a rapid downregulation of CD47 and PD-L1.

FIG. 6A-6B demonstrate that a MYC-targeting shRNA or JQ1 both reduced the expression of MYC mRNA in human T-ALL cell lines and primary human T-ALL cells.

FIG. 7A-7E demonstrate that CD47 and PD-L1 were either unaffected or showed increased expression upon treatment of MYC TALL cells with chemotherapeutic drugs and that the chemotherapeutic drugs has no effect on CD3, CD8, CD25, and CD69 expression.

FIG. 8A-8C demonstrate that MYC regulates the mRNA and protein expression of CD47 and PD-L1 in hepatocellular carcinoma (HCC).

FIG. 9A-9B demonstrate that MYC regulates expression of CD47 and PD-L1 in primary human T-ALL.

FIG. 10A-10B demonstrate that MYC expression correlates with CD47 and PD-L1 (CD274) expression in human tumors.

FIG. 11 provides MYC DNA binding for various immune related genes in conditional transgenic mouse MYC T-ALL cells.

FIG. 12 provides MYC DNA binding for various immune related genes in human P493-6 Burkitt lymphoma-like cells.

FIG. 13 provides MYC DNA binding for PD-L1 and CD47 in conditional MYC-driven U2OS cells.

FIG. 14A-14B depict MYC induced expression of the CD47 gene along with other well-known target genes in a nuclear run-on assay.

FIG. 15 demonstrates a lack of increased protein turnover of CD47 or PD-L1 proteins compared to other immune surface proteins in mouse MYC T-ALL cells when protein synthesis was inhibited.

FIG. 16A-16B demonstrate that, in MYC T-ALL 4188 cells constitutively overexpressing CD47 or PD-L1, CD47 and PD-L1 mRNA levels were unaffected by MYC inactivation.

FIG. 17 demonstrates that sustained expression of CD47 or PD-L1 inhibits the recruitment of immune cells following MYC inactivation.

FIG. 18A-18B demonstrate that the shRNA mediated reduced expression of CD47 or PD-L1 prevents tumor engraftment.

FIG. 19A-19D demonstrate that sustained expression of CD47 or PD-L1 alters angiogenesis and senescence.

FIG. 20A-20E demonstrate that sustained expression of CD47 or PD-L1 does not affect apoptosis or proliferation arrest upon MYC inactivation.

FIG. 21 provides a schematic representation demonstrating the regulation of immunological checkpoints in MYC-driven tumors.

FIG. 22 provides the structures of various examples of small molecule direct and indirect MYC inhibitors.

FIG. 23 provides the structures of various examples of small molecules that bind c-Myc promoter G-quadruplexes.

FIG. 24 provides the structures of various examples of small molecule PD-L1/PD1 pathway inhibitors.

FIG. 25 provides the structures of examples of peptidomimetic inhibitors of the PD-1/PD-L1 interaction.

FIG. 26 provides an example of a structure of a macrocyclic peptidic PD1/PD-L1 inhibitor.

FIG. 27 provides an example of a structure of a peptide antagonists of PD-L1.

### DEFINITIONS

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻³ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “antibody” and “immunoglobulin”, as used herein, are used interchangeably may generally refer to whole or intact molecules or fragments thereof and modified and/or conjugated antibodies or fragments thereof that have been modified and/or conjugated. The immunoglobulins can be divided into five different classes, based on differences in the amino acid sequences in the constant region of the heavy chains. All immunoglobulins within a given class will have very similar heavy chain constant regions. These differences can be detected by sequence studies or more commonly by serological means (i.e. by the use of antibodies directed to these differences). Immunoglobulin classes include IgG (Gamma heavy chains), IgM (Mu heavy chains), IgA (Alpha heavy chains), IgD (Delta heavy chains), and IgE (Epsilon heavy chains).

Antibody or immunoglobulin may refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated as V_{H}) and a heavy chain constant region (abbreviated as C_{H}). The heavy chain constant region typically is comprised of three domains, C_{H}1, C_{H}2, and C_{H}3. Each light chain typically is comprised of a light chain variable region (abbreviated as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Whole or largely intact antibodies are generally multivalent, meaning they may simultaneously bind more than one molecule of antigen whereas antibody fragments may be monovalent. Antibodies produced by an organism as part of an immune response are generally monospecific, meaning they generally bind a single species of antigen. Multivalent monospecific antibodies, i.e. antibodies that bind more than one molecule of a single species of antigen, may bind a single antigen epitope (e.g., a monoclonal antibody) or multiple different antigen epitopes (e.g., a polyclonal antibody).

Multispecific (e.g., bispecific) antibodies, which bind multiple species of antigen, may be readily engineered by those of ordinary skill in the art and, thus, may be encompassed within the use of the term “antibody” used herein where appropriate. Also, multivalent antibody fragments may be engineered, e.g., by the linking of two monovalent antibody fragments. As such, bivalent and/or multivalent antibody fragments may be encompassed within the use of the term “antibody”, where appropriate, as the ordinary skilled artisan will be readily aware of antibody fragments, e.g., those described below, which may be linked in any convenient and appropriate combination to generate multivalent monospecific or polyspecific (e.g., bispecific) antibody fragments.

Antibody fragments include but are not limited to antigen-binding fragments (Fab or F(ab), including Fab′ or F(ab′), (Fab)₂, F(ab′)₂, etc.), single chain variable fragments (scFv or Fv), “third generation” (3G) molecules, etc. which are capable of binding the epitopic determinant. These antibody fragments retain some ability to selectively bind to the subject antigen, examples of which include, but are not limited to:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab)₂, the fragment of the antibody that can be obtained by treating whole antibody with the enzyme pepsin without subsequent reduction;

(4) F(ab)₂ is a dimer of two Fab′ fragments held together by two disulfide bonds;

(5) Fv, defined as a genetically engineered fragment containing the variable region of the light chain and the variable region of the heavy chain expressed as two chains;

(6) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, tetrabodies, etc. which may or may not be polyspecific (see, for example, WO 94/07921 and WO 98/44001) and

(7) “3G”, including single domain (typically a variable heavy domain devoid of a light chain) and “miniaturized” antibody molecules (typically a full-sized Ab or mAb in which non-essential domains have been removed).

The term “small molecule” is used to refer to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, heterocyclic rings, etc.). In some embodiments, small molecules are monomeric and have a molecular weight of less than about 1500 g/mol. In certain embodiments, the molecular weight of the small molecule is less than about 1000 g/mol or less than about 500 g/mol. In certain embodiments, small molecules are biologically active in that they produce a biological effect in animals, e.g., mammals, e.g., humans.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with cancer; those suspected of having cancer; etc.).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The terms “co-administration” and “in combination with” include the administration of two or more therapeutic agents either simultaneously, concurrently or sequentially within no specific time limits. In one embodiment, the agents are present in the cell or in the subjects body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent that inhibits a target gene (e.g., a MYC-dependent target gene, and the like) and/or compositions is an amount that is sufficient, when administered to the individual, to palliate, ameliorate, stabilize, reverse, prevent, slow or delay the progression of the disease state (e.g., cancer, etc.) by, for example, inhibiting the growth of, inducing death of or otherwise preventing the clinical progressing of a MYC-dependent cancer present in the subject.

### DETAILED DESCRIPTION

Provided are methods of identifying whether a subject having cancer will be responsive to agents that combat immune evasion, such as immune checkpoint inhibitors. Methods of treating a subject having cancer are also provided. Such methods may include those that involve identifying whether the cancer will be responsive to an immune checkpoint inhibitor and/or is an immune-evasive cancer and administering an agent, e.g., an immune checkpoint inhibitor and/or a MYC inhibitor, to the subject to treat the cancer. Also provided are methods of identifying cancer therapeutics that are effective during MYC-regulated immune evasion as well as cancer cell lines and transgenic animals useful in such methods. Kits for use in the described methods are also provided.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed

### Methods

As summarized above, methods are provided for identifying whether a subject having a neoplasm will be responsive to agents that combat immune evasion, such as immune checkpoint inhibitors. Such methods may include determining whether a cellular sample from the neoplasm overexpresses a MYC oncogene, where the overexpression of the MYC oncogene in the sample is indicative that the neoplasm is an immune checkpoint inhibitor responsive neoplasm. Such methods may include identifying a subject as responsive (or likely to be responsive) to treatment with an immune checkpoint inhibitor based on detected over expression of MYC within a cellular sample of a neoplasm from the subject. Similarly, the methods may include identifying a subject as non-responsive (or unlikely to be responsive) to treatment with an immune checkpoint inhibitor based on a detected level of MYC expression within a cellular sample of a neoplasm from the subject that does not amount to over expression.

By “overexpresses a MYC oncogene” is generally meant that a cell or a population of cells expresses MYC above a known (i.e., predetermined) level, e.g., threshold level or a normal level. MYC overexpression may include expression of a MYC encoding mRNA above a predetermined level, expression of a MYC protein above a predetermined level or a combination thereof. A predetermined level of MYC, above which a cell or a population of cells may be identified as overexpressing MYC, includes e.g., a normal level of MYC expression.

Predetermined and/or normal levels of MYC expression may be obtained through a variety of means. For example, in some instances, a predetermined level of MYC expression may be obtained from a cell known not to overexpress MYC, whether or not the cell is neoplastic, or from a population of cells known not to overexpress MYC, whether or not the population is neoplastic. In some instances, a predetermined level of MYC expression may be obtained from a cell known to overexpress MYC, whether or not the cell is neoplastic, or from a population of cells known to overexpress MYC, whether or not the population is neoplastic. Cells known to overexpress MYC or known not to overexpress MYC may include, e.g., those cells where MYC expression is controlled by an inducible and/or conditional expression system and the system is in an “ON” or “OFF” state, respectively. For example, a level of MYC expression measured from a cell know not to overexpress MYC, such as e.g., a cell with inducible/conditional MYC expression where MYC is off, may be used to establish a threshold above which a cell is considered to overexpress MYC. In some instances, a level of MYC expression measured from a cell know to overexpress MYC, such as e.g., a cell with inducible/conditional MYC expression where MYC is on, may be used to establish a threshold below which a cell is considered not to overexpress MYC.

In some instances, a predetermined or a normal level of MYC expression may be obtained from a cell known not to be a neoplastic cell or from a population of cells known not to be a neoplastic population of cells. For example, in some instances, a cell expressing a normal level of MYC or a population of cells expressing a normal level of MYC may include a non-neoplastic cell that does not overexpress MYC or a population of non-neoplastic cells that does not overexpress MYC. In some instances, a cell expressing a normal level of MYC or a population of cells expressing a normal level of MYC may include a neoplastic cell that does not overexpress MYC or a population of neoplastic cells that does not overexpress MYC. Normal levels of MYC expression, whether in neoplastic or non-neoplastic cells, may also be referred to as baseline expression levels. Neoplastic cells, and populations thereof, that do not overexpress MYC include but are not limited to e.g., neoplastic cells obtained from cancers not driven by MYC (i.e., non-MYC driven cancers); neoplastic cells where MYC, MYC activity and/or MYC signaling has or has not been disrupted; and the like. Cells with known and/or predetermined levels of MYC expression, including normal levels and overexpression levels, may be referred to as a control or control cells (e.g., including positive and negative controls), where control cells may, but need not necessarily, be the same or an equivalent cell type to cells under examination to which they are compared.

Levels of MYC expression in cells that do not overexpress MYC may be employed to establish a threshold for MYC expression above which a cell or a population of cells may be identified as “MYC overexpressing”. Such thresholds may be absolute or relative. Non-limiting examples of relative MYC expression thresholds above which a cell or a population of cells may be identified as overexpressing MYC include 1.1 times normal or control MYC expression or greater, including e.g., 1.2 times or greater, 1.3 times or greater, 1.4 times or greater, 1.5 times or greater, 1.6 times or greater, 1.7 times or greater, 1.8 times or greater, 1.9 times or greater, 2.0 times or greater, 2.1 times or greater, 2.2 times or greater, 2.3 times or greater, 2.4 times or greater, 2.5 times or greater, 2.6 times or greater, 2.7 times or greater, 2.8 times or greater, 2.9 times or greater, 3.0 times or greater, 3.5 times or greater, 4 times or greater, 4.5 times or greater, 5 times or greater, 6 times or greater, 7 times or greater, 8 times or greater, 9 times or greater, 10 times or greater, etc.

Levels of MYC expression in cells that overexpress MYC may be employed to establish a threshold for MYC expression below which a cell or a population of cells may be identified as not MYC overexpressing. Such thresholds may be absolute or relative. Non-limiting examples of relative MYC expression thresholds below which a cell or a population of cells may be identified as not overexpressing MYC include 0.9 times the level of MYC overexpression or less, including e.g., 0.8 times or less, 0.7 times or less, 0.6 times or less, 0.5 times or less, 0.4 times or less, 0.3 times or less, 0.2 times or less, 0.1 times or less, etc.

Relative levels of MYC expression, including protein levels and/or mRNA levels, may be determined through the use of various qualitative or quantitative methods including but not limited to e.g., differential expression microarray analysis, semi-quantitative PCR, quantitative PCR (qPCR), next generation sequencing (NGS), immuno-assays, fluorescence activated cell sorting (FACS), mass spectrometry, and the like. Absolute levels of MYC expression, including protein and/or mRNA, may be determined through the use of various quantitative methods including but not limited to e.g., qPCR, quantitative NGS, quantitative mass spectrometry, and the like.

Accordingly, the provided methods may include measuring the level of expression of a MYC oncogene in a cellular sample obtained from a subject and/or measuring the level of expression of a MYC oncogene in a cell of the cellular sample, wherein the measuring may be performed by any convenient method of measuring expression. Useful methods of measuring MYC oncogene expression include methods of measuring MYC encoding mRNA and/or MYC protein levels including but not limited to e.g., those methods of measuring mRNA and/or protein levels described above.

Determining the level of expression of MYC may also, in some instance, include measuring the level of expression of one or more MYC target genes. For example, in some instances, MYC overexpression may be inferred from the level of expression, overexpression or elevated expression of a MYC target gene. By “MYC target gene” is meant a gene, the expression of which is regulated (induced or inhibited) by the MYC transcription factor. Non-limiting examples of MYC target genes include PDK1, CHEK1, CDK2, LDHA, and ODC1.

MYC (also referred to as “Myc oncogene protein” or “Myc proto-oncogene protein”) is a transcription factor encoded by the c-Myc gene that regulates the expression of a multitude of gene products involved in cell proliferation, growth, differentiation, and apoptosis. The c-Myc gene is genetically activated and overexpressed in many cancers and this overexpression has been causally linked to tumorigenesis.

In humans, c-Myc (NCBI Gene ID: 4609) is present on chromosome 8 at 8q24.21. Human MYC protein (GenBank Accession No. NP_002458.2; UniProt P01106) has the following amino acid sequence:

(SEQ ID NO: 1)

MDFFRVVENQQPPATMPLNVSFTNRNYDLDYDSVQPYFYCDEEENFYQQQ
QQSELQPPAPSEDIWKKFELLPTPPLSPSRRSGLCSPSYVAVTPFSLRGD
NDGGGGSFSTADQLEMVTELLGGDMVNQSFICDPDDETFIKNIIIQDCMW
SGFSAAAKLVSEKLASYQAARKDSGSPNPARGHSVCSTSSLYLQDLSAAA
SECIDPSVVFPYPLNDSSSPKSCASQDSSAFSPSSDSLLSSTESSPQGSP
EPLVLHEETPPTTSSDSEEEQEDEEEIDVVSVEKRQAPGKRSESGSPSAG
GHSKPPHSPLVLKRCHVSTHQHNYAAPPSTRKDYPAAKRVKLDSVRVLRQ
ISNNRKCTSPRSSDTEENVKRRTHNVLERQRRNELKRSFFALRDQIPELE
NNEKAPKVVILKKATAYILSVQAEEQKLISEEDLLRKRREQLKHKLEQLR
NSCA.

In mice, c-Myc (NCBI Gene ID: 17869) is present on chromosome 15 at 15 D1 (15 26.19 cM). Mouse MYC protein (GenBank Accession Nos. NP_001170823.1, NP_001170824.1, NP_001170825.1, NP_034979.3; UniProt P01108) has the following amino acid sequence:

(SEQ ID NO: 2)

MPLNVNFTNRNYDLDYDSVQPYFICDEEENFYHQQQQSELQPPAPSEDIW
KKFELLPTPPLSPSRRSGLCSPSYVAVATSFSPREDDDGGGGNFSTADQL
EMMTELLGGDMVNQSFICDPDDETFIKNIIIQDCMWSGFSAAAKLVSEKL
ASYQAARKDSTSLSPARGHSVCSTSSLYLQDLTAAASECIDPSVVFPYPL
NDSSSPKSCTSSDSTAFSPSSDSLLSSESSPRASPEPLVLHEETPPTTSS
DSEEEQEDEEEIDVVSVEKRQTPAKRSESGSSPSRGHSKPPHSPLVLKRC
HVSTHQHNYAAPPSTRKDYPAAKRAKLDSGRVLKQISNNRKCSSPRSSDT
EENDKRRTHNVLERQRRNELKRSFFALRDQIPELENNEKAPKVVILKKAT
AYILSIQADEHKLTSEKDLLRKRREQLKHKLEQLRNSGA.

In rats (Rattus norvegicus), c-Myc (NCBI Gene ID: 24577) is present on chromosome 7 at 7q33. Rat MYC protein (GenBank Accession No. NP_036735.2; UniProt P09416) has the following amino acid sequence:

(SEQ ID NO: 3)

MNFLWEVENPTVTTMPLNVSFANRNYDLDYDSVQPYFICDEEENFYHQQQ
QSELQPPAPSEDIWKKFELLPTPPLSPSRRSGLCSPSYVAVATSFSPRED
DDGGGGNFSTADQLEMMTELLGGDMVNQSFICDPDDETFIKNIIIQDCMW
SGFSAAAKLVSEKLASYQAARKDSTSLSPARGHSVCSTSSLYLQDLTAAA
SECIDPSVVFPYPLNDSSSPKSCTSSDSTAFSSSSDSLLSSESSPRATPE
PLVLHEETPPTTSSDSEEEQDDEEEIDVVSVEKRQPPAKRSESGSSPSRG
HSKPPHSPLVLKRCHVSTHQHNYAAPPSTRKDYPAAKRAKLDSGRVLKQI
SNNRKCSSPRSSDTEENDKRRTHNVLERQRRNELKRSFFALRDQIPELEN
NEKAPKVVILKKATAYILSVQADEHKLISEKDLLRKRREQLKHKLEQLRN
SGA.

As summarized above, the provided methods may identify whether a subject having a neoplasia will be responsive to an immune checkpoint inhibitor based on whether a sample of the neoplasia overexpresses MYC. Without being bound by theory, the subject method takes advantage of the discovery that MYC regulates the expression of immune checkpoint proteins in cancer cells. MYC expression was found to be directly correlated with the expression of immune checkpoint genes such that MYC overexpression resulted in high levels of expression of immune checkpoint proteins.

By “immune checkpoint protein” is generally meant a protein that regulates one or more immune cell stimulatory or inhibitory pathways. Inhibitory immune checkpoint proteins prevent activation of the immune cell in response to, e.g., an antigen expressed on the target cell. Immune checkpoints are crucial for maintaining self-tolerance and modulating the duration and amplitude of physiological immune responses in order to minimize collateral tissue damage. Inhibitory immune checkpoint proteins may be expressed by cancer cells to evade recognition and/or destruction by the host immune system. Accordingly, inhibitory immune checkpoint proteins may be expressed on the surface of a cell to render the cell capable of evading the host immune system, termed “immune evasion”. For example, a cancer cell may express an inhibitory immune checkpoint protein, thus signaling to a host immune cell through the immune checkpoint protein thereby repressing immune recognition, activation of the immune cell and/or phagocytosis.

Examples of inhibitory immune checkpoint protein ligands (and their relevant immune cell receptor) include but are not limited to e.g., PD-L1 (PD1); PD-L2 (PD1); CD80 (CLTA4, also known as CD152); CD86 (CLTA4); GAL9 (TIM3); HVEM (BTLA, also known as CD272); B7-H3, also known as CD276; B7-H4, also known as VTCN1; CD47, also known as IAP (SIRPα); CD200, also known as OX-2 (CD200R); and the like. Such inhibitory immune checkpoint proteins are described in e.g., Pardoll. Nat Rev Cancer. 2012; 12(4):252-64; Liu et al. Nat Med. 2015; 21(10):1209-15; Liu et al. J Hematol Oncol. 2017; 10(1):12; Śledzińska et al. Mol Oncol. 2015; 9(10):1936-65; the disclosures of which are incorporated by reference herein in their entirety.

In the provided methods a determination that a cellular sample, or a cell therefrom, overexpresses a MYC oncogene indicates that the subject is likely to respond to treatment with an inhibitor of an immune checkpoint protein. For example, in some instances, a determination that a cellular sample, or a cell therefrom, overexpresses a MYC oncogene indicates that the subject is likely to respond to an inhibitor of the immune checkpoint protein CD47 or an agent that inhibits the expression of CD47. In some instances, a determination that a cellular sample, or a cell therefrom, overexpresses a MYC oncogene indicates that the subject is likely to respond to an inhibitor of the immune checkpoint protein PD-L1 or an agent that inhibits the expression of PD-L1.

Accordingly, in some embodiments, the provided methods may include administering to a subject an effective amount of one or more immune checkpoint inhibitors when the subject is identified as having a neoplasm that overexpresses MYC. In some embodiments, following a determination that the subject harbors a neoplasm that overexpresses MYC, the subject may be administered an effective amount of a CD47 inhibitor. In some embodiments, following a determination that the subject harbors a neoplasm that overexpresses MYC, the subject may be administered an effective amount of a PD-L1 inhibitor. In some embodiments, following a determination that the subject harbors a neoplasm that overexpresses MYC, the subject may be administered effective amounts of two or more immune checkpoint inhibitors. In some embodiments, following a determination that the subject harbors a neoplasm that overexpresses MYC, the subject may be administered effective amounts of both a CD47 inhibitor and a PD-L1 inhibitor. Useful immune checkpoint inhibitors are described greater detail below.

In some instances, where a cellular sample, or a cell therefrom, from a subject's neoplasm is determined not to overexpress MYC the subject may not be administered an immune checkpoint inhibitor or further evaluation may be indicated before the subject is administered an immune checkpoint inhibitor. For example, where a cellular sample of a neoplasm from a subject is determined to not overexpress MYC the sample and/or the neoplasm may be subjected to further testing to determine if the subject is likely or unlikely to be responsive to the immune checkpoint inhibitor. In some instances, a cellular sample found to not overexpress MYC may be further tested for expression of one or more immune checkpoint proteins (e.g., PD-L1, CD47, etc.) prior to administering an immune checkpoint inhibitor (e.g., a PD-L1 inhibitor, a CD47 inhibitor, etc.) to the subject. In some embodiments, if the non-MYC-overexpressing neoplasm from the subject is determined to express an inhibitory immune checkpoint protein, only then will the subject be administered an immune checkpoint inhibitor directed at the expressed inhibitory immune checkpoint protein. In instances, where a non-MYC-overexpressing neoplasm from the subject is determined to not express an inhibitory immune checkpoint protein, then other treatment options may be explored, e.g., conventional cancer therapy (e.g., surgery, radiation therapy, chemotherapy, etc.) or immunotherapy directed to targets other than inhibitory immune checkpoint proteins (e.g., adoptive T cell immunotherapy, etc.).

The provided methods may find use with subjects having a variety of different neoplasms, including but not limited to cancers. Relevant cancers include tumors (e.g., solid tumors (e.g., sarcomas and carcinomas) and blood cancers. Non-limited examples of various cancers to which the subject methods may be applied include: Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

In some instances, a subject to which the provided methods may be applied may be a subject having a hematological (i.e., blood) cancer, e.g., a leukemia or a lymphoma. Non-limiting examples of hematological cancers include: Acute Lymphoblastic Leukemia, Adult; Acute Lymphoblastic Leukemia, Childhood; Acute Myeloid Leukemia, Adult; Acute Myeloid Leukemia, Childhood; Chronic Lymphocytic Leukemia; Chronic Myelogenous Leukemia; Hairy Cell Leukemia; AIDS-Related Lymphoma; Cutaneous T-Cell Lymphoma (see Mycosis Fungoides and the Sézary Syndrome); Hodgkin Lymphoma, Adult; Hodgkin Lymphoma, Childhood; Hodgkin Lymphoma During Pregnancy; Mycosis Fungoides; Non-Hodgkin Lymphoma, Adult; Non-Hodgkin Lymphoma, Childhood; Non-Hodgkin Lymphoma During Pregnancy; Primary Central Nervous System Lymphoma; Sézary Syndrome; T-Cell Lymphoma, Cutaneous (see Mycosis Fungoides and the Sézary Syndrome); Waldenström Macroglobulinemia (see Non-Hodgkin Lymphoma) Chronic Myeloproliferative Neoplasms; Langerhans Cell Histiocytosis; Multiple Myeloma/Plasma Cell Neoplasm; Myelodysplastic Syndromes; Myelodysplastic/Myeloproliferative Neoplasms; and the like.

In some instances, a subject to which the provided methods may be applied may be a subject having a carcinoma (e.g., an adenocarcinoma or a squamous cell carcinoma). Non-limiting examples of carcinomas include: acinar carcinoma, acinic cell carcinoma, acinous carcinoma, adenocystic carcinoma, adenoid cystic carcinoma, adenosquamous carcinoma, adnexal carcinoma, adrenocortical carcinoma, alveolar carcinoma, ameloblastic carcinoma, apocrine carcinoma, basal cell carcinoma, bronchioloalveolar carcinoma, bronchogenic carcinoma, cholangiocellular carcinoma, chorionic carcinoma, clear cell carcinoma, colloid carcinoma, colorectral carcinoma, cribriform carcinoma, ductal carcinoma in situ, embryonal carcinoma, carcinoma en cuirasse, endometrioid carcinoma, epidermoid carcinoma, carcinoma ex mixed tumor, carcinoma ex pleomorphic adenoma, follicular carcinoma of thyroid gland, hepatocellular carcinoma, carcinoma in si'tu, intraductal carcinoma, Hürthle cell carcinoma, inflammatory carcinoma of the breast, large cell carcinoma, invasive lobular carcinoma, lobular carcinoma, lobular carcinoma in situ (LCIS), medullary carcinoma, meningeal carcinoma, Merkel cell carcinoma, mucinous carcinoma, mucoepidermoid carcinoma, nasopharyngeal carcinoma, non-small cell carcinoma, non-small cell lung carcinoma (NSCLC), oat cell carcinoma, papillary carcinoma, renal cell carcinoma, scirrhous carcinoma, sebaceous carcinoma, carcinoma sim'plex, signet-ring cell carcinoma, small cell carcinoma, small cell lung carcinoma, spindle cell carcinoma, squamous cell carcinoma, terminal duct carcinoma, transitional cell carcinoma, tubular carcinoma, verrucous carcinoma, and the like.

In some instances, a subject to which the provided methods may be applied may be a subject having a melanoma. Non-limiting examples of melanomas include: cutaneous melanoma (e.g., superficial spreading melanoma, nodular melanoma, acral lentiginous melanoma, lentigo maligna melanoma), mucosal melanoma and ocular melanoma.

The provided methods may include obtaining a cellular sample of a neoplasia from the subject, including e.g., where the neoplasia is a cancer (e.g., a blood cancer, a carcinoma, a melanoma, etc.), including a cancer described above. Any convenient means of obtaining a cellular sample may be employed. For example, where the neoplasia is a blood cancer the cellular sample may be obtained by collecting a blood sample from the subject. Peripheral blood samples may or may not be fractionated to isolated specific cell populations, e.g., white blood cells. In another example, where the neoplasia is solid tumor the cellular sample may be obtained through a biopsy of the solid tumor.

A method of measuring MYC expression may be applied directly to the obtained cellular sample or the cellular sample may be prepared in some manner prior to the MYC expression measurement. In some embodiments, a sample may be prepared for quantitative or qualitative assessment of MYC expression by contacting the sample with a reagent that specifically binds MYC protein (e.g., an anti-MYC antibody) or a reagent that specifically binds a MYC encoding nucleic acid (e.g., a nucleic acid probe that specifically hybridizes to a MYC encoding mRNA). Such reagents may or may not be detectably labeled. For example, in some instances, the cellular sample may be applied to a slide and MYC expression may be evaluated by staining the slide with one or more reagents specific for MYC protein or MYC encoding mRNA. Staining of a cellular sample applied to a slide may make use of one or more histological techniques such as tissue embedding/freezing, sectioning (e.g., plastic sectioning, cryosectioning, etc.) fixation, permeabilization, histological staining, immunostaining/immunofluorescence (i.e., antibody staining), and the like. Staining of a cellular sample to detect expression of MYC encoding nucleic acids may make use of a variety of nucleic acid detection techniques such as fixation, permeabilization, in situ hybridization (e.g., fluorescent in situ hybridization (FISH)), and the like.

Cellular samples need not necessarily be applied to a slide and/or stained for evaluation of MYC expression. For example, in some instances, liquid based and/or cytological techniques may be employed including e.g., cytometry (e.g., flow cytometry, fluorescence activated cell sorting (FACS), mass cytometry (CyTOF), and the like), well-based cell sorting, microfluidics, and the like, and the cellular sample may be appropriately prepared using e.g., fixation, permeabilization, tissue dissociation, staining, cell suspension preparation, etc. In some instances, molecular techniques (e.g., microarray, PCR, sequencing, mass spectrometry, enzyme-linked immunosorbent assay (ELISA), Western Blot, etc.) may be employed for evaluation of MYC expression and the cellular sample may be correspondingly prepared, e.g., using tissue/cell dissociation, cell lysis, nucleic acid purification/isolation/extraction (e.g., solid or liquid phase), protein purification/isolation/extraction (e.g., solid or liquid phase), cDNA preparation, nucleic acid amplification, nucleic acid labeling, protein labeling, etc.

In embodiments where a MYC encoding nucleic acid is detected and/or quantified through amplification, the sample may be contacted with one or more nucleic acid primers (e.g., two nucleic acid primers) that specifically hybridize to a MYC encoding mRNA or a produced MYC cDNA (e.g., produced in a reverse transcription (RT) reaction, such as e.g., RT-PCR). Accordingly, in some instances, detection of MYC overexpression and/or measurement of MYC expression may include the amplification of a MYC encoding nucleic acid using one or more MYC-specific nucleic acid primers.

In some instances, preparation of the sample may include contacting the sample with one or more detectably labeled probes (e.g., antibody probes, nucleic acid probes, etc.) for MYC. Detectably labeled probes include e.g., alkaline phosphatase conjugated probes, horseradish peroxidase conjugated probes, fluorophore-conjugates probes (e.g., probes conjugated to a fluorophore such as e.g., Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 650/665, BODIPY TMR-X, BODIPY TR-X, Dialkylaminocoumarin, Cy5.5, Cy5, Cy3.5, Cy3, etc.) and the like.

Methods and reagents for use in processing of cellular samples for MYC expression analysis may, but need not necessarily, include those methods and reagents described in Li et al., Am J Clin Pathol. 2015; 143(4):598-604; Oberly et al., Histopathology. 2013; 63(4):499-508; Mahmoud et al. Mod Pathol. 2015; 28(4):545-51; as well as US Patent Application Pub. Nos: 20170029904, 20170016074, 20160177396, 20150240315, 20150197819, 20150080269, 20120270914; the disclosures of which are incorporated herein by reference in their entirety.

In some embodiments, MYC expression levels may be employed in stratifying subjects for clinical trials. For example, in some instances, MYC expression levels may be employed in stratifying subject for clinical trials involving the use of an immune checkpoint inhibitor. According to some embodiments, subjects in a clinical trial testing an immune checkpoint inhibitor may be stratified based on whether the subjects overexpress MYC. In some instances, MYC expression may be determined for a cellular sample of a neoplasia from a subject for a clinical trial to detect whether the subject overexpresses MYC and the subject may be included or excluded from the trial based on the determined MYC expression. For example, in some instances, subjects overexpressing MYC or subjects expressing MYC at various levels may be included and/or stratified in a clinical trial testing a PD-L1 inhibitor. In some instances, subjects overexpressing MYC or subjects expressing MYC at various levels may be included and/or stratified in a clinical trial testing a CD47 inhibitor.

As summarized above, the provided methods include methods of treating a subject for a neoplasia. Thus, an individual to be treated according to the present methods will generally be an individual with a neoplasia. As used herein “neoplasia” includes any form of abnormal new tissue formation; and the like. In some cases, the individual has recently undergone treatment for neoplasia (e.g., cancer, a tumor, etc.) and are therefore at risk for recurrence. In some instances, the individual has not recently or previously undergone treatment for a neoplasia (e.g., cancer, a tumor, etc.) but has been newly diagnosed with a neoplasia. Any and all neoplasia are suitable neoplasia to be treated by the subject methods e.g., utilizing one or more therapeutic agents, e.g., as described below, or a herein described kit.

Subjects that may be treated according to the described methods include subject that have previously undergone or are currently undergoing treatment for a neoplasia. Such subjects may display symptoms of cancer reoccurrence or regrowth of a neoplasia. Such subject may also have been nonresponsive to the prior or current therapy. In some instances, cancer reoccurrence and/or neoplasia regrowth and/or treatment non-responsiveness may be a result of immune evasion of the cancer or neoplasia. In some instances, the prior therapy to which a subject may have been non-responsive or from which the cancer/neoplasia has reoccurred/regrown may have been a conventional cancer therapy (e.g., surgery, radiation therapy, chemotherapy, etc.). In some instances, the subject may have been non-responsive to prior or concurrent chemotherapy, including but not limited to e.g., prior chemotherapy involving one or more of prednisone, cytoxan, cisplatin, vincristine and the like.

In some embodiments, the methods of treatment include identifying whether the neoplasia will be responsive to an immune checkpoint inhibitor (e.g., according to the methods of detecting MYC overexpression as described above) and administering an effective amount of an immune checkpoint inhibitor when the neoplasia is identified as responsive or likely to be responsive (e.g., is identified as over expressing MYC).

In some embodiments, the provided methods of treatment include treating a subject having a MYC-regulated immune-evasive cancer by administering to the subject a therapeutic agent that is effective against the MYC-regulated immune-evasive cancer. In some instances, the methods may include determining whether a cellular sample of an immune-evasive cancer obtained from a subject overexpresses an immune checkpoint protein (e.g., CD47, PD-L1 or both) and administering an effective amount of an agent to the subject to treat the immune-evasive cancer. In some instances, the methods may include determining whether a cellular sample of an neoplasm obtained from a subject overexpresses an immune checkpoint protein (e.g., CD47, PD-L1 or both) and MYC and administering an effective amount of an agent to the subject to treat the neoplasm.

As described above, without being bound by theory, the subject method takes advantage of the discovery that MYC regulates the expression of immune checkpoint proteins in cancer cells. Thus, detection of overexpression of one or more immune checkpoint proteins may be indicative of MYC misregulation (e.g., overexpression). Accordingly, the provided methods include treating a subject for a neoplasia identified as overexpressing an immune checkpoint protein by administering to the subject an effective amount of a MYC inhibitor. In some instances, the overexpressed immune checkpoint protein may be CD47. In some instances, the overexpressed immune checkpoint protein may be PD-L1. In some instances, the subject may overexpress two or more immune checkpoint proteins, including but not limited to e.g., both CD47 and PD-L1.

In some embodiments, the methods of treating may involve administering to a subject having a neoplasia that overexpresses an immune checkpoint protein a therapeutic agent that is not an immune checkpoint inhibitor to treat the subject for the neoplasia. Such agents include agents that do not directly target an immune checkpoint protein or the immune cell receptor thereof. An example of a therapeutic agent that is not an immune checkpoint inhibitor but may be employed in the methods of treating a subject having a neoplasia that overexpresses one or more immune checkpoint proteins is a MYC inhibitor. However, such therapeutic agents that are not immune checkpoint inhibitors are not limited to MYC and may include e.g., therapeutic agents identified that are effective during MYC-regulated immune evasion but do not directly target an immune checkpoint protein, including e.g., agents uncovered utilizing the screening methods described below.

Accordingly, in some embodiments, the provided methods may include determining whether a cellular sample of a neoplasm obtained from a subject overexpresses an immune checkpoint protein. As summarized herein, overexpression of an immune checkpoint protein in a cellular sample of the neoplasm may be indicative of the subject being responsive to a therapeutic agent, such as an agent other than an inhibitor of the immune check point protein, e.g., a MYC inhibitor or other non-immune-checkpoint-inhibitor agent.

Such methods may include identifying a subject as responsive (or likely to be responsive) to treatment with a therapeutic agent other than an immune checkpoint inhibitor (e.g., a MYC inhibitor) based on detected over expression of the immune checkpoint inhibitor within a cellular sample of a neoplasm from the subject. Similarly, the methods may include identifying a subject as non-responsive (or unlikely to be responsive) to treatment with a therapeutic agent other than an immune checkpoint inhibitor (e.g., a MYC inhibitor) based on a detected level of expression of the immune checkpoint inhibitor within a cellular sample of a neoplasm from the subject that does not amount to over expression.

By “overexpresses an immune checkpoint protein” is generally meant that a cell or a population of cells expresses an immune checkpoint protein above a known (i.e., predetermined) level, e.g., threshold level or a normal level. Immune checkpoint protein overexpression may include expression of an immune checkpoint protein encoding mRNA above a predetermined level, expression of an immune checkpoint protein above a predetermined level or a combination thereof. A predetermined level of an immune checkpoint protein, above which a cell or a population of cells may be identified as overexpressing the immune checkpoint protein, includes e.g., a normal level of expression of the immune checkpoint protein, a minimal detectable level or the like.

Predetermined, normal and/or minimal detectable levels of immune checkpoint protein expression may be obtained through a variety of means. In some instances, a predetermined or a normal level of immune checkpoint protein expression may be obtained from a cell known not to be a neoplastic cell or from a population of cells known not to be a neoplastic population of cells. In some instances, a predetermined or a normal level of immune checkpoint protein expression may be obtained from a cell known not to be an immune evasive cell (e.g., known not to be an immune evasive neoplastic cell) or from a population of cells known not to be an immune evasive population of cells (e.g., known not to be an immune evasive population of neoplastic cells). Normal and/or minimal levels of immune checkpoint protein expression, whether in neoplastic or non-neoplastic cells, may also be referred to as baseline expression levels. Neoplastic cells, and populations thereof, that do not overexpress immune checkpoint protein include but are not limited to e.g., neoplastic cells obtained from cancers that are not immune evasive; neoplastic cells where inhibitory immune checkpoint protein encoding gene(s) have been disrupted; and the like. Cells with known and/or predetermined levels of immune checkpoint protein expression, including normal levels and overexpression levels, may be referred to as a control or control cells (e.g., including positive and negative controls), where control cells may, but need not necessarily, be the same or an equivalent cell type to cells under examination to which they are compared.

Levels of immune checkpoint protein expression in cells that do not overexpress the immune checkpoint protein may be employed to establish a threshold for immune checkpoint protein expression above which a cell or a population of cells may be identified as “immune checkpoint protein overexpressing”. Such thresholds may be absolute or relative. Non-limiting examples of relative immune checkpoint protein expression thresholds above which a cell or a population of cells may be identified as overexpressing an immune checkpoint protein include 1.1 times baseline or control immune checkpoint protein expression or greater, including e.g., 1.2 times or greater, 1.3 times or greater, 1.4 times or greater, 1.5 times or greater, 1.6 times or greater, 1.7 times or greater, 1.8 times or greater, 1.9 times or greater, 2.0 times or greater, 2.1 times or greater, 2.2 times or greater, 2.3 times or greater, 2.4 times or greater, 2.5 times or greater, 2.6 times or greater, 2.7 times or greater, 2.8 times or greater, 2.9 times or greater, 3.0 times or greater, 3.5 times or greater, 4 times or greater, 4.5 times or greater, 5 times or greater, 6 times or greater, 7 times or greater, 8 times or greater, 9 times or greater, 10 times or greater, etc.

Levels of immune checkpoint protein expression in cells that overexpress an immune checkpoint protein may be employed to establish a threshold for immune checkpoint protein expression below which a cell or a population of cells may be identified as not immune checkpoint protein-overexpressing. Such thresholds may be absolute or relative. Non-limiting examples of relative immune checkpoint protein expression thresholds below which a cell or a population of cells may be identified as not overexpressing an immune checkpoint protein include 0.9 times the level of immune checkpoint protein overexpression or less, including e.g., 0.8 times or less, 0.7 times or less, 0.6 times or less, 0.5 times or less, 0.4 times or less, 0.3 times or less, 0.2 times or less, 0.1 times or less, etc.

Relative levels of immune checkpoint protein expression, including protein levels and/or mRNA levels, may be determined through the use of various qualitative or quantitative methods including but not limited to e.g., differential expression microarray analysis, semi-quantitative PCR, quantitative PCR (qPCR), next generation sequencing (NGS), immuno-assays, fluorescence activated cell sorting (FACS), mass cytometry (CyTOF), mass spectrometry, and the like. Absolute levels of immune checkpoint protein expression, including protein and/or mRNA, may be determined through the use of various quantitative methods including but not limited to e.g., qPCR, quantitative NGS, quantitative mass spectrometry, and the like.

Accordingly, the provided methods may include measuring the level of expression of an immune checkpoint protein in a cellular sample obtained from a subject and/or measuring the level of expression of an immune checkpoint protein in a cell of the cellular sample, wherein the measuring may be performed by any convenient method of measuring expression. Useful methods of measuring an immune checkpoint protein expression include methods of measuring an immune checkpoint protein encoding mRNA and/or an immune checkpoint protein levels including but not limited to e.g., those methods of measuring mRNA and/or protein levels described above, including those employing techniques and reagents described in relation to measuring MYC expression.

In some instances, the provided methods may include measuring the level and/or detecting overexpression of the immune checkpoint protein PD-L1. In humans, PD-L1 (also known as CD274, B7-H, B7H1, B7-H and B7H1) is encoded by the PD-L1/CD274 gene (NCBI Gene ID: 29126) present on chromosome 9 at 9p24.1. PD-L1 is immune inhibitory receptor ligand that is expressed by hematopoietic and non-hematopoietic cells, such as T cells and B cells and various types of tumor cells. The encoded protein is a type I transmembrane protein that has immunoglobulin V-like and C-like domains. Interaction of this ligand with its receptor inhibits T-cell activation and cytokine production. During infection or inflammation of normal tissue, this interaction is important for preventing autoimmunity by maintaining homeostasis of the immune response. In tumor microenvironments, this interaction provides an immune escape for tumor cells through cytotoxic T-cell inactivation.

Human PD-L1 protein (GenBank Accession No. NP_001254635.1, NP_001300958.1, NP_054862.1; UniProt Q9NZQ7) has the following three isoform amino acid sequences:

### Isoform 1:

(SEQ ID NO: 4)

MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET;

### Isoform 2:

(SEQ ID NO: 5)

MRIFAVFIFMTYWHLLNAPYNKINQRILVVDPVTSEHELTCQAEGYPKAE
VIWTSSDHQVLSGKTTTTNSKREEKLFNVTSTLRINTTTNEIFYCTFRRL
DPEENHTAELVIPELPLAHPPNERTHLVILGAILLCLGVALTFIFRLRKG
RMMDVKKCGIQDTNSKKQSDTHLEET;

### Isoform 3:

(SEQ ID NO: 6)

MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGD.

In mice, PD-L1 is encoded by the CD274 gene (also known as B7h1, Pdl1, Pdcd1l1, Pdcd1lg1 and A530045L16Rik; NCBI Gene ID: 60533) present on chromosome 19 at 19 C1. Mouse PD-L1 protein (GenBank Accession No. NP_068693.1; UniProt Q9EP73) has the following amino acid sequence:

(SEQ ID NO: 7)

MRIFAGIIFTACCHLLRAFTITAPKDLYVVEYGSNVTMECRFPVERELDL
LALVVYWEKEDEQVIQFVAGEEDLKPQHSNFRGRASLPKDQLLKGNAALQ
ITDVKLQDAGVYCCIISYGGADYKRITLKVNAPYRKINQRISVDPATSEH
ELICQAEGYPEAEVIWTNSDHQPVSGKRSVTTSRTEGMLLNVTSSLRVNA
TANDVFYCTFWRSQPGQNHTAELIIPELPATHPPQNRTHWVLLGSILLFL
IVVSTVLLFLRKQVRMLDVEKCGVEDTSSKNRNDTQFEET.

In some instances, the provided methods may include measuring the level and/or detecting overexpression of the immune checkpoint protein CD47. In humans, CD47 (also known as integrin-associated protein (IAP), OA3 and MERG) is encoded by the CD47 gene (NCBI Gene ID: 961) present on chromosome 3 at 3q13.12. CD47 is a ubiquitously expressed membrane glycoprotein of the immunoglobulin superfamily that plays a critical role in self-recognition. Various solid and hematologic cancers exploit CD47 expression in order to evade immunological eradication, and its overexpression is clinically correlated with poor prognoses. One essential mechanism behind CD47-mediated immune evasion is that it can interact with signal regulatory protein-alpha (SIRPα) expressed on myeloid cells.

Human CD47 protein (GenBank Accession No. NP_001768.1, NP_942088.1; UniProt Q08722) has the following four different isoform amino acid sequences: Isoform OA3-323:

(SEQ ID NO: 8)

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLNAFKESKGMMNDE;

### Isoform OA3-293:

(SEQ ID NO: 9)

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFV;

### Isoform OA3-305:

(SEQ ID NO: 10)

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRNN;

### Isoform OA3-312:

(SEQ ID NO: 11)

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLN.

In mice, CD47 is encoded by the CD47 gene (also known as IAP, Itgp, AA407862, A1848868, AW108519, 9130415E20Rik and B430305P08Rik; NCBI Gene ID: 16423) present on chromosome 16 at 16 B5. Mouse CD47 protein (GenBank Accession No. NP_034711.1; UniProt Q61735) has the following amino acid sequence:

(SEQ ID NO: 12)

MWPLAAALLLGSCCCGSAQLLFSNVNSIEFTSCNETVVIPCIVRNVEAQS
TEEMFVKWKLNKSYIFIYDGNKNSTTTDQNFTSAKISVSDLINGIASLKM
DKRDAMVGNYTCEVTELSREGKTVIELKNRTVSWFSPNEKILIVIFPILA
ILLFWGKFGILTLKYKSSHTNKRIILLLVAGLVLTVIVVVGAILLIPGEK
PVKNASGLGLIVISTGILILLQYNVFMTAFGMTSFTIAILITQVLGYVLA
LVGLCLCIMACEPVHGPLLISGLGIIALAELLGLVYMKFVASNQRTIQPP
RNR.

In some instances, the above provided proteins, the amino acid sequences thereof (e.g., related to MYC, PD-L1, CD47, etc.) and/or nucleic acids encoding the proteins or amino acid sequences may be targeted with one or more therapeutic agents, as described in more detail below.

Therapeutic Agents

In some instances, the methods of the present disclosure include treating a subject for a neoplasm by administering the subject an effective amount of one or more therapeutic agents. Therapeutic agents useful in the subject methods of treatment include inhibitors, i.e., agents that inhibit the production, function or activity of a target molecule. For example, useful inhibitors include “immune checkpoint inhibitors” that inhibit the production, function or activity of an immune checkpoint molecule and “MYC inhibitors” that inhibit the production, function or activity of MYC.

Any useful inhibitor of a subject target gene (e.g., an immune checkpoint gene or a MYC gene) and/or an encoded product thereof (e.g., an immune checkpoint protein or a MYC protein) may be employed in the subject methods. Non-limiting examples of useful inhibitors include but are not limited to e.g., non-peptide small molecule antagonists, peptide antagonists, interfering RNAs (e.g., siRNA, shRNA, etc.), antibodies (e.g., neutralizing antibodies, function blocking antibodies, etc.), aptamers, and the like. Accordingly, as non-limiting examples, in some instances, useful inhibitors may include a non-peptide small molecule antagonist of an immune checkpoint protein, a non-peptide small molecule antagonist of MYC, a peptide antagonist of an immune checkpoint protein, a peptide antagonist of MYC, an interfering RNA targeting an RNA expressed from an immune checkpoint protein encoding gene, an interfering RNA targeting an RNA expressed from a MYC encoding gene, an anti-immune checkpoint protein antibody (e.g., an antibody that specifically binds to an immune checkpoint protein), an anti-MYC antibody (e.g., an antibody that specifically binds to a MYC protein), an anti-immune checkpoint protein aptamer, an anti-MYC aptamers, and the like. In some instances, the effectiveness of an inhibitor may be confirmed using an in vitro or in vivo assay, including e.g., where the effectiveness of the inhibitor is compared to an appropriate control or standard, e.g., a conventional immune checkpoint inhibitor, a conventional MYC inhibitor, a conventional therapy for the condition, etc.

An inhibitory agent may specifically bind to the target molecule (e.g., protein or mRNA) that it inhibits. For example, a MYC inhibitor may specifically bind MYC protein or a nucleic acid encoding MYC, a PD-L1 inhibitor may specifically bind PD-L1 protein or a nucleic acid encoding PD-L1, a CD47 inhibitor may specifically bind CD47 protein or a nucleic acid encoding CD47.

In some instances, the inhibitor (e.g., an antibody) may specifically bind to a protein having 100% or less amino acid sequence identity with a MYC amino acid sequence (including e.g., SEQ ID NOs:1-3), including e.g., where the protein has 80% or greater amino acid sequence identity with the MYC amino acid sequence, including 85% or greater, 90% or greater, 95% or greater, 96% or greater, 97% or greater, 98% or greater, 99% or greater, etc. In some instances, the inhibitor (e.g., an interfering RNA) may specifically bind to a nucleic acid encoding MYC or a protein having 100% or less amino acid sequence identity with a MYC amino acid sequence (including e.g., SEQ ID NOs:1-3), including e.g., where the protein has 80% or greater amino acid sequence identity with the MYC amino acid sequence, including 85% or greater, 90% or greater, 95% or greater, 96% or greater, 97% or greater, 98% or greater, 99% or greater, etc.

In some instances, the inhibitor (e.g., an antibody) may specifically bind to a protein having 100% or less amino acid sequence identity with a PD-L1 amino acid sequence (including e.g., SEQ ID NOs:4-7), including e.g., where the protein has 80% or greater amino acid sequence identity with the PD-L1 amino acid sequence, including 85% or greater, 90% or greater, 95% or greater, 96% or greater, 97% or greater, 98% or greater, 99% or greater, etc. In some instances, the inhibitor (e.g., an interfering RNA) may specifically bind to a nucleic acid encoding PD-L1 or a protein having 100% or less amino acid sequence identity with a PD-L1 amino acid sequence (including e.g., SEQ ID NOs:4-7), including e.g., where the protein has 80% or greater amino acid sequence identity with the PD-L1 amino acid sequence, including 85% or greater, 90% or greater, 95% or greater, 96% or greater, 97% or greater, 98% or greater, 99% or greater, etc.

In some instances, the provided methods may include administering an inhibitor of a PD-L1 receptor, e.g., PD1. In humans, PD1 (programmed cell death protein 1, also known as CD279 (cluster of differentiation 279), SLEB2, hPD-1, hPD-I and hSLE1) is a cell surface receptor that plays an important role in down-regulating the immune system and promoting self-tolerance by suppressing T cell inflammatory activity. PD1 is an immune checkpoint and guards against autoimmunity through a dual mechanism of promoting apoptosis (programmed cell death) in antigen specific T-cells in lymph nodes while simultaneously reducing apoptosis in regulatory T cells (anti-inflammatory, suppressive T cells).

PD1 is encoded by the PDCD1 gene (NCBI Gene ID: 5133) in humans, present on chromosome 2 at 2q37.3. Human PD1 protein (GenBank Accession No. NP_005009.2; UniProt Q15116) has the following amino acid sequence:

(SEQ ID NO: 13)

MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

In some instances, the inhibitor (e.g., an antibody) may specifically bind to a protein having 100% or less amino acid sequence identity with a PD1 amino acid sequence (including e.g., SEQ ID NO:13), including e.g., where the protein has 80% or greater amino acid sequence identity with the PD1 amino acid sequence, including 85% or greater, 90% or greater, 95% or greater, 96% or greater, 97% or greater, 98% or greater, 99% or greater, etc. In some instances, the inhibitor (e.g., an interfering RNA) may specifically bind to a nucleic acid encoding PD1 or a protein having 100% or less amino acid sequence identity with a PD1 amino acid sequence (including e.g., SEQ ID NO:13), including e.g., where the protein has 80% or greater amino acid sequence identity with the PD1 amino acid sequence, including 85% or greater, 90% or greater, 95% or greater, 96% or greater, 97% or greater, 98% or greater, 99% or greater, etc.

In some instances, the inhibitor (e.g., an antibody) may specifically bind to a protein having 100% or less amino acid sequence identity with a CD47 amino acid sequence (including e.g., SEQ ID NOs:8-12), including e.g., where the protein has 80% or greater amino acid sequence identity with the CD47 amino acid sequence, including 85% or greater, 90% or greater, 95% or greater, 96% or greater, 97% or greater, 98% or greater, 99% or greater, etc. In some instances, the inhibitor (e.g., an interfering RNA) may specifically bind to a nucleic acid encoding CD47 or a protein having 100% or less amino acid sequence identity with a CD47 amino acid sequence (including e.g., SEQ ID NOs:8-12), including e.g., where the protein has 80% or greater amino acid sequence identity with the CD47 amino acid sequence, including 85% or greater, 90% or greater, 95% or greater, 96% or greater, 97% or greater, 98% or greater, 99% or greater, etc.

In some instances, the provided methods may include administering an inhibitor of a CD47 receptor, e.g., SIRPα. In humans, SIRPα (Signal regulatory protein α, also known as Tyrosine-protein phosphatase non-receptor type substrate 1,) is an immunoglobulin-like cell surface receptor regulatory membrane glycoprotein from SIRP family that acts as inhibitory receptor and interacts with CD47, also called the “don't eat me” signal. This interaction negatively controls effector function of innate immune cells such as host cell phagocytosis, analogous to the self-signals provided by MHC class I molecules to NK cells via Ig-like or Ly49 receptors.

SIRPα is encoded by the signal regulatory protein alpha gene (NCBI Gene ID: 140885), in humans present on chromosome 20 at 20p13. Human SIRPα protein (GenBank Accession No. NP_001035111.1, NP_001035112.1, NP_001317657.1, NP_542970.1; UniProt P78324) has the following 3 different isoform amino acid sequences:

### Isoform 1:

(SEQ ID NO: 14)

MEPAGPAPGRLGPLLCLLLAASCAWSGVAGEEELQVIQPDKSVLVAAGET
ATLRCTATSLIPVGPIQWFRGAGPGRELIYNQKEGHFPRVTTVSDLTKRN
NMDFSIRIGNITPADAGTYYCVKFRKGSPDDVEFKSGAGTELSVRAKPSA
PVVSGPAARATPQHTVSFTCESHGFSPRDITLKWFKNGNELSDFQTNVDP
VGESVSYSIHSTAKVVLTREDVHSQVICEVAHVTLQGDPLRGTANLSETI
RVPPTLEVTQQPVRAENQVNVTCQVRKFYPQRLQLTWLENGNVSRTETAS
TVTENKDGTYNWMSWLLVNVSAHRDDVKLTCQVEHDGQPAVSKSHDLKVS
AHPKEQGSNTAAENTGSNERNIYIVVGVVCTLLVALLMAALYLVRIRQKK
AQGSTSSTRLHEPEKNAREITQDTNDITYADLNLPKGKKPAPQAAEPNNH
TEYASIQTSPQPASEDTLTYADLDMVHLNRTPKQPAPKPEPSFSEYASVQ
VPRK;

### Isoform 2:

(SEQ ID NO: 15)

MEPAGPAPGRLGPLLCLLLAASCAWSGVAGEEELQVIQPDKSVLVAAGET
ATLRCTATSLIPVGPIQWFRGAGPGRELIYNQKEGHFPRVTTVSDLTKRN
NMDFSIRIGNITPADAGTYYCVKFRKGSPDDVEFKSGAGTELSVRAKPSA
PVVSGPAARATPQHTVSFTCESHGFSPRDITLKWFKNGNELSDFQTNVDP
VGESVSYSIHSTAKVVLTREDVHSQVICEVAHVTLQGDPLRGTANLSETI
RVPPTLEVTQQPVRAENQVNVTCQVRKFYPQRLQLTWLENGNVSRTETAS
TVTENKDGTYNWMSWLLVNVSAHRDDVKLTCQVEHDGQPAVSKSHDLKVS
AHPKEQGSNTAAENTGSNERNIYIVVGVVCTLLVALLMAALYLVRIRQKK
AQGSTSSTRLHEPEKNAREITQVQSLDTNDITYADLNLPKGKKPAPQAAE
PNNHTEYASIQTSPQPASEDTLTYADLDMVHLNRTPKQPAPKPEPSFSEY
ASVQVPRK;

### Isoform 3:

(SEQ ID NO: 16)

MEPAGPAPGRLGPLLCLLLAASCAWSGVAGEEELQVIQPDKSVLVAAGET
ATLRCTATSLIPVGPIQWFRGAGPGRELIYNQKEGHFPRVTTVSDLTKRN
NMDFSIRIGNITPADAGTYYCVKFRKGSPDVEFKSGAGTELSVRAKPSAP
VVSGPAARATPQHTVSFTCESHGFSPRDITLKWFKNGNELSDFQTNVDPV
GESVSYSIHSTAKVVLTREDVHSQVICEVAHVTLQGDPLRGTANLSETIR
VPPTLEVTQQPVRAENQVNVTCQVRKFYPQRLQLTWLENGNVSRTETAST
VTENKDGTYNWMSWLLVNVSAHRDDVKLTCQVEHDGQPAVSKSHDLKVSA
HPKEQGSNTAAENTGSNERNIYIVVGVVCTLLVALLMAALYLVRIRQKKA
QGSTSSTRLHEPEKNAREITQDTNDITYADLNLPKGKKPAPQAAEPNNHT
EYASIQTSPQPASEDTLTYADLDMVHLNRTPKQPAPKPEPSFSEYASVQV
PRK.

In some instances, the inhibitor (e.g., an antibody) may specifically bind to a protein having 100% or less amino acid sequence identity with a SIRPα amino acid sequence (including e.g., SEQ ID NOs:14-16), including e.g., where the protein has 80% or greater amino acid sequence identity with the SIRPα amino acid sequence, including 85% or greater, 90% or greater, 95% or greater, 96% or greater, 97% or greater, 98% or greater, 99% or greater, etc. In some instances, the inhibitor (e.g., an interfering RNA) may specifically bind to a nucleic acid encoding SIRPα or a protein having 100% or less amino acid sequence identity with a SIRPα amino acid sequence (including e.g., SEQ ID NOs:14-16), including e.g., where the protein has 80% or greater amino acid sequence identity with the SIRPα amino acid sequence, including 85% or greater, 90% or greater, 95% or greater, 96% or greater, 97% or greater, 98% or greater, 99% or greater, etc.

As summarized above, the provided methods may, in some embodiments, involve the administration of one or more MYC inhibitors, including e.g., two or more MYC inhibitors, e.g., 3 or more, 4 or more, 5 or more, etc., to a subject. Any MYC inhibitor, including inhibitors that directly target MYC and those that indirectly inhibit MYC signaling may be employed.

Useful MYC inhibitors include but are not limited to small molecule direct and indirect inhibitors of MYC. Non-limiting examples of small molecule direct and indirect inhibitors of MYC include: IIA6B17 (Berg et al., 2002) and analogs thereof (e.g., mycmycin-1;), IIA4B20 (Berg et al., 2002) and analogs thereof (e.g., mycmycin-2); 2-[3-oxo-3-(1-piperidinyl)propyl]-1H-isoindole-1,3(2H)-dione (MW 286.3) and analogs thereof; 5-(4-chlorobenzylidene)-2-thioxo-1,3-thiazolidin-4-one (MW 255.7) and analogs thereof; 2-(4-ethylphenoxy)-N-(2-phenylethyl)acetamide (MW 283.4) and analogs thereof; 4-methyl-2-[2-(6-methyl-2-phenyl-2,3-dihydro-4H-chromen-4-ylidene)hydrazino]-N-(3-nitrophenyl)-1,3-thiazole-5-carboxamide hydrobromide (MW 594.5) and analogs thereof; 5-(3,4-dihydroxybenzylidene)-2-thioxo-1,3-thiazolidin-4-one (MW 253.3) and analogs thereof; 5-(4-isopropylbenzylidene)-2-thioxo-1,3-thiazolidin-4-one (MW 263.4) and analogs thereof; 3-[3-(3,6-dichloro-9H-carbazol-9-yl)-2-hydroxypropyl]-1,3-thiazolidine-2,4-dione (MW 409.3) and analogs thereof; N-2-biphenylyl-7-nitro-2,1,3-benzoxadiazol-4-amine (MW 332.3) and analogs thereof; 3-[2-(4-ethylphenyl)-2-oxoethyl]-6,7-dimethoxy-2-benzofuran-1(3H)-one (MW 340.4) and analogs thereof; 1-(3-chlorophenyl)-3-(diethylamino)-2,5-pyrrolidinedione (MW 280.8) and analogs thereof; N-(2-bromo-4-methylphenyl)-2-(4-ethylphenoxy)acetamide (MW 348.2) and analogs thereof; 5-(4-ethylbenzylidene)-2-thioxo-1,3-thiazolidin-4-one (MW 249.4) and analogs thereof; 6-bromo-3-[3-(3-nitrophenyl)acryloyl]-4-phenyl-2(1H)-quinolinone (MW 475.3) and analogs thereof; 1-[2,5-dioxo-1-(4-propoxyphenyl)-3-pyrrolidinyl]-4-piperidinecarboxylic acid (MW 360.4) and analogs thereof; N-[2-(4-nitrophenyl)ethyl]bicyclo[2.2.1]heptan-2-amine (MW 260.3) and analogs thereof; N-(4-methoxybenzyl)-5-(2-thienyl)-7-(trifluoromethyl)pyrazolo[1,5-a]pyrimidine-2-carboxamide (MW 432.4) and analogs thereof; 2-(4-ethylphenoxy)-N-(1-naphthylmethyl)acetamide (MW 319.4) and analogs thereof; butyl 3-[5-(4-ethylbenzylidene)-4-oxo-2-thioxo-1,3-thiazolidin-3-yl]propanoate (MW 377.5) and analogs thereof; 1-[2-(2,5-dimethylphenoxy)ethyl]-1H-indole-3-carbaldehyde (MW 293.4) and analogs thereof; N-1,3-benzothiazol-2-yl-2-(4-isopropylphenoxy)acetamide (MW 326.4) and analogs thereof; methyl 1-(4-ethylphenyl)-5-oxo-3-pyrrolidinecarboxylate (MW 247.3) and analogs thereof; 5-(4-ethylbenzylidene)-3-(2-propyn-1-yl)-1,3-thiazolidine-2,4-dione (MW 271.3) and analogs thereof; 3-allyl-5-(4-ethylbenzylidene)-2-thioxo-1,3-thiazolidin-4-one (MW 289.4) and analogs thereof; N-[2-(4-methoxyphenyl)ethyl]-4-(methylthio)benzenesulfonamide (MW 337.5) and analogs thereof; NZY2267 (Xu et al., 2006) and analogs thereof; Mycro1 (Kiessling et al., 2006) and analogs thereof; Mycro2 (Kiessling et al., 2006) and analogs thereof; “Compounds 1-5” as described in Kiessling et al., Chem Med Chem. 2007; 2(5):627-30 and analogs thereof; 10058-F4 (Yin et al., 2003) and analogs thereof; 10074-G5 (Yin et al., 2003) and analogs thereof; JY-3-094 (Yap et al., 2013) and analogs thereof; SF-4-017 (Yap et al., 2013) and analogs thereof; bromodomain and extra-terminal (BET) domain inhibitors (e.g., JQ1 and analogs thereof; I-BET762 and analogs thereof; I-BET151 and analogs thereof; MS417 and analogs thereof; and MS436 and analogs thereof; and the like); PKUMDL-YC-1101 (Yu et al. 2016) an analogs thereof, PKUMDL-YC-1201 (Yu et al. 2016) an analogs thereof, PKUMDL-YC-1202 (Yu et al. 2016) an analogs thereof, PKUMDL-YC-1203 (Yu et al. 2016) an analogs thereof, PKUMDL-YC-1204 (Yu et al. 2016) an analogs thereof, and PKUMDL-YC-1301 (Yu et al. 2016) an analogs thereof; PKUMDL-YC-1205 (Yu et al. 2016) an analogs thereof;

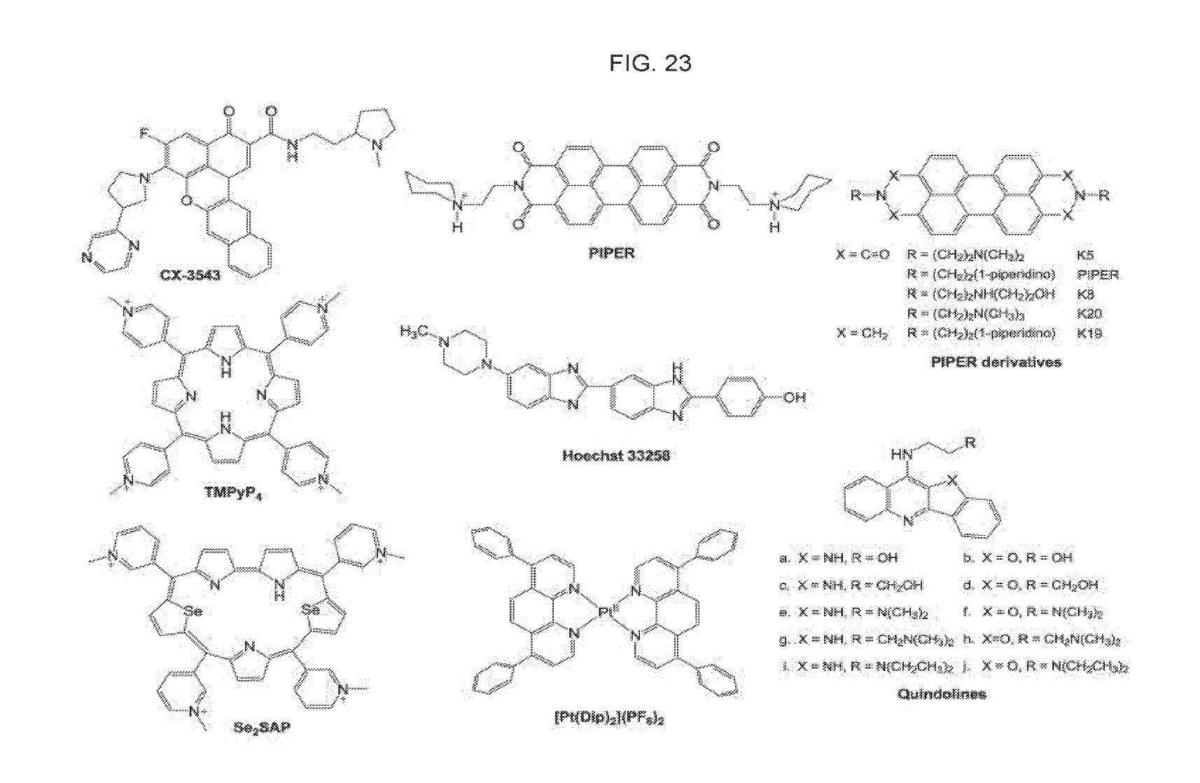

Useful MYC inhibitors may also include, in some instances, small molecules that bind c-Myc promoter G-quadruplexes, including but not limited to e.g., CX-3543 (Quarfloxin; see e.g., Chen et al., Int J Biol Sci. 2014; 10(10): 1084-1096). C-Myc promoter G-quadruplexe binding MYC inhibitors include but are not limited to e.g., those molecules depicted in FIG. 23 and analogs thereof.

Useful MYC inhibitors may also include, in some instances, small molecules that prevent or interfere with the association of MYC with its binding partner MAX (i.e., molecules that prevent MYC/MAX binding). Such molecules may directly/specifically bind MYC (e.g., at a MAX binding site) or may directly/specifically bind MAX (e.g., at a MYC binding site). Non-limiting examples include but are not limited to e.g., those described in U.S. Pat. No. 8,637,556; the disclosure of which is incorporated herein by reference in its entirety.

Useful MYC inhibitors may also include peptide inhibitors of MYC. Non-limiting examples of peptide inhibitors of MYC include Myc H1 DNA-binding region based peptide MYC inhibitors (including e.g., peptides having the H1 DNA-binding region of c-Myc containing Ser to Ala, and Phe to Ala substitutions and cell permeable versions thereof; see e.g., Giorello, et al.; Cancer Res. 58, 3654 (1998)). Examples of Helix 1 (H1) MYC inhibitory peptides include Bac-ELP1-H1 and Bac-ELP2-H1, such peptides have the following amino acid sequence:

(SEQ ID NO: 17)

MRRIRPRPPRLPRPRPRPLPFPRPGGCYPG-(VPGXG)ₙ-WPGSGNELK
RAFAALRDQI,

where ELP1-H1 contains V, G, or A at the X position in a 5:3:2 ratio, respectively, and n is 150 and Bac-ELP2-H1 contains V, G, or A at the X position in a 1:7:8 ratio, respectively, and n is 160 (see e.g., Bidwell, et al., PLoS One. 2013; 8(1):e55104). MYC inhibitory peptides generally include any peptide that prevents MYC binding of endogenous DNA targets preventing MYC transcription factor activity.

Other examples of peptide inhibitors of MYC include but are not limited to e.g., basic-helix-loop-helix-leucine zipper (bHLHZ) motif containing peptides. Such peptides may include a MYC bHLHZ domain or a MAX bHLHZ domain but exclude one or more MYC or MAX functional domains, including e.g., where such a peptide includes a MYC bHLHZ domain but excludes a MYC transactivation domain (TAD). MYC or MAX bHLHZ domains may be mutated at one or more amino acid residues, including e.g., 1, 2, 3, 4, 5, 6, 7, 8, or more residues. Peptide inhibitors of MYC containing a bHLHZ motif may function as a dominate negative MYC. In some instances, a bHLHZ motif containing peptide inhibitor of MYC may include the following mutated MYC bHLHZ domain amino acid sequence:

(SEQ ID NO: 18)

QAETQKLISEIDLLRKQNEQLKHKLEQL,

or have 100% or less amino acid sequence identity, including 99% or greater sequence identity, 95% or greater sequence identity, 90% or greater sequence identity, 85% or greater sequence identity, etc., with the mutated MYC bHLHZ domain amino acid sequence of SEQ ID NO:18). One non-limiting example of a bHLHZ motif containing peptide inhibitors of MYC is OmoMYC, which has the following amino acid sequence:

(SEQ ID NO: 19)

TEENVKRRTHNVLERQRRNELKRSFFALRDQIPELENNEKAPKVVILKKA
TAYILSVQAETQKLISEIDLLRKQNEQLKHKLEQLRNSCA.

In some instances, useful peptide inhibitors of MYC will include analogs or mutants of OmoMYC including but not limited to e.g., those having one or more mutated amino acid residues as compared to SEQ ID NO:19, including e.g., 1, 2, 3, 4, 5, 6, 7, 8, or more mutated residues and those having less than 100% sequence identity with SEQ ID NO:19, including 99% or greater sequence identity, 95% or greater sequence identity, 90% or greater sequence identity, 85% or greater sequence identity, etc., and the like. Useful bHLHZ MYC/MAX inhibitors include but are not limited to e.g., those describe in US Patent Application Pub. No. US 2016/0122415 A1, Soucek et al., Oncogene. 1998; 17(19):2463-72; Soucek et al., Cancer Res. 2002; 62(12):3507-10; Savino et al., PLoS One. 2011; 6(7):e22284; the disclosures of which are incorporated herein by reference in their entirety.

Useful MYC inhibitors may also include MYC inhibitory nucleic acids. By “MYC inhibitory nucleic acids” is generally meant nucleic acid molecules that prevent the expression and/or translation of MYC protein. MYC inhibitory nucleic acids include MYC interfering RNAs of about 5 to 50 bp or 10 to 30 bp, which are capable of binding to c-Myc gene or transcript thereof including e.g., MYC-directed siRNA, MYC-directed shRNA and the like. Non-limiting examples of MYC interfering RNAs include e.g., those anti-MYC shRNAs and siRNAs directed to MYC encoding nucleic acids, including e.g., c-Myc siRNA, shRNA and Lentiviral Particle Gene Silencers (available from Santa Cruz Biotechnology, Inc.; Santa Cruz, Calif.)). Non-limiting examples of c-Myc siRNA include but are not limited to e.g., those described in U.S. Pat. No. 9,457,043; the disclosure of which is incorporated herein by reference in its entirety.

MYC inhibitors include but are not limited to those described in Berg et al., Proc Natl Acad Sci USA. 2002; 99(6):3830-5; Wang et al., Oncogene. 2008; 27:1905-1915; Yin et al., Oncogene. 2003; 22(40):6151-9; Shi et al., Bioorg. Med. Chem. Lett. 2009; 19:6038-6041; Xu et al., Bioorg Med Chem. 2006 Apr. 15; 14(8):2660-73; Kiessling et al., Chem Biol. 2006; 13(7):745-51; Kiessling et al., Chem Med Chem. 2007; 2(5):627-30; Yap et al., Bioorg Med Chem Lett. 2013; 23(1):370-4; Segura et al., Cancer Res. 2013; 73(20):6264-76; Fletcher & Prochownik, Biochim Biophys Acta. 2015; 1849(5): 525-543; Yu et al., Sci Rep. 2016 Mar. 2; 6:22298; Chen et al., Int J Biol Sci. 2014; 10(10): 1084-1096; Bidwell, et al., PLoS One. 2013; 8(1):e55104; Prochownik & Vogt, Genes Cancer. 2010 June; 1(6): 650-659; U.S. Pat. Nos. 9,567,301; 9,457,043; 9,387,213; 9,506,060; 9,278,940; 9,228,189 and 9,155,724; the disclosures of which are incorporated herein by reference in their entirety.

MYC inhibitors may be administered to a subject in an effective amount, where an effective amount of a MYC inhibitor may be an amount effective to achieve the desired response and/or treat the individual. Effective amounts of MYC inhibitors may be determined empirically, e.g., through one or more clinical trials, inferred from one or more pharmacological studies and/or preclinical animal studies, or other means.

In certain embodiments, an effective amount of a MYC inhibitor for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a MYC inhibitor may be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

Any other anti-MYC therapy may be employed in the subject methods where inhibition of MYC and/or targeting of MYC overexpressing cells is desired, including but not limited to e.g., targeting c-MYC using T cells as described in e.g., Helm et al., PLoS ONE 8(10): e77375; the disclosure of which is incorporated by reference herein in its entirety.

As summarized above, the provided methods may include administering to a subject one or more immune checkpoint inhibitors, including but not limited to e.g., where the subject is administered two or more immune checkpoint inhibitors, e.g., 3 or more, 4 or more, 5 or more, etc. Useful immune checkpoint inhibitors include inhibitors of one or more immune checkpoint proteins, including e.g., agents that specifically bind an immune checkpoint ligand or an immune checkpoint receptor or otherwise prevents the association of an immune checkpoint ligand with its receptor. Non-limiting examples of immune checkpoint inhibitors include drugs that target PD1, drugs that target PD-L1, drugs that target CLTA4 (e.g., Ipilimumab (Yervoy)), drugs that target CD47, drugs that target Signal Regulatory Protein α (SIRPα, aka CD172a or SHPS-1), and the like.

The provided methods may, in some embodiments, involve the administration of one or more PD1/PD-L1 pathway inhibitors, including e.g., two or more PD1/PD-L1 pathway inhibitors, e.g., 3 or more, 4 or more, 5 or more, etc., to a subject. Any PD1/PD-L1 pathway inhibitor, including inhibitors that directly target PD1 or PD-L1 and those that indirectly inhibit PD1/PD-L1 pathway signaling may be employed. As such, useful inhibitors of the PD1/PD-L1 pathway include agents (e.g., small molecules, antibodies, inhibitory nucleic acids, etc.) that specifically bind PD-L1, specifically bind PD1 and/or prevent the association of PD-L1 with PD1, inhibit the expression PD-L1, inhibit the expression of PD1, and the like.

Useful PD-L1/PD1 pathway inhibitors include antibodies that specifically bind to PD-L1 or PD1, inhibit the PD-L1/PD1 interaction and/or otherwise prevent PD-L1/PD1 pathway signaling. Non-limiting examples of such antibodies include: Durvalumab (aka, anti-B7H1 monoclonal antibody, MEDI-4736; AstraZeneca/Medlmmune); avelumab (aka, MSB-0010718C; a fully human monoclonal PD-L1 antibody of isotype IgG1; Merck); Atezolizumab (aka, MPDL3280A, Tecentriq); Nivolumab (aka Opdivo, BMS-936558 or MDX1106b; a human IgG4 antibody against PD-1, lacking detectable antibody-dependent cellular toxicity (ADCC); Bristol-Myers Squibb); Pembrolizumab (aka MK-3475, lambrolizumab, KEYTRUDA; an highly specific anti-PD-1 humanized IgG4 isotype antibody that contains a mutation at C228P designed to prevent Fc-mediated cytotoxicity); Pidilizumab (aka CT-011, MDV9300; a humanized monoclonal antibody (mAb) that modulates the immune response and inhibits tumor growth and the spread of metastases; CureTech Ltd.); and the like.

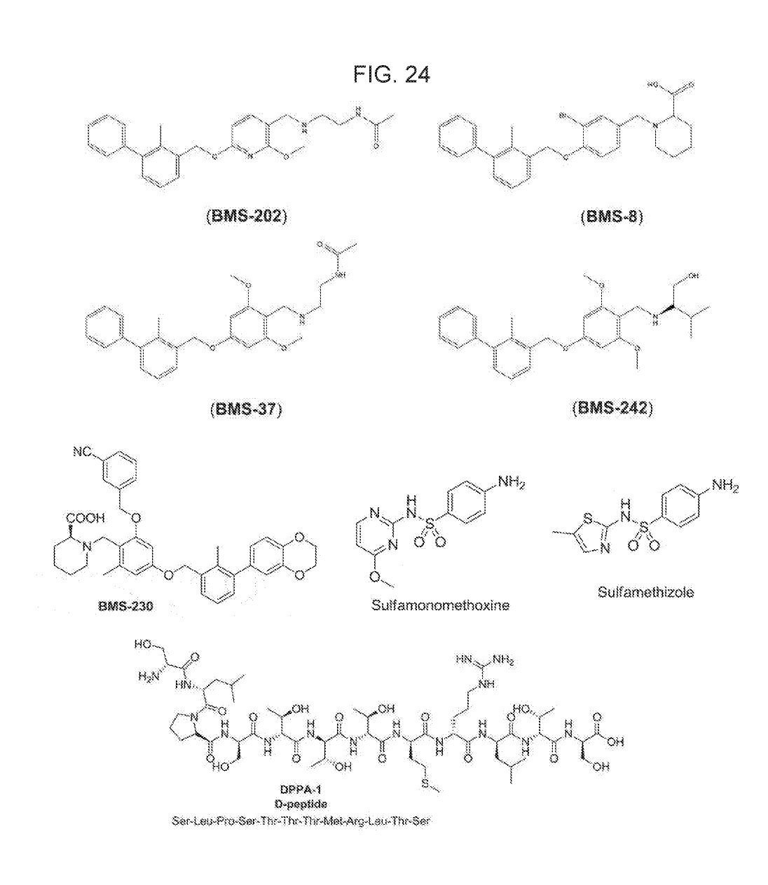

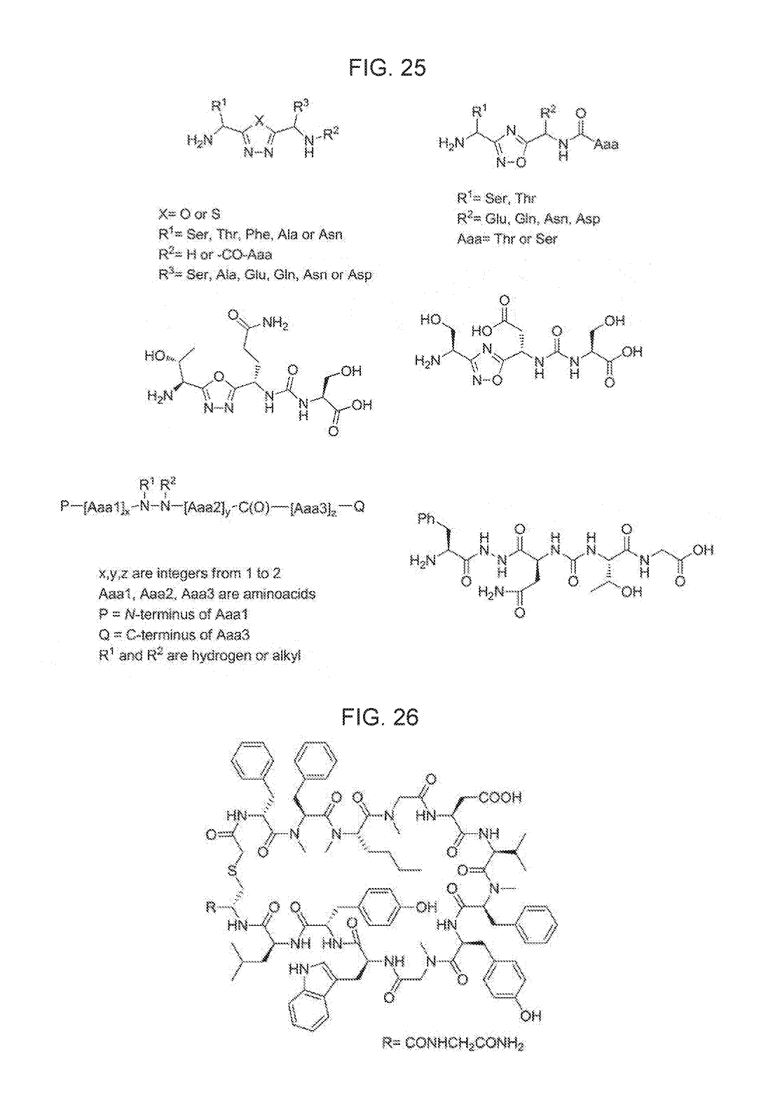

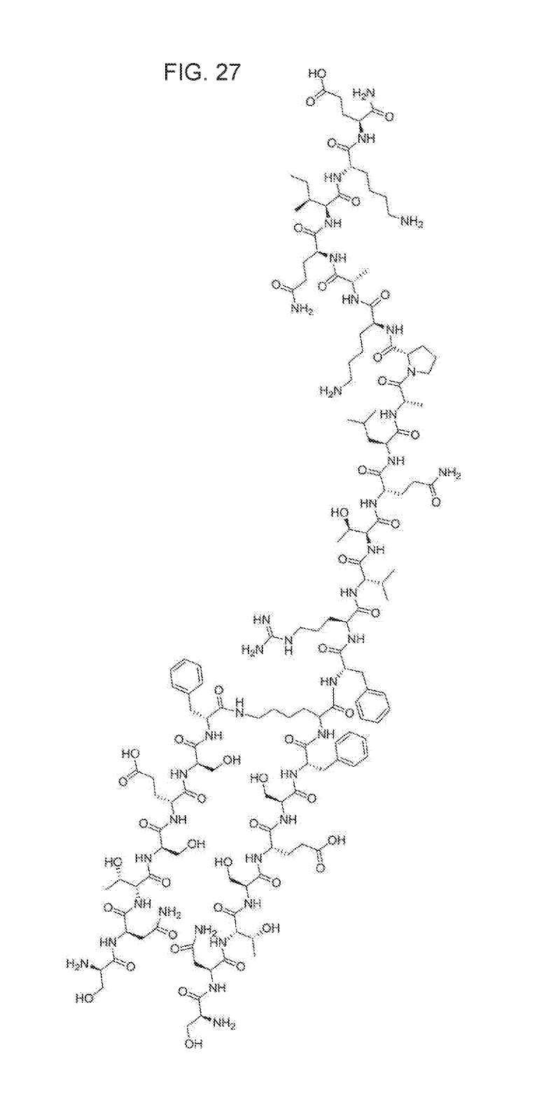

Useful PD-L1/PD1 pathway inhibitors include small molecules (e.g., peptidomimetics, macrocyclic peptides, nonpeptidic molecules, and the like) that specifically bind to PD-L1 or PD1, inhibit the PD-L1/PD1 interaction and/or otherwise prevent PD-L1/PD1 pathway signaling. Non-limiting examples of small molecule PD-L1/PD1 pathway inhibitors include 2-methyl-3-biphenylyl)methanol based derivatives (e.g., BMS-202, BMS-8 (FIG. 24) BMS-37, and BMS-242 as depicted in FIG. 24 and analogs thereof); 1,2,4-oxadiazole- and 1,2,4-thiadiazole-based inhibitors and analogs thereof; CA-327 (Curis, Inc., Lexington, Mass.); CA-327 (Curis, Inc., Lexington, Mass.); sulphonamide derivatives (e.g., sulfamonomethoxine and sulfamethizole as depicted in FIG. 24 and analogs thereof); peptidomimetic inhibitors of the PD-1/PD-L1 interaction (e.g., those having oxa- and thiadiazole core moieties as depicted in FIG. 25); D-peptide PD1/PD-L1 antagonists (e.g., DPPA-1 as depicted in FIG. 24 and analogs thereof); Macrocyclic peptidic PD1/PD-L1 inhibitors (e.g., as depicted in FIG. 26 and analogs thereof); peptide antagonists of PD-L1 or PD1 (such as e.g., the peptide antagonists of PD-L1 depicted in FIG. 27).

PD-L1/PD1 inhibitors include but are not limited to those described in Zak et al., Oncotarget. 2016; 7(21):30323-35; Zhan et al., Drug Discov Today. 2016 June; 21(6):1027-36; Chang et al., Angew Chem Int Ed. 2015; 54:11760-11764; Zarganes-Tzitzikas et al., Expert Opin Ther Pat. 2016 September; 26(9):973-7; U.S. Pat. Nos. 6,808,710; 7,029,674; 7,101,550; 7,488,802; 7,722,868; 8,008,449; 8,088,905; 8,168,757; 8,383,796; 8,709,416; 8,741,295; 8,951,518; 8,952,136; 8,993,731; 9,102,728; 9,163,087; 9,175,082; 9,181,342; 9,205,148; 9,212,224; 9,217,034; 9,220,776; 9,273,135; 9,492,539; US Patent Application Pub. Nos. 20110318373; 20130095098; 20130133091; 20130323249; 20140227262; 20140234296; 20140335093; 20140348743; 20140356363; 20150073024; 20150118234; 20150152180; 20150203579; 20150203580; 20150210769; 20150315274; 20150346208; 20150355184; 20160031990; 20160068586; 20160075782; 20160075783; 20160130348; 20160137731; 20160222113; 20160304606; 20160333096; 20160362495; 20160376367; 20170007693; 20170044259; 20170044260; PCT Pub. Nos. WO/2015/034820 and WO/2011/082400; the disclosures of which are incorporated herein by reference in their entirety.

PD-L1/PD1 inhibitors may be administered to a subject in an effective amount, where an effective amount of a PD-L1/PD1 inhibitor may be an amount effective to achieve the desired response and/or treat the individual. Effective amounts of PD-L1/PD1 inhibitors may be determined empirically, e.g., through one or more clinical trials, inferred from one or more pharmacological studies and/or preclinical animal studies, or other means.

In certain embodiments, an effective amount of a PD-L1/PD1inhibitor for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a PD-L1/PD1inhibitor may be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

The provided methods may, in some embodiments, involve the administration of one or more CD47/SIRPα pathway inhibitors, including e.g., two or more CD47/SIRPα pathway inhibitors, e.g., 3 or more, 4 or more, 5 or more, etc., to a subject. Any CD47/SIRPα pathway inhibitor, including inhibitors that directly target CD47 or SIRPα, inhibit the interaction of CD47 and SIRPα and/or those that indirectly inhibit CD47/SIRPα pathway signaling may be employed. As such, useful inhibitors of the CD47/SIRPα pathway include agents (e.g., small molecules, antibodies, inhibitory nucleic acids, etc.) that specifically bind CD47, specifically bind SIRPα and/or prevent the association of CD47 with SIRPα, inhibit the expression CD47, inhibit the expression of SIRPα, and the like.

Useful CD47/SIRPα pathway inhibitors include but are not limited to antibodies, including e.g., antibodies that specifically bind CD47, antibodies that specifically bind SIRPα, and the like. Non-limiting examples of such antibodies include e.g., CC-900002 (an anti-CD47 antibody developed by Celgene Corporation, Summit, N.J.), Hu5F9-G4 (humanized monoclonal antibody against CD47; Forty Seven, Inc., Menlo Park, Calif.), anti-CD47 antibodies developed by Tioma Therapeutics (Brisbane, Calif.), as well as other anti-CD47/SIRPα antibodies developed by these and other companies.

Useful CD47/SIRPα pathway inhibitors include inhibitory peptides. Inhibitory peptides, as generally referred to herein with regards to CD47/SIRPα and other listed target molecules (e.g., PD1, PD-L1, MYC, etc.), include peptides that are fused to a heterologous peptide/polypeptide (i.e., fusion proteins) as well as peptides that are not fused a heterologous peptide/polypeptide. For example, useful CD47/SIRPα peptides fused to a heterologous peptide/polypeptide include SIRPαFc (aka TTI-621), a fusion protein consisting of a portion of the SIRPα receptor linked to an immunoglobulin Fc region forming a antibody-like molecule binds to CD47 on the tumor cells with high affinity and functions as a decoy receptor (developed by Trillium Therapeutics Inc., Mississauga, ON). Other useful CD47/SIRPα pathway peptide inhibitors include e.g., those derived from the C-terminal domain of TSP1 which bind to CD47 and perturb its interactions/signaling (e.g., as described in Frazier et al., UCSD Nature Molecule. 2010; and peptidomimetics derived therefrom), those that mimic CD47- or SIRPα-binding antibodies (e.g., an inhibitory peptide containing the following amino acid sequence CERVIGTGWVRC (SEQ ID NO:20); as described in Liu et al., J Immunol. 2004; 172(4):2578-2585; and analogs thereof), and the like.

Useful CD47/SIRPα pathway inhibitors include CD47/SIRPα inhibitory nucleic acids (such as e.g., CD47 morpholino oligonucleotides (e.g., as described in Isenberg et al., Ann Surg. 2008; 247(1):180-90; Isenberg et al., Circ Res. 2007; 100(5):712-20; Isenberg et al., Ann Surg. 2008; 247(5):860-8; and derivatives thereof), siRNA against human CD47 (e.g., as described in Toledano et al., PLoS One. 2013 Sep. 20; 8(9):e75595, and derivatives thereof); and the like.

Useful CD47/SIRPα pathway inhibitors include small molecule CD47/SIRPα inhibitors. Non-limiting examples of such include PSTx-23 and analogs thereof (such as e.g., those developed by Paradigm Shift Therapeutics (PSTx; Rockville, Md.).

Non-limiting examples of CD47/SIRPα pathway inhibitors include those described in e.g., Zhao et al., Proc Natl Acad Sci USA. 2011; 108(45):18342-7; Sagawa et al., Cancer Sci. 2011; 102(6):1208-15; Majeti et al., Cell. 2009; 138(2):286-99; Willingham et al., Proc Natl Acad Sci USA. 2012; 109(17):6662-7; van Ravenswaay Claasen et al., Gynecol Oncol. 1994; 52(2):199-206; Chao et al., Cancer Res. 2011 Feb. 15; 71(4):1374-84; Ide et al., Proc Natl Acad Sci USA. 2007; 104(12):5062-6; Fortin et al., J Exp Med. 2009; 206(9):1995-2011; Kikuchi et al., Leuk Res. 2005; 29(4):445-50; Soto-Pantoja et al., Expert Opin Ther Targets. 2013; 17(1): 89-103; US Patent Application Pub. Nos. 20070111238, 20120225073, 20130011401, 20130189253, 20140065169, 20140161825, 20140296477, 20140303354, 20140369924, 20150071905, 20150238604, 20150266942, 20150329616, 20150353642, 20150376288, 20160008429, 20160009815, 20160045532, 20160060342, 20160069898, 20160137734, 20160144009, 20160177276, 20160251435, 20160257751, 20160304609, 20160319256, 20160333093, U.S. Pat. Nos. 7,282,556; 8,562,997; 8,758,750; 8,759,025; 8,951,527; 9,017,675; 9,045,541; 9,382,320; 9,518,117; 9,527,901; PCT Pub. Nos. WO 2014094122 and WO 2013109752; the disclosures of which are incorporated herein by reference in their entirety.

CD47/SIRPα inhibitors may be administered to a subject in an effective amount, where an effective amount of a CD47/SIRPα inhibitor may be an amount effective to achieve the desired response and/or treat the individual. Effective amounts of CD47/SIRPα inhibitors may be determined empirically, e.g., through one or more clinical trials, inferred from one or more pharmacological studies and/or preclinical animal studies, or other means.

In certain embodiments, an effective amount of a CD47/SIRPα inhibitor for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a CD47/SIRPα inhibitor may be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

As will be readily understood, the methods of treating described herein may, in some instances, be combined with or performed before, during or after one or more conventional treatments. For example, in the case of oncology, the methods described herein may, in some instances, be combined with or performed before, during or after a conventional cancer therapy including but not limited to e.g., conventional chemotherapy, conventional radiation therapy, conventional immunotherapy, surgery, etc.

In some instances, as summarized above, the methods described herein may be used before or after a conventional therapy. For example, the methods described herein may be used as an adjuvant therapy, e.g., after a subject has seen improvement from a conventional therapy, or may be used when a subject has not responded to a conventional therapy. For example, the provided methods may be employed before, during or after a subject has been treated with conventional chemotherapy, including e.g., a chemotherapy regimen that includes one or more of prednisone, cytoxan, cisplatin, vincristine, and the like. In some instances, the subject may have not responded to prior treatment with a conventional chemotherapeutic, including e.g., prednisone, cytoxan, cisplatin, vincristine, and the like. In some instances, the methods described herein may be used prior to an additional therapy, e.g., to prepare a subject for an additional therapy, e.g., a conventional therapy as described herein.

In some instances, the provided methods may be used without any additional conventional therapy including e.g., where the method described herein is the sole method used to treat the subject. For example, in the case of oncology, the methods described herein may, in some instances, be the sole method used to treat the subject for a cancer.

In some embodiments, the provided methods may include administration of one or more conventional agents along with one or more of the therapeutic agents described herein. Such conventional agents may be “co-administered”. Agents useful in co-administration as described herein are agents used in conventional treatment of one or more of the cancers described herein. Such agents include but are not limited to e.g., conventional agents for treating cancer (e.g., chemotherapy agents), hormone agents (e.g., hormone therapy agents conventionally used for treating breast cancer, hormone therapy agents conventionally used for treating prostate cancer, etc.), agents for treating side effects of cancer therapy (e.g., analgesics, anti-nausea agents (e.g., corticosteroids, serotonin antagonists, dopamine antagonists, NK-1 inhibitors, cannabinoids, motion-sickness agents, sedatives, histamine H2-receptor antagonists, etc.), anti-neutropenia agents (e.g., antibiotics, antifungals, granulocyte colony-stimulating factors (G-CSFs), etc.)) agents for treating symptoms of a particular cancer or tumor, and the like. Additional agents useful in co-administration also include agents useful in treating or preventing conditions associated with radiation therapy.

Screening Methods

As summarized above, methods are also provided for identifying therapeutic agents that are effective during cancer cell MYC-regulated immune evasion. Such methods may be generally referred to as methods of screening. Methods are also provided which include treating a subject, as described above, through administering to the subject a therapeutic agent identified using such screening methods. Accordingly, therapeutic agents of the present disclosure include those agents identified as effective during MYC-regulated immune evasion of a cancer cell.

Methods of identifying a therapeutic agent that is effective during MYC-regulated immune evasion of a cancer cell will vary. Such methods may include contacting two different tumor cell lines conditionally expressing MYC with one or more candidate agents. For example, a method may include contacting a first tumor cell line conditionally expressing a MYC oncogene and a second tumor cell line conditionally expressing a MYC oncogene with a candidate therapeutic agent, where the second tumor cell line differs from the first. In some instances, the second tumor cell line may differ from the first in its expression of one or more immune checkpoint proteins. For example, the second tumor cell line may constitutively express one or more immune checkpoint proteins, including e.g., where the one or more immune checkpoint proteins are expressed in a MYC-independent manner.

Accordingly, the provided screening methods may include parallel or sequential assays of two substantially similar conditionally MYC expressing tumor cell lines that differ in their expression of one or more immune checkpoint proteins. Without being bound by theory, the provided screening methods incorporate the discovery that MYC regulates the expression of immune checkpoint proteins in tumor cells. Thus, in some embodiments, the provided methods involve screening candidate agents in two similar tumor cell lines, where, immune checkpoint protein expression is regulated by MYC in the first, and in the second, immune checkpoint protein expression is constitutive. Using this approach candidate agents that, e.g., affect the ability of MYC to induce immune checkpoint protein expression or other aspects of MYC-driven immune cell evasion, and candidate agents that are ineffective or act independently of MYC and/or the MYC-driven immune cell evasion pathway can be differentiated. For example, such methods may identify a candidate agent as a therapeutic agent that is effective during MYC-regulated immune evasion, where the agent produces a desired effect in a first tumor cell line (e.g., where immune checkpoint protein expression remains MYC-driven) but does not produce the effect in a second tumor cell line (e.g., where immune checkpoint protein expression is constitutive, i.e., not MYC driven). A “desired effect” may be a cellular behavior or cellular parameter, as described in more detail below. When identifying a candidate agent as one that is effective during MYC-regulated immune evasion, such cellular behavior(s) or cellular parameter(s) may be increased or reduced in one cell line as compared to another, depending on the context analyzed.

The term “tumor cell lines”, as used herein, is generally used to refer to neoplastic cell lines broadly and is not limiting to cell lines derived from solid tumors. As such, tumor cell lines utilized in the provided methods may be derived from any convenient source and established for essentially any neoplasia, including solid tumors and blood cancers. Thus, tumor cell lines utilized in the subject methods may include neoplastic cell lines generated from any cancer, including e.g., those cancers identified herein in relationship to other (i.e., non-screening) methods. In some instances, the tumor cell lines of the provided screening methods may be generated from a blood cancer (e.g., a lymphoma, a leukemia, etc.), a carcinoma, a melanoma, or the like. In some instances, the tumor cell lines are generated from an acute lymphoblastic leukemia. In some instances, the tumor cell lines are generated from a hepatocellular carcinoma, non-small cell lung carcinoma, renal cell carcinoma, colorectal carcinoma, or the like.

As summarized above, in some embodiments, a cell line employed in the methods may constitutively express an immune checkpoint protein independently of MYC. Such a protein may be referred to as a “MYC-independent constitutively expressed immune checkpoint protein” and may be expressed from the endogenous locus of the immune checkpoint protein of an introduced heterologous nucleic acid encoding the immune checkpoint protein. Through constitutive expression of the immune checkpoint protein the regulation of MYC and the immune checkpoint protein is uncoupled. Any convenient immune checkpoint protein may be employed as a MYC-independent constitutively expressed immune checkpoint protein and conventional methods for constitutively expressing a desired protein may be employed (such as e.g., configuring the immune checkpoint protein coding sequence such that it is operably linked to a constitutive promoter). In some instances, the MYC-independent constitutively expressed immune checkpoint protein is CD47. In some instances, the MYC-independent constitutively expressed immune checkpoint protein is PD-L1. In some instances, a cell line may be configured to constitutively express both CD47 and PD-L1 in a MYC independent manner.

Conditional control of the expression of MYC may be performed using any convenient method of conditional expression, either reversible or irreversible. For example, in some instances, a MYC coding sequence may be placed under the control of (i.e., may be operably linked to) an inducible or conditionally active promoter. Conventional conditional expression systems may be employed including e.g., where the conditional expression of MYC is controlled by a tetracycline-based system, a tamoxifen-based system or the like. As such, conditional expression of MYC may be controlled (i.e., activated or repressed) by contacting the cells with an agent that activates or represses the expression of MYC in the cell lines, such as but not limited to e.g., tetracycline or one of its derivatives (e.g. doxycycline), tamoxifen or one of its derivatives (e.g., 4-hydroxytamoxifen), and the like.

Contacting of the tumor cell lines with the candidate agent(s) may be performed in vitro or in vivo. For example, in some instances, the tumor cell lines may be cultured, including e.g., in a two-dimensional or a three-dimensional cell culture environment (e.g., in a well, dish, flask, etc.) and the agent(s) may be added to the culture (e.g., added to the culture medium). In some instances, the tumor cell lines may be introduced, including e.g., transplanted (e.g., grafted or xeno-grafted) into a host animal (e.g., a rodent such as a mouse or a rat, or a non-human primate or other mammal) and the agent(s) may be administered to the host animal (e.g., through any convenient route of administration, including e.g., those described herein).

The provided methods may include a step of determining the effect of contacting the tumor cell line(s), whether performed in vitro or in vivo, with the candidate agent. In some embodiments, the determining will include comparing a cellular behavior or cell parameter of two cell lines where both cell lines conditionally express MYC but only one cell line constitutively expresses one or more immune checkpoint proteins. For example, the two cell lines may be compared in both a “MYC ON” state where the conditional expression of MYC is activated or de-repressed and a “MYC OFF” state where the conditional expression of MYC is deactivated or repressed. Any convenient parameter or behavior of the individual cells or the cells as a population may be analyzed, including e.g., cell growth, cell division, cell line expansion, cell viability, cell line survival, gene expression (e.g., immune checkpoint gene expression), etc. In some instances, one or more parameters or behaviors may be quantified.

A wide variety of assays may be used for screening purposes, including immunoassays for protein binding; determination of cell growth, differentiation and functional activity; production of factors; and the like. Screening assays may utilize cells, proteins, polynucleotides, etc. In some embodiments, isolated polypeptides corresponding to a candidate or combination of candidates of the present invention are useful in drug screening assays.

In screening assays for biologically active agents, tumor cell lines conditionally expressing a MYC oncogene, are contacted with the agent of interest, and the effect of the agent assessed by monitoring output parameters on cells (also referred to as cellular behaviors), such as expression of markers, cell viability, and the like. The cells may be freshly isolated, cultured, genetically altered, or transplanted as xenografts, and the like. The cells may be environmentally induced variants of clonal cultures: e.g. split into independent cultures and grown under distinct conditions, for example with or without drugs; in the presence or absence of cytokines or combinations thereof. The manner in which cells respond to an agent, particularly a pharmacologic agent, including the timing of responses, is an important reflection of the physiologic state of the cell.

Parameters are quantifiable or qualitatively assessable components of cells, including components that can be accurately measured or easily distinguished, e.g., in a high throughput system. A parameter can be any cell population feature or cell component or cell product including e.g., cell survival, cell line expansion, a cell surface determinant, a receptor, a protein or conformational or posttranslational modification thereof, a lipid, a carbohydrate, an organic or inorganic molecule, a nucleic acid, e.g. mRNA, DNA, etc., or a portion derived from such a cell component or combinations thereof. While many parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. One aspect of some embodiments is to evaluate candidate drugs, including biological activity assays, toxicity testing; and the like.

In addition to complex biological agents, candidate agents may include organic molecules comprising functional groups necessary for structural interactions, particularly hydrogen bonding, and typically include at least an amine, carbonyl, hydroxyl or carboxyl group, frequently at least two of the functional chemical groups. The candidate agents may comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g., ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection. In some instances, from about 0.01 to 1 ml of a biological sample may be sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

In some embodiments, agents are screened for biological activity by adding the agent to at least two, i.e., a plurality of cell samples, including e.g., a MYC expressing cell line and a cell line that expresses both MYC and an immune checkpoint protein, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

Therapeutic agents identified in the provided screening methods may have a wide variety of mechanisms of action to produce the identified effect. For example, in some instances, a therapeutic agent identified as overcoming MYC-regulated immune evasion may inhibit MYC-induced expression of one or more immune checkpoint genes, including e.g., where the agent directly or indirectly inhibits MYC-induced expression of one or more immune checkpoint genes. In another example, the therapeutic agent may overcome MYC-regulated immune evasion by inhibiting MYC-induced expression of CD47. In another example, the therapeutic agent may overcome MYC-regulated immune evasion by inhibiting MYC-induced expression of PD-L1. In another example, the therapeutic agent may overcome MYC-regulated immune evasion by inhibiting MYC-induced expression of CD47 and PD-L1. It will be readily understood that mechanisms of action of identified candidate agents may vary.

Pharmaceutical Compositions

The provided compositions (e.g., those including one or more therapeutic agents, including previously described agents and newly identified therapeutic candidates) can be supplied in the form of a pharmaceutical composition. Any suitable pharmaceutical composition may be employed, described in more detail below. As such, in some instances, methods of the present disclosure may include administering an inhibitor in a composition comprising an excipient (e.g., an isotonic excipient) prepared under sufficiently sterile conditions for administration to a mammal, e.g., a human.

Administration of an inhibitor to a subject, as described herein, may be performed employing various routes of administration. The route of administration may be selected according to a variety of factors including, but not necessarily limited to, the condition to be treated, the formulation and/or device used, the patient to be treated, and the like. Routes of administration useful in the disclosed methods include but are not limited to oral and parenteral routes, such as intravenous (iv), intraperitoneal (ip), rectal, topical, ophthalmic, nasal, and transdermal. Formulations for these dosage forms are described herein.

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (host) being treated.

Therapeutically effective doses of a subject compound or pharmaceutical composition can be determined by one of skill in the art, with a goal of achieving local (e.g., tissue) concentrations that are at least as high as the IC50 of an applicable compound disclosed herein.

The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, Md. 20857; (available at www(dot)fda(dot)gov/cder/guidance/index(dot)htm, the disclosure of which is incorporated herein by reference).

Conversion of Animal Doses to Human Equivalent Doses
Based on Body Surface Area

| | To Convert Animal | To Convert Animal Dose in mg/ | |
|---|---|---|---|
| | Dose in mg/kg to | kg to HED^{a} in mg/kg, Either: | |
| | Dose in mg/m², | Divide | Multiply |
| Species | Multiply by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| monkey | | | |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)0.33.
^{b}This km value is provided for reference only since healthy children will rarely be volunteers for phase 1 trials.
^{c}For example, cynomolgus, rhesus, and stumptail.

A pharmaceutical composition comprising a subject compound (i.e., an inhibitory agent or a combination thereof) may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, tablet, pill, pellet, capsule, powder, syrup, elixir or any other dosage form suitable for administration.

A subject compound may be administered to the host using any convenient means capable of resulting in the desired reduction in disease condition or symptom. Thus, a subject compound can be incorporated into a variety of formulations for therapeutic administration. More particularly, a subject compound can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the subject compounds can be formulated for use in human or veterinary medicine. Particular formulations of a disclosed pharmaceutical composition may depend, for example, on the mode of administration and/or on the location of the infection to be treated. In some embodiments, formulations include a pharmaceutically acceptable carrier in addition to at least one active ingredient, such as a subject compound. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A subject compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A subject compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

In some embodiments, a subject compound can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

A subject compound can be utilized in aerosol formulation to be administered via inhalation. A subject compound can be formulated into pressurized acceptable propellants such as dichlorodifluoromethane, propane, nitrogen and the like.

Furthermore, a subject compound can be made into suppositories by mixing with a variety of bases such as emulsifying bases or water-soluble bases. A subject compound can be administered rectally via a suppository. The suppository can include vehicles such as cocoa butter, carbowaxes and polyethylene glycols, which melt at body temperature, yet are solidified at room temperature.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions comprising a subject compound may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

### Cell Lines, Transgenic Animals and Kits

As summarized above, also provided are cell lines, transgenic animals and kits for use in the subject methods. The subject cell lines include neoplastic cell lines (e.g., tumor cell lines, cancer cell lines, etc.). In some instances, the subject cell lines may include neoplasms derived from a mammalian neoplasia such as e.g., a human neoplasia, a mouse neoplasia, a rat neoplasia, a primate neoplasia, or the like. The subject transgenic animals include non-human transgenic animals, such as transgenic non-human mammals, e.g., transgenic non-human rodents (e.g., mice, rats, etc.), transgenic non-human primates, and the like. The subject kits may include any combination of components (e.g., therapeutic agents, reagents, cell lines, etc.) for performing the subject methods. Provided kits include kits useful in methods of treating a subject for a neoplasm and/or methods of identifying a neoplasm as immune evasive and/or likely to be responsive to an immune checkpoint inhibitor. Also provided are kits for use in generating one or more of the described cell lines and/or transgenic animals.

As summarized above, the provided cell lines may include cancer cell lines or tumor cell lines. The terms “cancer cell lines” and “tumor cell lines”, as used herein, is generally used to refer to neoplastic cell lines broadly. The subject terms are not limiting to cell lines derived from solid tumors or specific cancers. As such, cancer and tumor cell lines utilized in the provided methods may be derived from any convenient source and established for essentially any neoplasia, including solid tumors and blood cancers. Thus, cancer and tumor cell lines utilized in the subject methods may include neoplastic cell lines generated from essentially any cancer, including e.g., those cancers identified herein in relationship to the above methods. In some instances, the cell lines of the provided screening methods may be generated from a blood cancer (e.g., a lymphoma, a leukemia, etc.), a carcinoma, a melanoma, or the like. In some instances, the tumor cell lines are generated from an acute lymphoblastic leukemia. In some instances, the cell lines are generated from a hepatocellular carcinoma, non-small cell lung carcinoma, renal cell carcinoma, colorectal carcinoma, or the like.

In some embodiments, the cell lines will include an inducible MYC locus, allowing for the inducible or conditional expression of MYC. Accordingly, at a minimum an inducible MYC locus will generally include a nucleic acid sequence encoding MYC operably linked to a inducible, or otherwise conditionally active, transcriptional response element. Transcriptional response elements include e.g., promoters and enhancers. As such, a transcriptional response element to which a MYC coding sequence may be operably linked in an inducible MYC locus may be an inducible promoter, which may or may not include one or more operably linked enhancer elements.

In some embodiments, the cell lines will include a constitutively active locus, allowing for continued expression of a desired coding sequence. For example, a cell line may include a constitutively active locus encoding one or more immune checkpoint proteins. Such constitutively active loci encoding one or more immune checkpoint proteins may be MYC-independent such that the expression of MYC does not affect the expression of the encoded one or more immune checkpoint proteins. Accordingly, in some embodiments, cell lines may include a MYC-independent, constitutively active immune checkpoint encoding locus. At a minimum such a locus will include an immune checkpoint protein encoding nucleic acid sequence operably linked to a constitutively active transcriptional response element, e.g., a constitutively active promoter, that may or may not include one or more operably linked enhancer elements. In some instances, the immune checkpoint protein encoding nucleic acid sequence employed encodes CD47. In some instances, the immune checkpoint protein encoding nucleic acid sequence employed encodes PD-L1. In some instances, the immune checkpoint protein encoding nucleic acid sequence employed encodes both CD47 and PD-L1. In some instances, two separate immune checkpoint protein encoding nucleic acid sequences may be employed, e.g., one encoding CD47 and one encoding PD-L1, e.g., each operably linked to the same or different constitutively active transcriptional response elements.

Suitable transcriptional response elements will vary depending on the context in which they are employed. Suitable promoter and enhancer elements include e.g., those conventionally employed for expression in a eukaryotic cell, including but are not limited to e.g., cytomegalovirus immediate early promoter; herpes simplex virus thymidine kinase promoter; early and late SV40 promoters; promoter present in long terminal repeats from a retrovirus; mouse metallothionein-I promoter; and the like.

Any convenient and appropriate inducible and/or reversible promoter, including reversible/inducible promoters may be employed. Such promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., can be performed. Such promoters, and systems based on such promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated (e.g., tetracycline-dependent) promoters, (e.g., promoter systems including TetActivators, TetON, TetOFF, etc.), steroid regulated promoters (e.g., rat glucocorticoid receptor promoter systems, human estrogen receptor-based promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some instances, the locus or construct or transgene containing the suitable promoter is irreversibly switched through the induction of an inducible system. Suitable systems for induction of an irreversible switch include, e.g., those that involve induction of an irreversible switch and make use of a Cre-lox-mediated recombination (see, e.g., Fuhrmann-Benzakein, et al., PNAS (2000) 28:e99, the disclosure of which is incorporated herein by reference). Any suitable combination of recombinase, endonuclease, ligase, recombination sites, etc. may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, Mass.), the disclosures of which are incorporated herein by reference.

Useful transcriptional response elements in the subject cells include but are not limited to e.g., a transcriptional response element that is a tetracycline-dependent promoter. For example, the tetracycline-dependent promoter may be operably linked to a MYC encoding nucleic acid such that when active (e.g., through the presence or absence of tetracycline of a derivative thereof) MYC is conditionally expressed.

Useful transcriptional response elements in the subject cells include but are not limited to e.g., a transcriptional response element that is a GAL4 responsive upstream activating sequence (UAS). For example, the GAL4 responsive UAS may be operably linked to a MYC encoding nucleic acid such that when active (e.g., through the presence of GAL4) MYC is conditionally expressed. Correspondingly, the expression of GAL4 may be controlled by a promoter (e.g., an inducible or conditional promoter) operably linked to a GAL4 coding sequence.

An inducible MYC locus may be reversibly or irreversibly inducible as desired. For example, in some instances, an inducible or conditional MYC may be configured such that, upon a recombination event, the MYC encoding locus is irreversibly switched “ON” or “OFF”, e.g., through the use of a conditional genetic stop sequence. In some embodiments, a conditional genetic stop sequence may be configured between a transcriptional response element and a MYC encoding nucleic acid sequence and flanked by recombination sites (e.g., “floxed”) such that upon a recombination event (e.g., mediated by Cre) the stop sequence is removed and MYC is controlled by the transcriptional response element (and may be expressed or repressed accordingly). In some instances, the recombination activity of a Cre recombinase may be controlled using a tamoxifen-based system, e.g., by fusing the Cre to a portion of the estrogen receptor (Cre-ER). An ordinary skilled artisan will readily understand how the described cells may be combined with and adapted to systems for reversible and/or irreversible conditional and/or inducible expression of desired coding sequences.

As summarized above, the provided non-human transgenic animals include transgenic non-human mammals, e.g., transgenic non-human rodents (e.g., mice, rats, etc.), transgenic non-human primates. Such animals will generally be permanently or transiently genetically modified with one or more heterologous nucleic acid sequences to generate an inducible MYC expressing locus and/or a constitutive immune checkpoint expressing locus in the animal. Any convenient method of genetic manipulation may be employed.

The provided transgenic animals may utilize endogenous or exogenous coding sequencing, e.g., MYC coding sequences, CD47 coding sequences, PD-L1 coding sequences, etc. For example, a heterologous transcriptional response element may be genetically inserted such that it is operably linked to an endogenous coding sequence. Alternatively, an exogenous coding sequence may be introduced already operably linked to a heterologous transcriptional response element. Where exogenous coding sequences are introduced into a subject animal, the corresponding endogenous coding sequence may or may not be replaced or otherwise genetically disrupted as desired.

In some embodiments, the animals will include an inducible MYC locus, allowing for the inducible or conditional expression of MYC. Accordingly, at a minimum an inducible MYC locus will generally include a nucleic acid sequence encoding MYC operably linked to an inducible, or otherwise conditionally active, transcriptional response element. Transcriptional response elements include e.g., promoters and enhancers. As such, a transcriptional response element to which a MYC coding sequence may be operably linked in an inducible MYC locus may be an inducible promoter, which may or may not include one or more operably linked enhancer elements.

In some embodiments, the animals will include a constitutively active locus, allowing for continued expression of a desired coding sequence. For example, an animal may include a constitutively active locus encoding one or more immune checkpoint proteins. Such constitutively active loci encoding one or more immune checkpoint proteins may be MYC-independent such that the expression of MYC does not affect the expression of the encoded one or more immune checkpoint proteins. Accordingly, in some embodiments, animals may include a MYC-independent, constitutively active immune checkpoint encoding locus. At a minimum such a locus will include an immune checkpoint protein encoding nucleic acid sequence operably linked to a constitutively active transcriptional response element, e.g., a constitutively active promoter, that may or may not include one or more operably linked enhancer elements. In some instances, the immune checkpoint protein encoding nucleic acid sequence employed encodes CD47. In some instances, the immune checkpoint protein encoding nucleic acid sequence employed encodes PD-L1. In some instances, the immune checkpoint protein encoding nucleic acid sequence employed encodes both CD47 and PD-L1. In some instances, two separate immune checkpoint protein encoding nucleic acid sequences may be employed, e.g., one encoding CD47 and one encoding PD-L1, e.g., each operably linked to the same or different constitutively active transcriptional response elements.

Provided kits include those containing one or more reagents for identifying whether a cancer will be responsive to an immune checkpoint inhibitor. Such kits may further include, e.g., in addition to one or more reagents for identifying whether the cancer will be responsive to an immune checkpoint inhibitor, one or more therapeutic agents (e.g., an immune checkpoint inhibitor) to be administered to a subject when the cancer is identified as likely to be responsive.

Provided kits include those containing one or more reagents for determining whether a cellular sample from a subject is an immune-evasive cancer or whether a cellular sample of a cancer from a subject is immune-evasive. Such kits may include, for example, reagents useful for determining whether a cellular sample overexpresses CD47, PD-L1 or both. Such kits may further include, e.g., in addition to one or more reagents for determining whether a cellular sample from a subject is an immune-evasive cancer or whether a cellular sample of a cancer from a subject is immune-evasive, one or more therapeutic agents (e.g., a MYC inhibitor) to be administered to a subject when the cancer is identified as immune evasive and/or as overexpressing CD47, PD-L1 or both. In some instances, such kits may include one or more therapeutic agents, e.g., a bromodomain and extra-terminal (BET) inhibitor, including but not limited to e.g., JQ1 or an analog thereof. In some instances, the subject kits may further include one or more conventional cancer therapeutics, e.g., one or more chemotherapeutics.

In addition to the above components, the subject kits and/or cell lines may further include (in certain embodiments) instructions for practicing the subject methods. These instructions may be present in the subject kits or provided with the subject cell lines in a variety of forms, one or more of which may be present in the kit or provided with the subject cell lines. One form in which these instructions may be present is as printed information on a suitable medium or substrate, e.g., a piece or pieces of paper on which the information is printed, in the packaging of the kit or cell line(s), in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example: Myc Regulates the Antitumor Immune Response Through CD47 and PD-L1

Materials and Methods

Transgenic Mice

Animals were identically raised and housed; immunocompromised mice were maintained on Bactrim chow. The Tet-off system transgenic lines used for conditional MYC expression and oncogene addiction have been previously described (Felsher et al. 1999, Shachaf et al. 2004, Weinstein et al. 2008). Ep-tTA/tet-O-MYC mice develop lymphoma/leukemia (MYC T-ALL) and LAP-tTA/tet-O-MYC mice develop HCC (MYC HCC). RAG1 KO mice on an FVB background were generously provided. Genotyping was performed using genomic DNA from animal tails. For transgenic primary tumors, a minimum of 3 mice were used per treatment condition group. Male mice were used for all transgenic mouse experiments and tumors arose at approximately 2-3 months of age.

Cell Culture

Hematopoietic tumor cell lines (MYC T-ALL 4188 cells derived from the transgenic mouse model, human P493-6, human T-ALL Jurkat, and human T-ALL CCRF-CEM) were grown in RPMI 1640 supplemented with 10% FBS, 1% penicillin/streptomycin, 50 μM 2-mercaptoethanol at 37° C. in 5% CO2. Solid human tumor cell lines (H1299, SKMEL28), mouse MYC HCC, and human HEPG2 were grown in DMEM supplemented with 10% FBS and 1% penicillin/streptomycin at 37° C. in 5% CO2. To turn off expression of the MYC transgene, cells were treated with 20 ng/mL Dox. Primary human T-ALL cells were obtained from four deidentified patient samples and were cultured as previously described (Bruserud et al. 2003). Briefly, cells were cultured in StemSpan H3000 (StemCell) supplemented with human stem cell factor (50 ng/ml), human IL-7 (20 ng/ml), human IL-3 (20 ng/ml), and human FLT3 ligand (20 ng/ml) (Peprotech). Human cell lines were purchased from ATCC.

Overexpressions and Knockdowns

For forced expression of CD47 and PD-L1, cDNAs were cloned into MSCV-Hygro or MSCVPuro backbones. The plasmids (CD47, PD-L1, or empty control MSCV) were transfected into 293T cells and virus-containing medium supernatants were used to infect tumor cells using polybrene methodology. Infected cells were selected, expanded, forced expression confirmed, and prepared for injection. For forced expression, one cohort was collected at day 0 and day 4 after turning MYC “off” with doxycycline. These tissues were used for gene expression analysis and immunostaining. Another, separate cohort was followed for survival analysis.

shRNA-mediated knockdowns were performed with the pLKO.1 lentiviral vector containing either control, CD47, or PD-L1-specific shRNA. shRNA-mediated knockdown of human MYC was performed with the pLKO.1 lentiviral vector containing a scrambled control or MYC shRNA (Sigma). PD-L1 shRNA on a pLKO.1-puro backbone was purchased from Sigma and CD47 shRNA (Sigma) was cloned into a pLKO.1-Hygro backbone (Addgene). The plasmids were transfected into packaging cells and virus-containing medium supernatants were used to infect tumor cells using polybrene methodology. For conditional knockdowns, stable cell lines were generated and the time course following MYC inactivation with 20 ng/mL Dox is indicated. For stable knockdowns, the time course is indicated as the time post-viral infection.

Tumor Growth Assays

10×10⁶ tumor cells were injected subcutaneously in a volume of 100 ul into 4-6 week old female FVB animals and were allowed to form tumors of 1.5 cm in diameter. At that time, mice were either euthanized and tissues collected or treated with Dox in their drinking water (100 μg/mL final concentration).

Reconstitutions of RAG1 KO Mice

4-6 week old female FVB RAG1 KO mice were injected intravenously with 4×10⁶ purified CD4+ T cells that had been magnetically enriched from CAG-luc-eGFP L2G85 mice (Miltenyi Biotec beads and columns). Tumor cells were injected one week after reconstitution.

In Vivo Bioluminescence Imaging

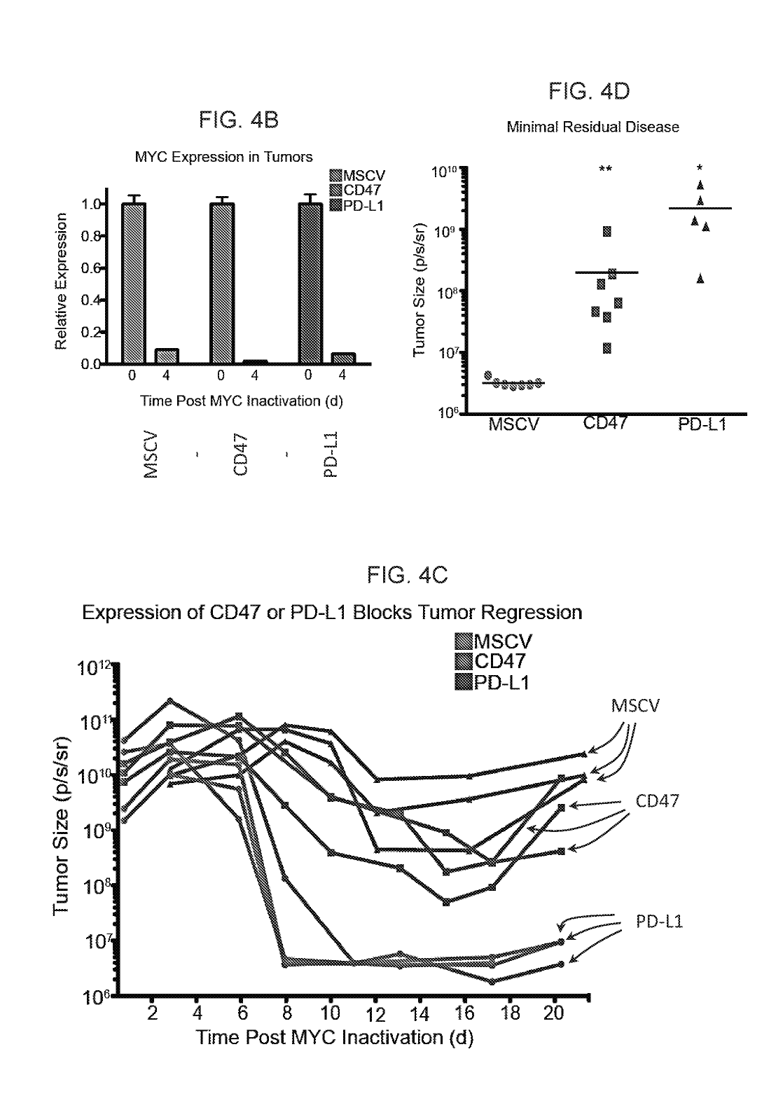

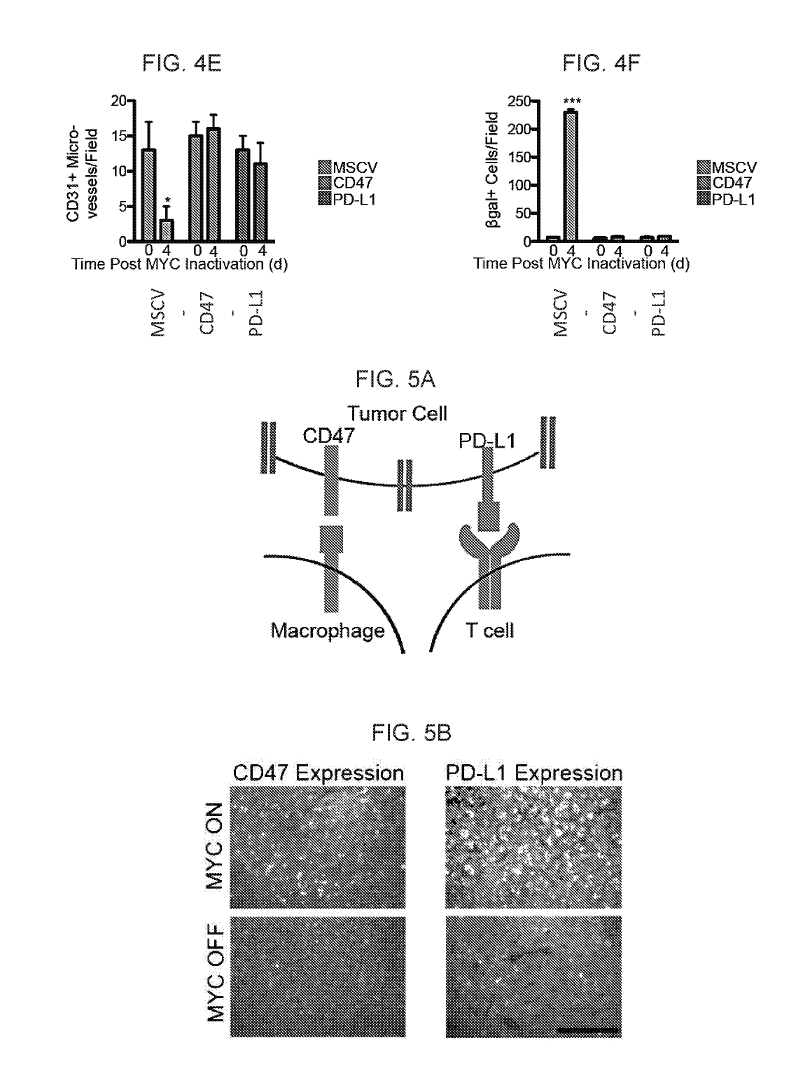

Bioluminescence imaging was performed as previously described (Rakhra et al. 2010). Briefly, cell lines (FIG. 4) were infected with pMSCVneo containing fLuc. For fLuc immune labeling, see Reconstitutions of RAG1 KO mice above. Mice were anesthetized with inhaled isoflorane/oxygen using the Xenogen XGI-8 5-port Gas Anesthesia System. d-luciferin (150 mg/kg) was injected intraperitoneally 10 min prior to imaging. Animals were placed into the light-tight chamber and were imaged with an IVIS-200 cooled CCD camera (Xenogen). Living Image (Xenogen) was used to collect and analyze data and generate pseudocolor images.

RNA Isolation, cDNA Preparation, and qPCR

All tissues and cells were flash-frozen in liquid nitrogen and stored at −80° C. until RNA preparation. RNA was isolated using the TRIzol Reagent (Invitrogen) according to manufacturer instructions. cDNA was synthesized using SuperScriptIII (ThermoFisher). qPCR was performed using specific primers (Table 51) and the SYBR Green qPCR Kit (Roche) in an Applied Biosystems Real-Time PCR System (Life Technologies) with QuantStudio12K Flex Software. Data was analyzed using the cycle threshold method (normalized to UBC). A minimum of 3 biological and 3 technical replicates were used for all qPCR experiments.

Lymphocyte Analysis and Flow Cytometry

Tissues were manually dissociated between microscope slides and strained to remove debris. Lysis buffer (0.15 M NH₄Cl, 10 mM KHCO3, 0.1 mM EDTA pH 7.3) was used to remove red blood cells. Cells were washed and resuspended in cold flow cytometry buffer (1% FBS, 0.1% sodium azide in PBS). Staining was performed according to manufacturer instructions with the indicated antibodies (eBioscience). Labeled cells were analyzed on a FACSCalibur (Becton Dickinson), and data was analyzed using FlowJo (Treestar). Each plot is representative of three or more animals. Annexin-V and 7-AAD were used according to manufacturer's protocol (eBioscience).

Immunohistochemistry and Immunofluorescence

Immunohistochemistry and immunofluorescence were performed as previously described (Rakhra et al. 2010). Briefly, senescence-associated β-galactosidase staining was performed on tissue sections that had been embedded in OCT freezing medium (Tissue Tek) and stored at −80° C. Tissue sections were warmed to room temperature, fixed in 0.5% glutaraldehyde in PBS, and washed in PBS pH 5.5. Sections were stained for 8 h in a solution containing 250 mM potassium ferricyanide, potassium ferrocyanide, and MgCl2 in PBS pH 5.5, mounted in 50% glycerol/PBS and promptly imaged.

CD31 staining was also performed on frozen sections. Briefly, slides were thawed, fixed in acetone, blocked in Dako serum-free blocking reagent, incubated in rat anti-mouse CD31 (1:50, BD Pharmingen) for 1.5 hours at room temperature, incubated with anti-rat biotinylated IgG (1:300) and Cy3-streptavidin (1:300) for 30 min at room temperature), and counterstained with DAPI.

CC3, PH3 (Cell Signaling), F4/80 (Invitrogen), CD69 (AbCam), CD47, and PD-L1 (R&D Systems) primary antibodies were used according to manufacturer recommendations. Tumors were fixed in 4% paraformaldehyde, embedded in paraffin, and sectioned. Sections were stained as previously described (4). CD47, PD-L1, CC3, and PH3 stainings were counterstained with DAPI and CD69 and F4/80 stainings were counterstained with hematoxylin.

Pictures were taken with 20-40× objectives on a Leica DM16000 microscope with LASAF software. At least 5 random fields per section and at least 3 tumors per experimental condition were imaged.

ChIP

ChIP-sequencing reads were aligned to the murine mm9 or the human hg19 reference genome using Bowtie v0.12.7 and binding traces were obtained using MACS v1.4.2. To allow comparisons across samples, all files were normalized to the same number of mappable reads. Binding profiles were visualized using the Integrated Genome Browser software (Nicol et al. 2009).

Nuclear Run-on

The nuclear run-on assay was performed as previously reported (Fan et al. 2010, Fan et al. 2012, Ji et al. 2011). Briefly, P493-6 nuclei were isolated using cell lysis buffer. The nascent RNA was labeled with Biotin-UTP. Total nuclear RNA was purified with the Qiagen RNeasy kit. Capture of Biotin-labeled nascent RNA was followed by cDNA synthesis, cRNA synthesis, and cRNA purification (Ambion TotalPrep RNA Amplification) before hybridization to the Affymetrix microarray platform.

Correlation of Gene Expression in Human Tumors

Correlation of MYC expression with immune-related surface receptors was determined using the R2 platform (R2: Genomics Analysis and Visualization Platform (http(colon)//r2(dot)amc(dot)nl)) with datasets for human liver (Wurmbach et al. 2007), kidney (Kort et al. 2008), and colon tumors (Julien et al. 2012). For each gene, one highly expressed probe was used to determine the Pearsons correlation coefficients and P-values.

Statistics

Differences between groups were analyzed using one-way ANOVA with a Tukey's post hoc test. A logrank test was performed for survival analysis (FIG. 4A). P<0.05 was considered to be significant and is indicated by one asterisk (*). P<0.01 is indicated by two asterisks (**). P<0.001 is indicated by three asterisks (***). Graphs are presented as the mean±SEM. Analyses were performed with GraphPad Prism, version 5 (GraphPad Software).

Results

The MYC oncogene codes for a transcription factor that is overexpressed in many human cancers. Here we show that MYC regulates the expression of two immune checkpoint proteins on the tumor cell surface, the innate immune regulator, CD47 (Cluster of Differentiation 47) and the adaptive immune checkpoint, PD-L1 (programmed death-ligand 1). Suppression of MYC in mouse tumors and human tumor cells caused a reduction in the levels of CD47 and PD-L1 mRNA and protein. MYC was found to bind directly to the promoters of the CD47 and PD-L1 genes. MYC inactivation in mouse tumors downregulated CD47 and PD-L1 expression and enhanced the anti-tumor immune response. In contrast, when MYC was inactivated in tumors with enforced expression of CD47 or PD-L1, the immune response was suppressed and tumors continued to grow. Thus MYC appears to initiate and maintain tumorigenesis in part through the modulation of immune regulatory molecules.

MYC is a transcription factor that regulates the expression of a multitude of gene products involved in cell proliferation, growth, differentiation, and apoptosis (Felsher et al. 2009, Jain et al. 2002, Shachaf et al. 2004, Wu et al. 2007). The MYC gene is genetically activated and overexpressed in many human cancers (Felsher et al. 2009, Jain et al. 2002, Shachaf et al. 2004, Wu et al. 2007) and this overexpression has been causally linked to tumorigenesis (Sharma et al. 2006, Wang et al. 2006). Work with inducible transgenic mouse models has shown that growth of MYC-induced tumors is dependent on continuous expression of MYC (Felsher et al. 2009, Jain et al. 2002, Shachaf et al. 2004, Wu et al. 2007, Marinkovic et al. 2004, Hennighausen et al. 1995, Pelengaris et al. 2002, D'Cruz et al. 2001). For example, in the tetracycline-off mouse model (where MYC expression can be turned off by the addition of tetracycline or doxycycline), tumors grow only when MYC is “on.” When MYC is turned “off,” tumors regress.

MYC inactivation in mouse models results in tumor regression through the induction of proliferative arrest and apoptosis (Felsher et al. 2009, Jain et al. 2002, Shachaf et al. 2004, Marinkovic et al. 2004, Hennighausen et al. 1995, D'Cruz et al. 2001, Fisher et al. 2001, Huettner et al. 2000). We have demonstrated that complete tumor clearance following the inactivation of oncogenes, including MYC, requires the recruitment of CD4+ T cells and the secretion of Thrombospondin-1 (Rakra et al, 2010, Giuriato et al. 2006). Hence, a host-dependent immune response is required for sustained tumor regression. However, the mechanism by which oncogene inactivation elicits this immune response is unknown.

The host immune system generally serves as a barrier against tumor formation (Balkwill et al. 2001). Activation of the immune response can contribute to tumor regression (Rakhra et al. 2010, Gattinoni et al. 2006, Galon et al. 2014) through both adaptive and innate immune effectors (Shanker et al. 2007, Corthay et al. 2005, Qin et al. 2000). Programmed death-ligand 1 (or PD-L1, also known as CD274 and B7-H1) is a critical “don't find me” signal to the adaptive immune system (Parsa et al. 2007, Topalian et al. 2012, Tsushima et al. 2007), whereas CD47 is a critical “don't eat me” signal to the innate immune system as well as a regulator of the adaptive immune response (Jaiswal et al. 2009, Majeti et al. 2009) (FIG. 5A). These and similar molecules are often overexpressed on human tumors (Topalian et al. 2012, Majeti et al. 2009). Therapeutic suppression of PD-L1 and other immune checkpoint molecules elicit an immune response against tumors and recently this strategy has been translated to the clinic, with very encouraging results (Sharma et al. 2015, Chao et al. 2011, Woo et al. 2015, Galluzzi et al 2014).

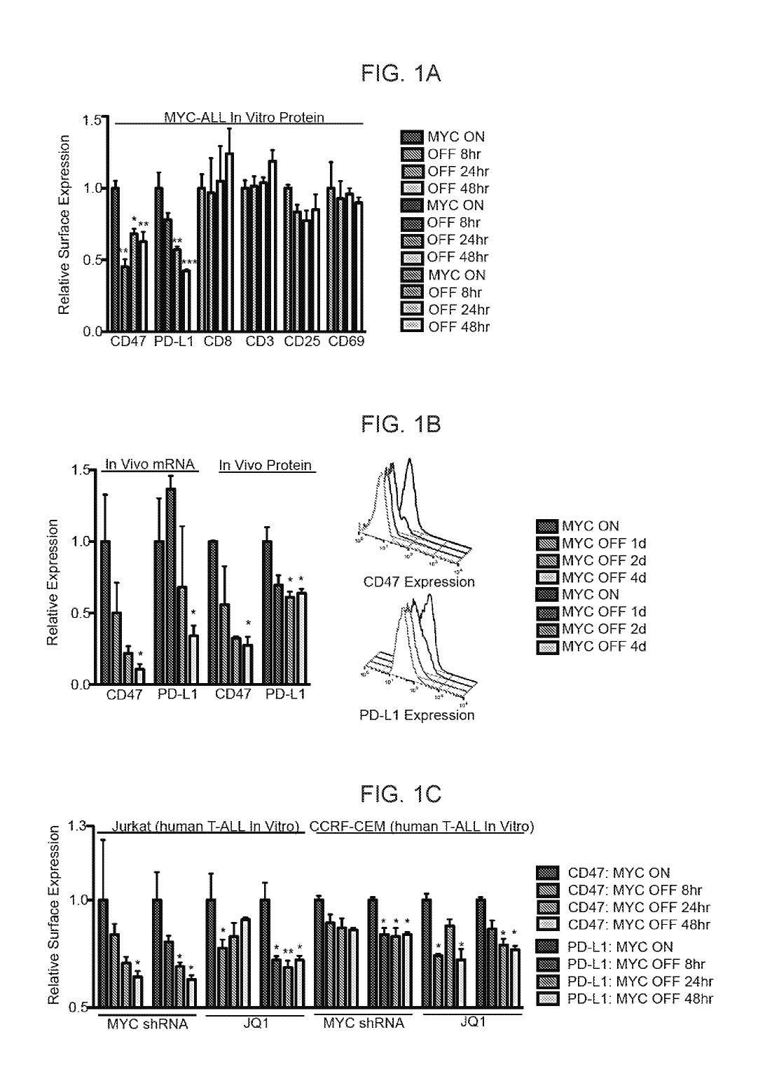

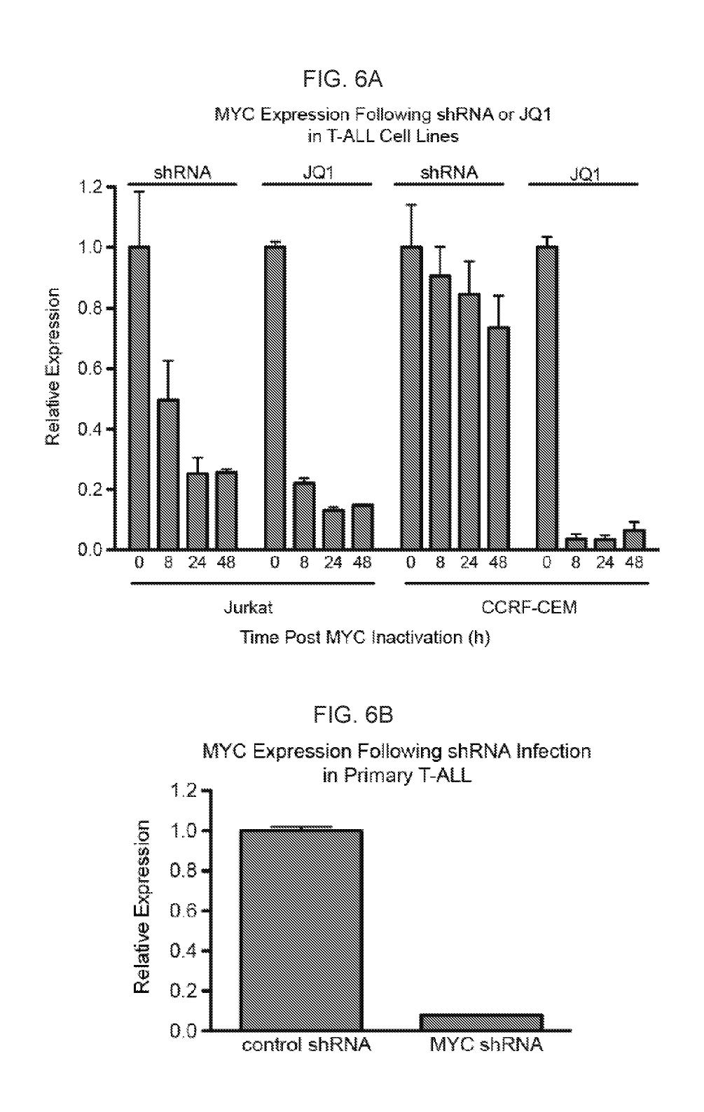

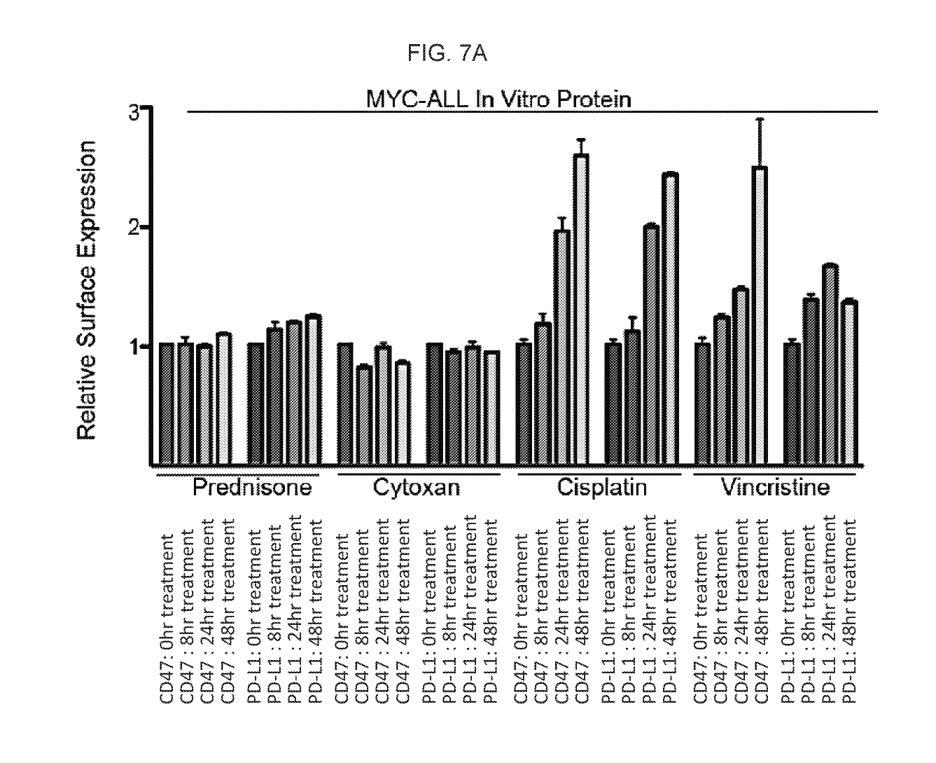

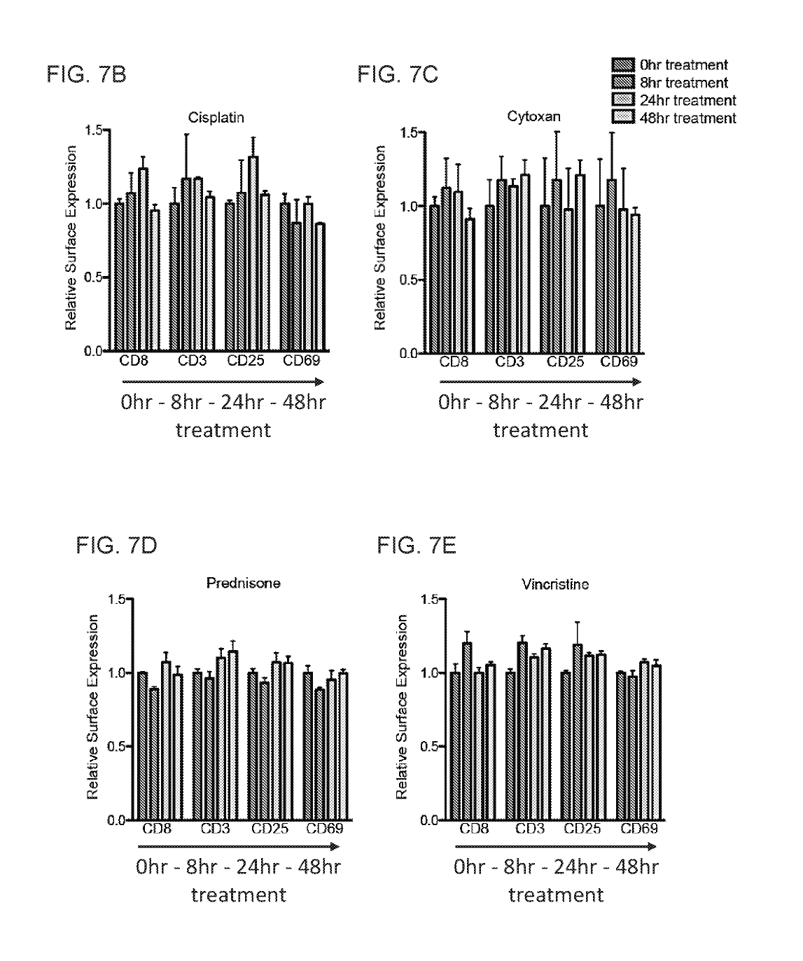

To explore whether and how MYC regulates the anti-tumor response, we examined its effect on the expression of CD47 and PD-L1 in the Tet-off transgenic mouse model of MYCinduced T cell acute lymphoblastic leukemia (MYC T-ALL). When MYC was “on,” both CD47 and PD-L1 were expressed. However, in vitro or in vivo MYC inactivation resulted in a rapid downregulation of CD47 and PD-L1, both at the mRNA level, as detected by quantitative realtime PCR (qPCR), and at the protein level, as detected by flow cytometry (FIGS. 1A-B) and immunofluorescence (FIG. 5B). Expression of other immune-related surface receptors was not affected by MYC inactivation (FIG. 1A). Consistent with these observations, suppression of MYC expression in the human T-ALL cell lines, CCRF-CEM and Jurkat, either by treatment with a MYC-targeting shRNA (FIG. 6A) or with the bromodomain and extra-terminal (BET) inhibitor, JQ1 (Filippakopoulos et al. 2010) reduced the expression of CD47 and PD-L1 (FIG. 1C). Treatment of MYC TALL cells with the chemotherapeutic drugs prednisone, cytoxan, cisplatin, or vincristine, resulted in tumor cell death. However, CD47 and PD-L1 were either unaffected or showed increased expression (FIG. 7A) and there was no effect on CD3, CD8, CD25, and CD69 expression (FIG. 7B-7E).

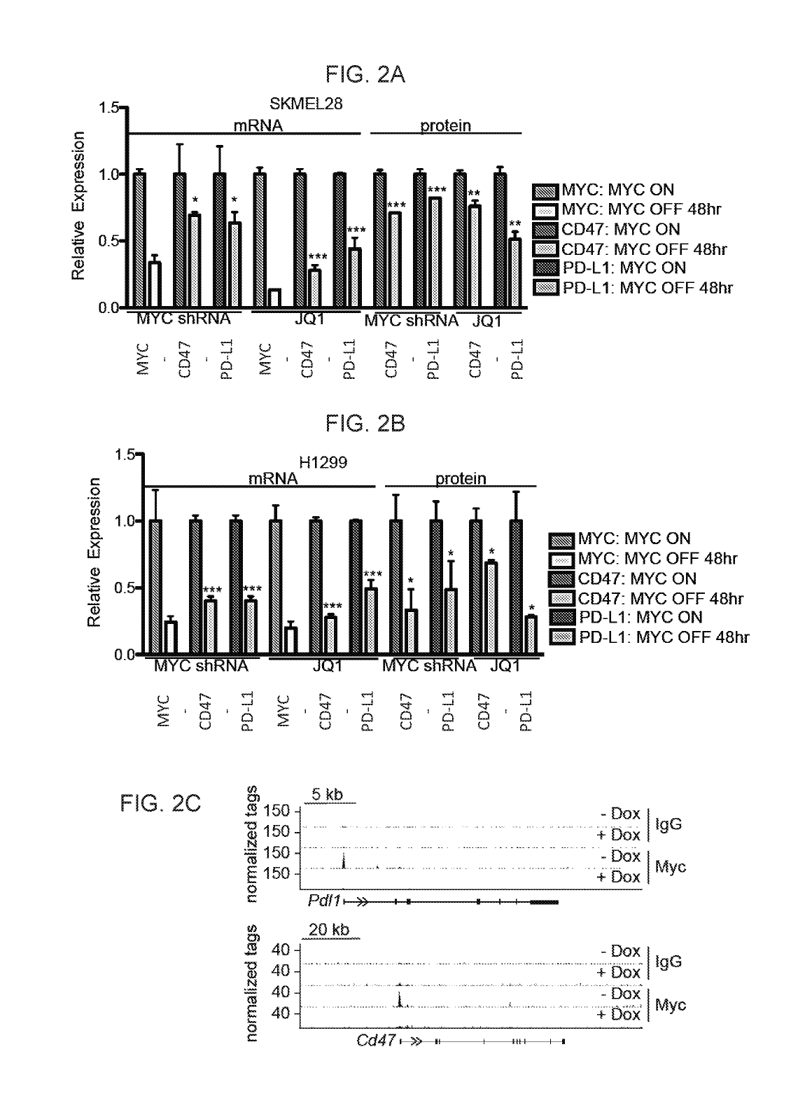

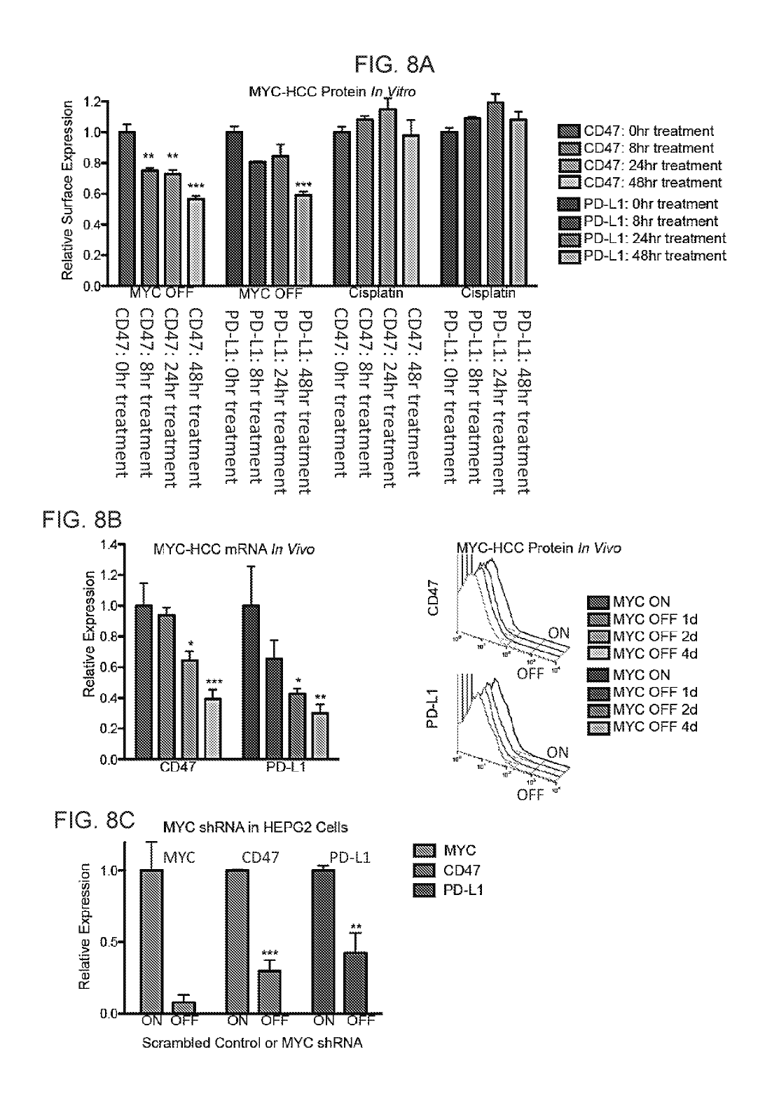

We next investigated the effect of MYC inactivation on CD47 and PD-L1 in mouse and human solid tumors. In a Tet-off transgenic mouse model of hepatocellular carcinoma (HCC) (Shachaf et al. 2004), inhibition of MYC expression resulted in decreased levels of CD47 and PD-L1 protein (FIGS. 8A-8B) and mRNA (FIG. 8B); expression of the two proteins was unaffected by cisplatin treatment (FIG. 8A). In the human HCC cell line HEPG2, shRNA knockdown of MYC caused a reduction in the levels of both CD47 and PD-L1 mRNA (FIG. 8C). We also investigated the relationship between MYC expression and CD47 and PD-L1 expression in the human melanoma cell line SKMEL28 (FIG. 2A) and the human non-small cell lung cancer (NSCLC) cell line H1299 (FIG. 2B), as these cells represent tumor types that are often treated with immune checkpoint inhibitors in the clinic (Moreno et al. 2015). We found that MYC shRNA knockdown and MYC functional suppression by JQ1 reduced the expression of CD47 and PD-L1 mRNA and protein as measured by qPCR and flow cytometry, respectively.

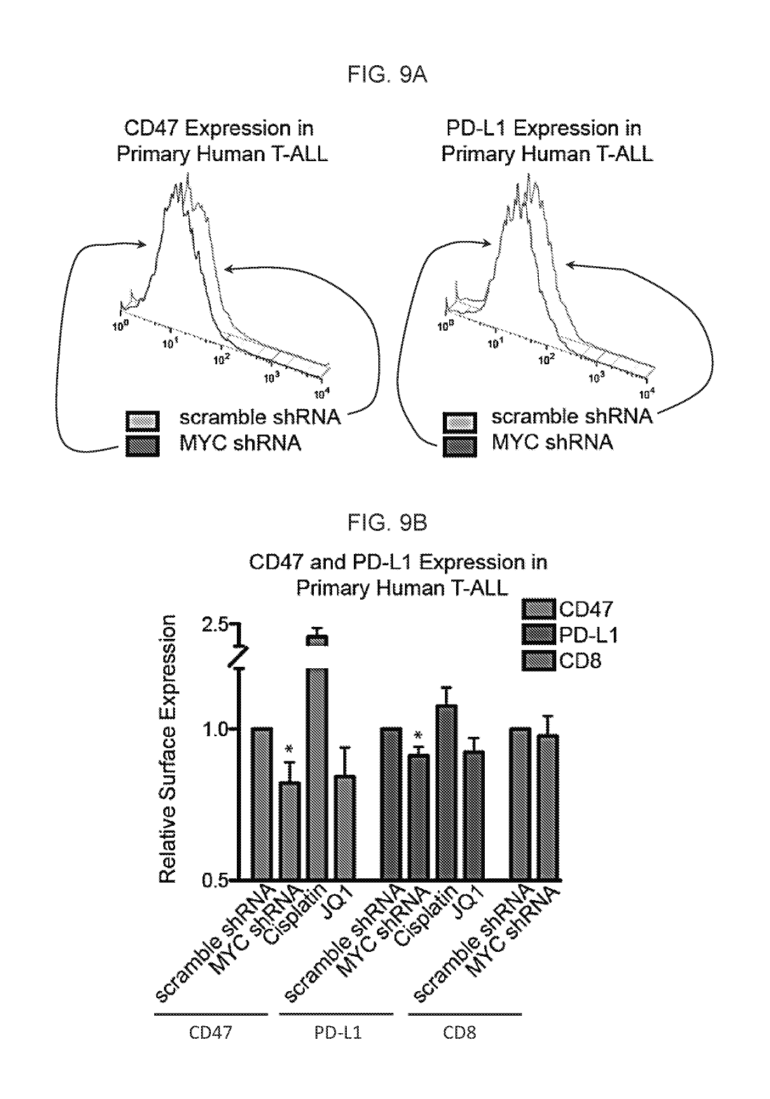

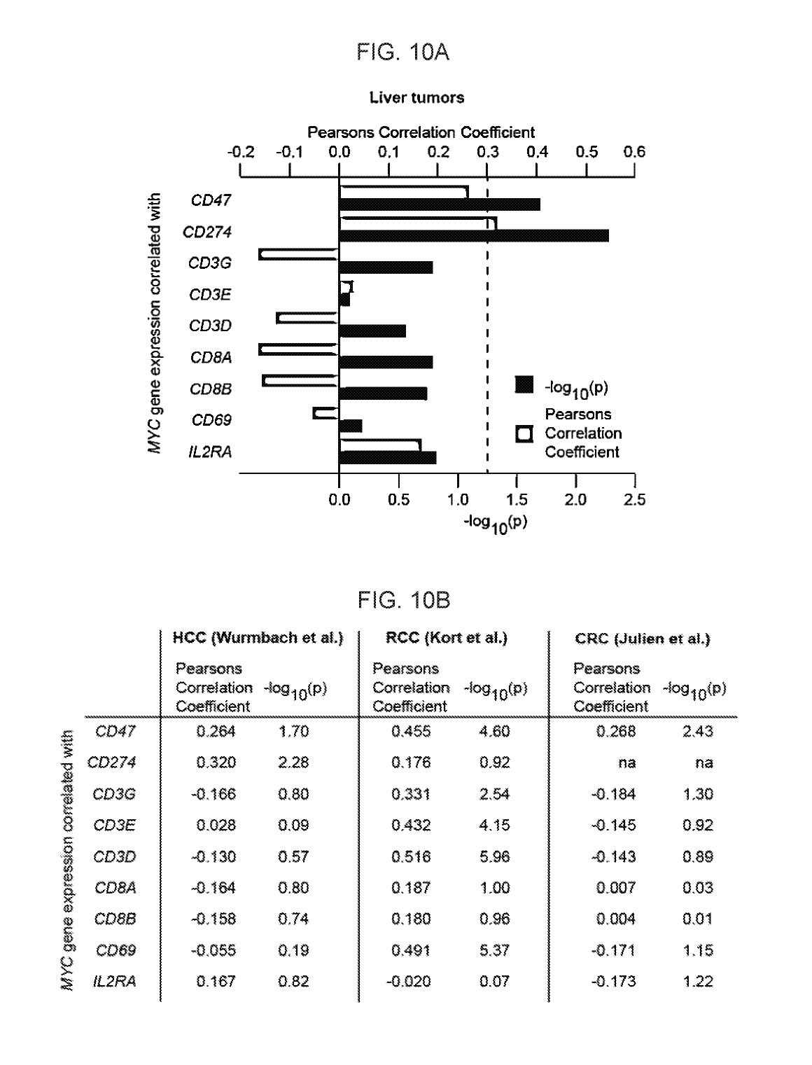

In additional experiments we found that MYC shRNA knockdown (FIG. 6B) or JQ1 treatment of four independent primary human T-ALL samples reduced both CD47 and PD-L1 cell surface expression (FIG. 9A-9B). Cisplatin treatment increased CD47 and PD-L1 expression while CD8 expression was unaffected by the treatments (FIG. 9B). Lastly, we examined publicly available gene expression data derived from human primary tumors. Notably, in human HCC, renal cell carcinoma (RCC), and colorectal carcinoma (CRC), MYC expression significantly correlated with the expression of both CD47 and PD-L1 (FIG. 10A-10B). Thus, MYC regulates CD47 and PD-L1 expression in multiple human tumor types.

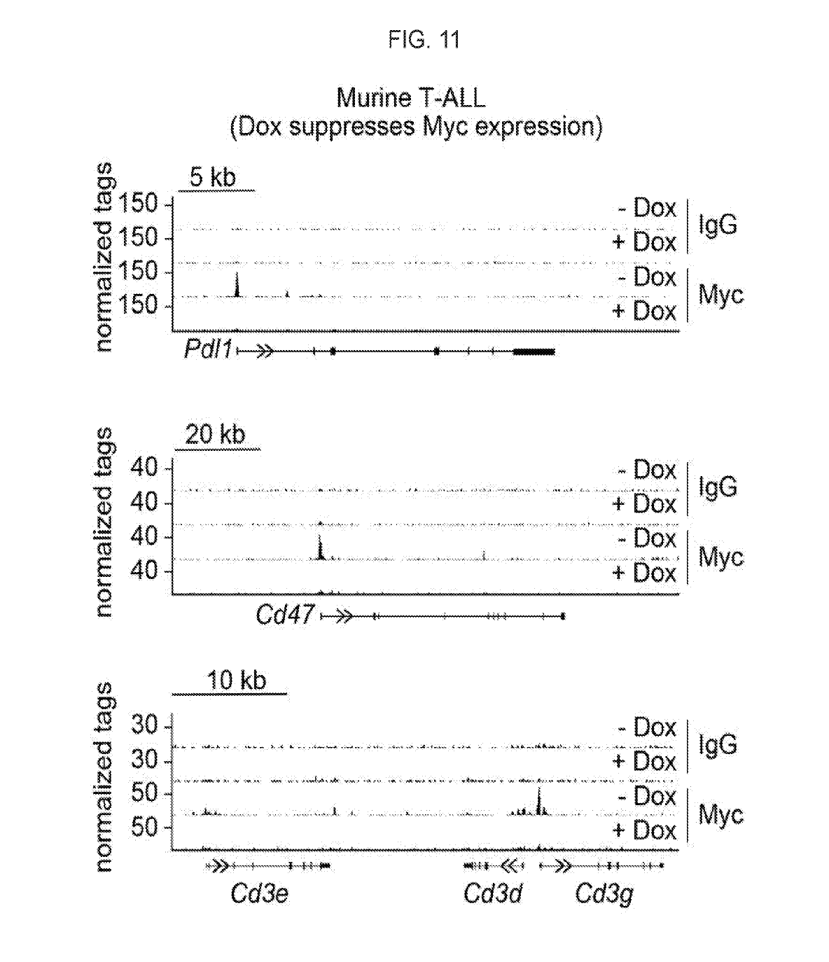

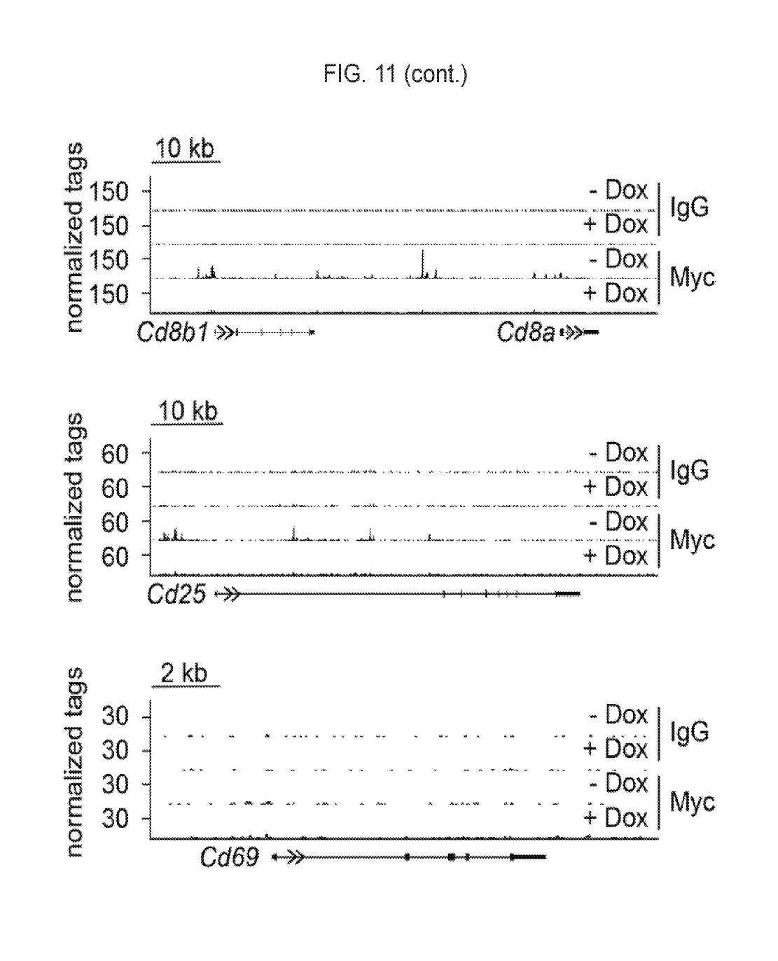

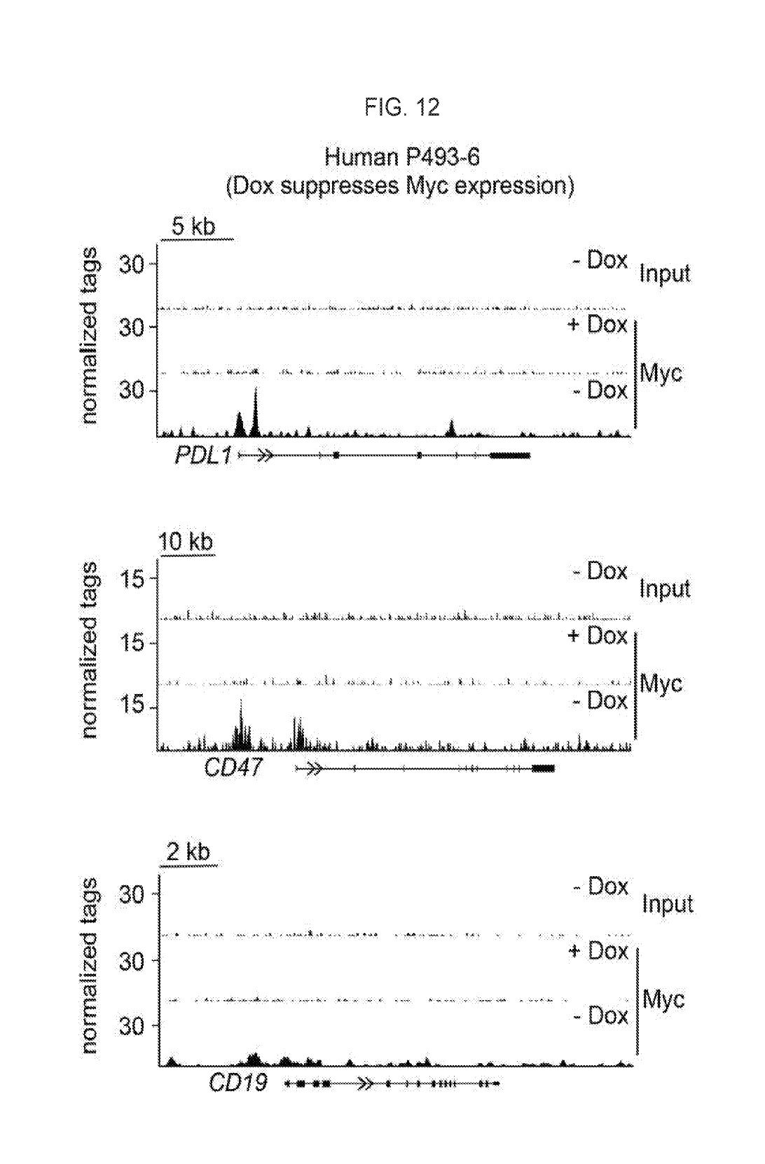

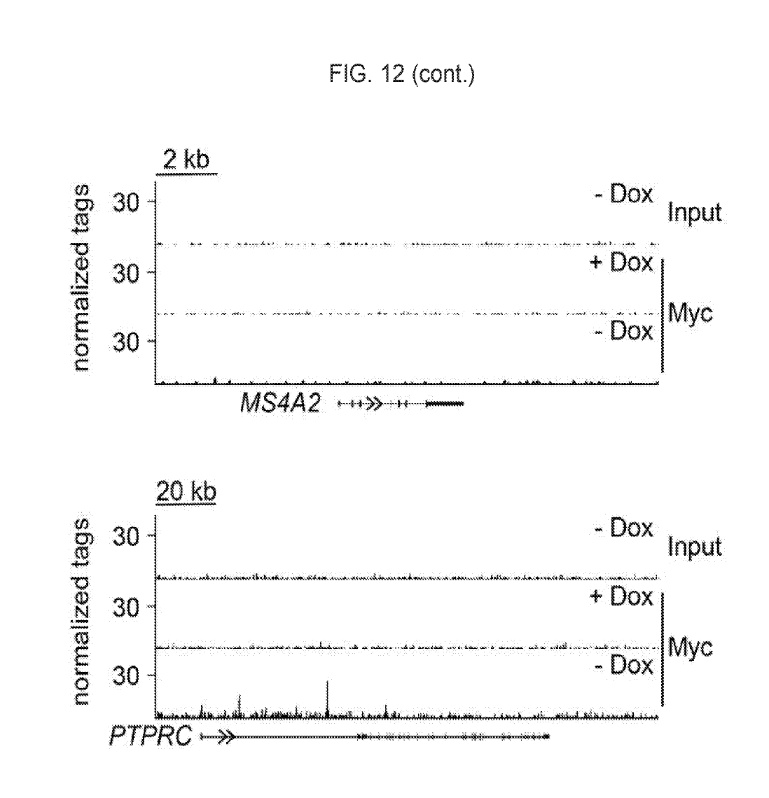

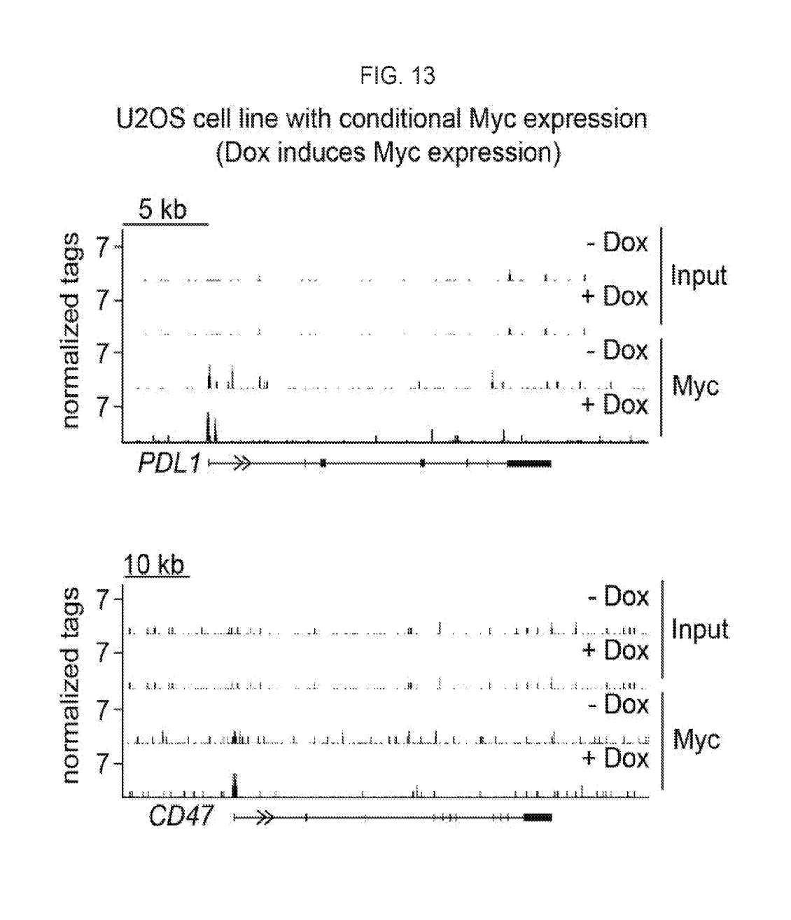

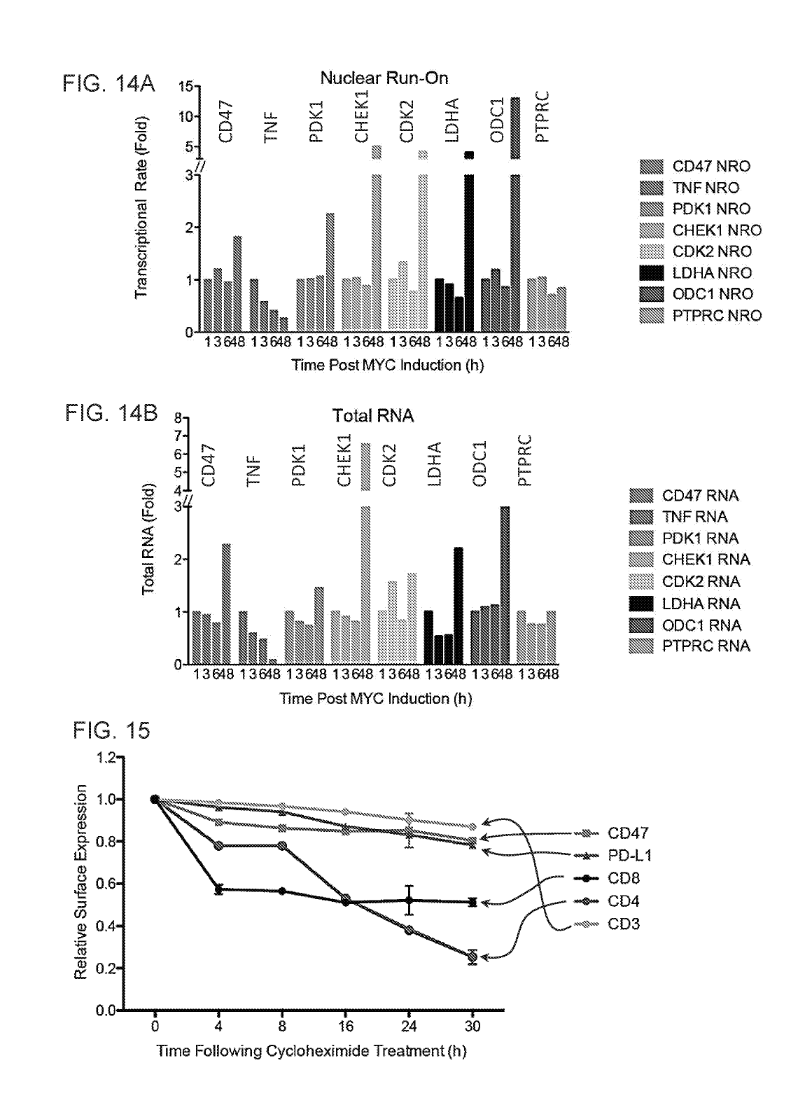

MYC can act as a general transcriptional amplifier (that is, it can generally increase expression of many genes rather than specific target genes), but dosage-dependent specific effects have been reported (Nie et al. 2012, Lin et al. 2012, Walz et al. 2014, Wiese et al. 2013, Choi et al. 2014). We applied ChIP (Chromatin ImmunoPrecipitation)-Seq analysis to mouse MYC T-ALL cells (Walz et al. 2014) and the human B cell line P493-6 (Schuhmacher et al. 1999, Sabo et al. 2014) and found high levels of MYC bound to the promoter regions of the genes coding for CD47 and PD-L1 (FIG. 2C, FIG. 11-12). In contrast, we observed that both MYC T-ALL (FIG. 11) and P493-6 (FIG. 12) cells with high MYC levels had lower, often non-significant binding to the promoters of other cell surface immune molecules such as CD8a and CD25. Oncogenic levels of MYC bound the CD47 and PD-L1 gene promoters in human osteosarcoma U2OS cells, whereas low levels of MYC did not (FIG. 13). In a nuclear run-on assay with P493-6 cells, MYC induced expression of the CD47 gene along with other well-known target genes such as PDK1, CHEK1, CDK2, LDHA, and ODC1 (FIG. 14A-14B).

PD-L1 expression was too low to measure changes in this experiment. Thus, we conclude that MYC binds to the promoters and directly regulates the expression of the CD47 and PD-L1 genes. An alternative but not mutually exclusive possibility is that MYC suppression acutely affects CD47 and PD-L1 surface protein expression by reducing the half-lives of the two proteins. However, we did not observe the increased turnover of CD47 or PD-L1 proteins compared to other immune surface proteins in mouse MYC T-ALL cells when we inhibited protein synthesis by cycloheximide treatment (FIG. 15).

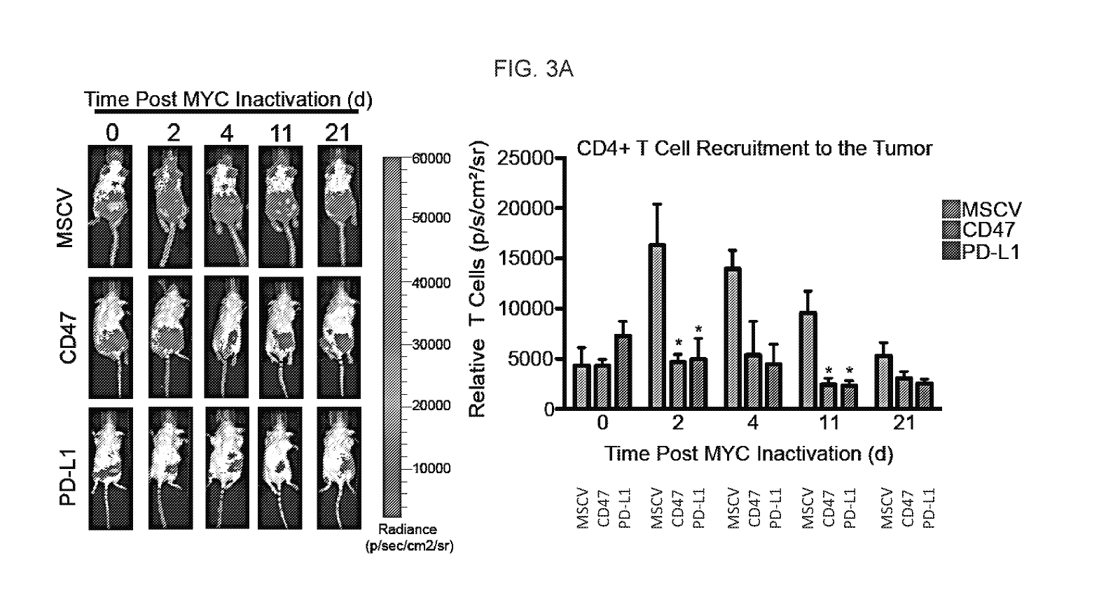

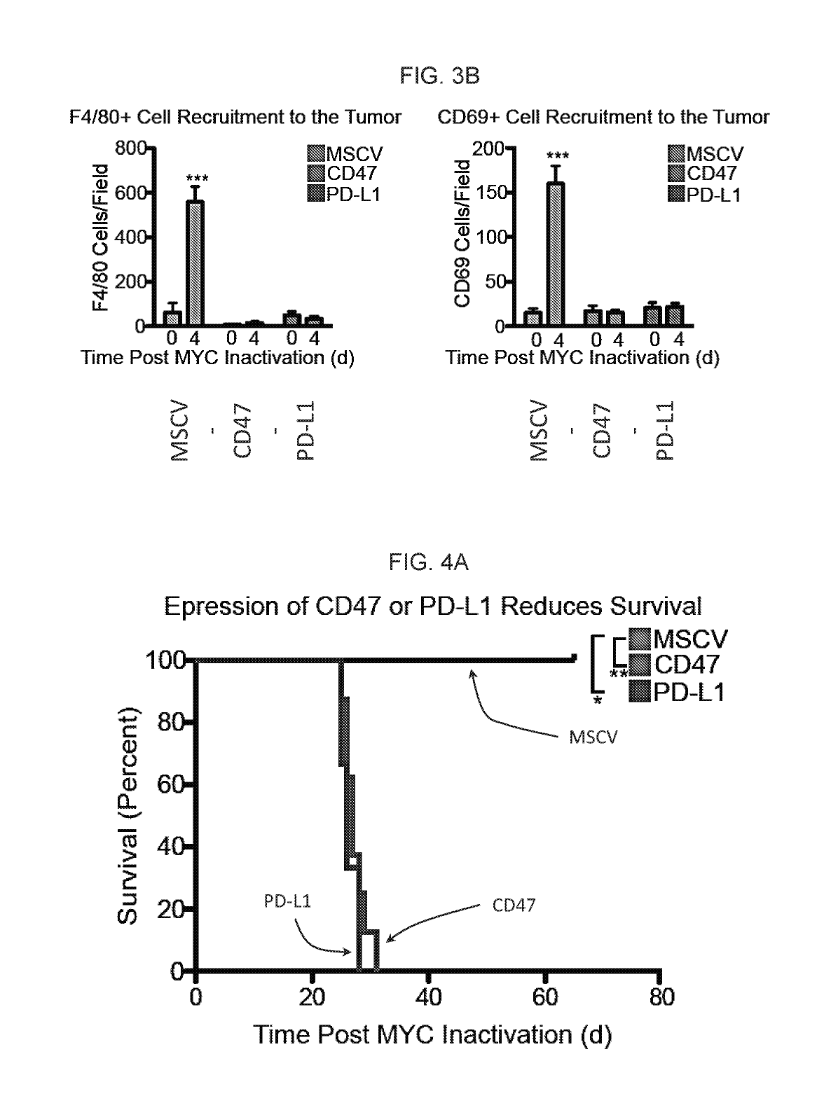

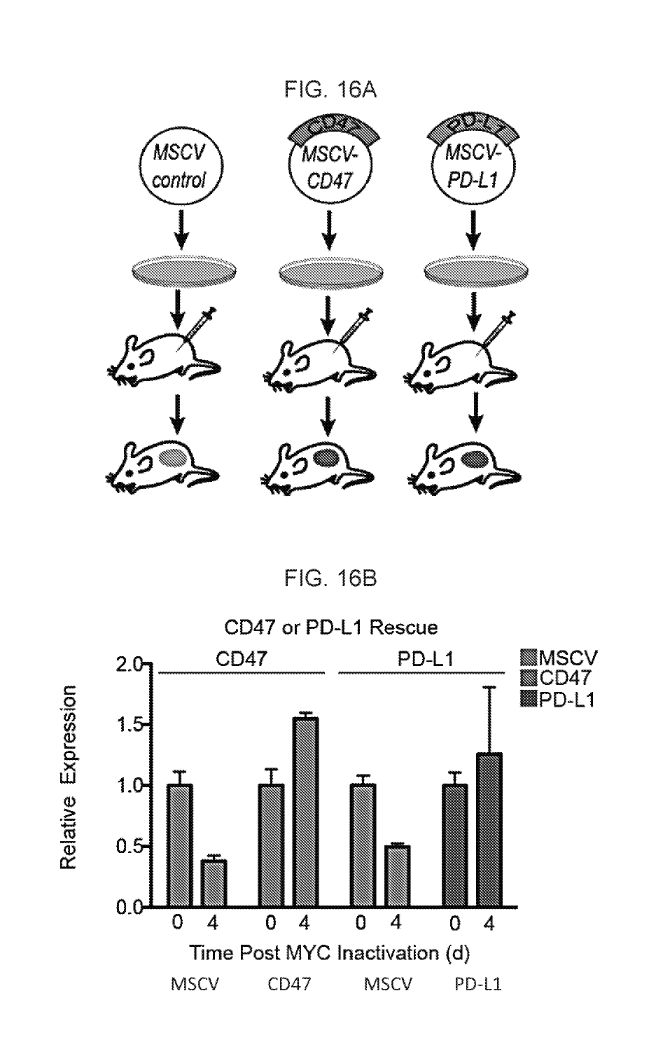

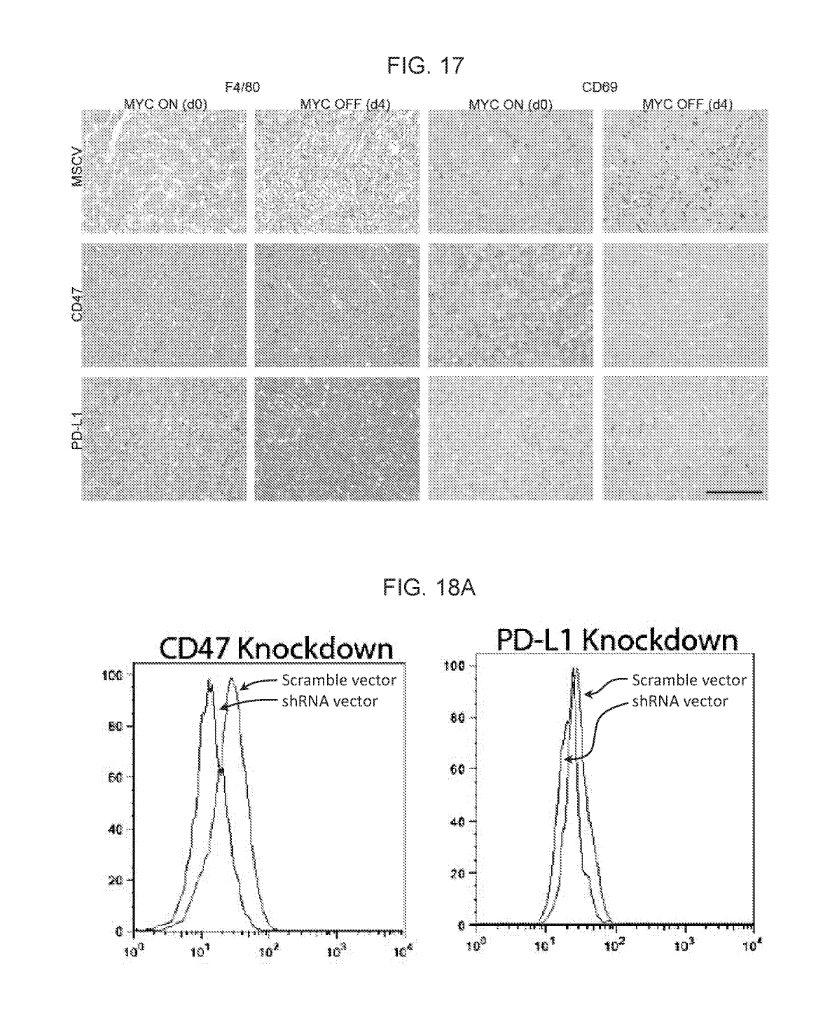

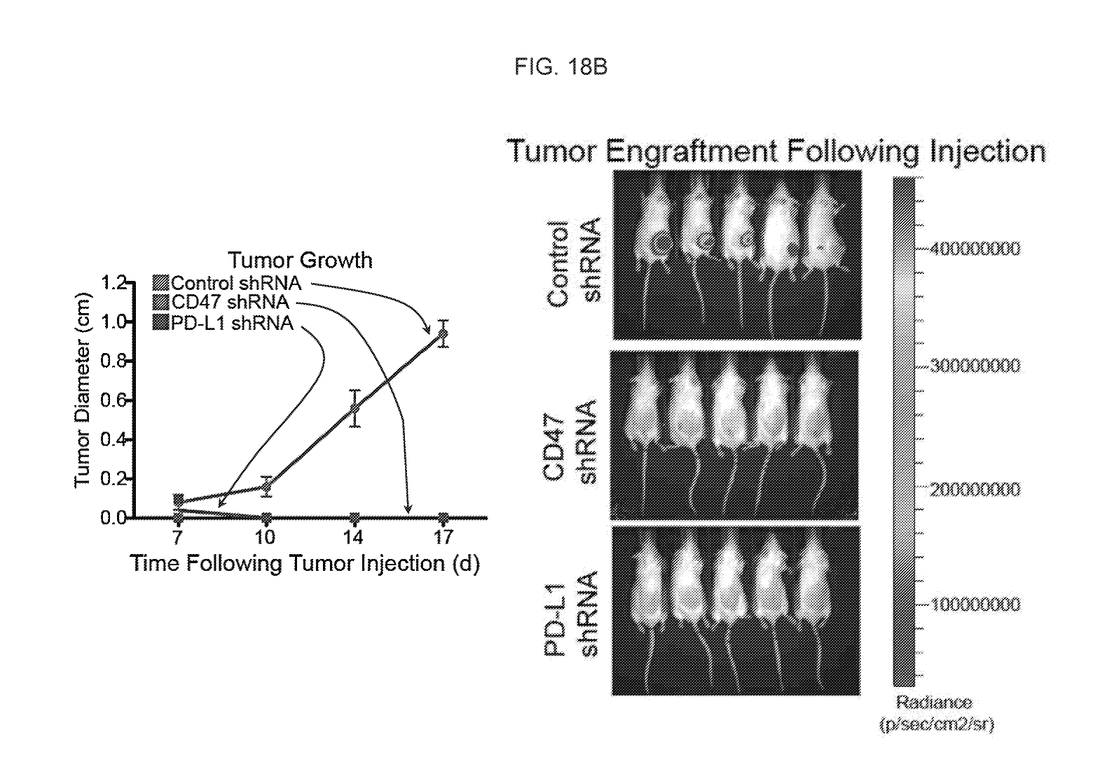

We have shown previously that MYC inactivation in mouse tumor models results in recruitment of immune cells to the tumors (Rakhra et al. 2010). To investigate the role of CD47 and PD-L1 in this process, we engineered MYC T-ALL 4188 cells to constitutively express CD47 or PD-L1 (FIG. 16A). In this overexpression system, CD47 and PD-L1 mRNA levels were unaffected by MYC inactivation (FIG. 16B). The recruitment of luciferase-labeled CD4+ T cells (FIG. 3A), CD69+ activated T cells, and F4/80+ macrophages (FIG. 3B and FIG. 17) following MYC inactivation was suppressed when CD47 and PD-L1 were constitutively expressed by the tumor cells. CD47 or PD-L1 expression prevented the sustained tumor regression that has been observed with MYC inactivation (FIG. 4A) without affecting MYC expression (FIG. 4B). Enforced expression of CD47 or PD-L1 increased minimal residual disease (tumor cells remaining) resulting in tumor recurrence (FIG. 4C-D). Conversely, shRNA knockdown of CD47 or PD-L1 prevented the growth of MYC T-ALL cells in vivo (FIG. 18A-18B).

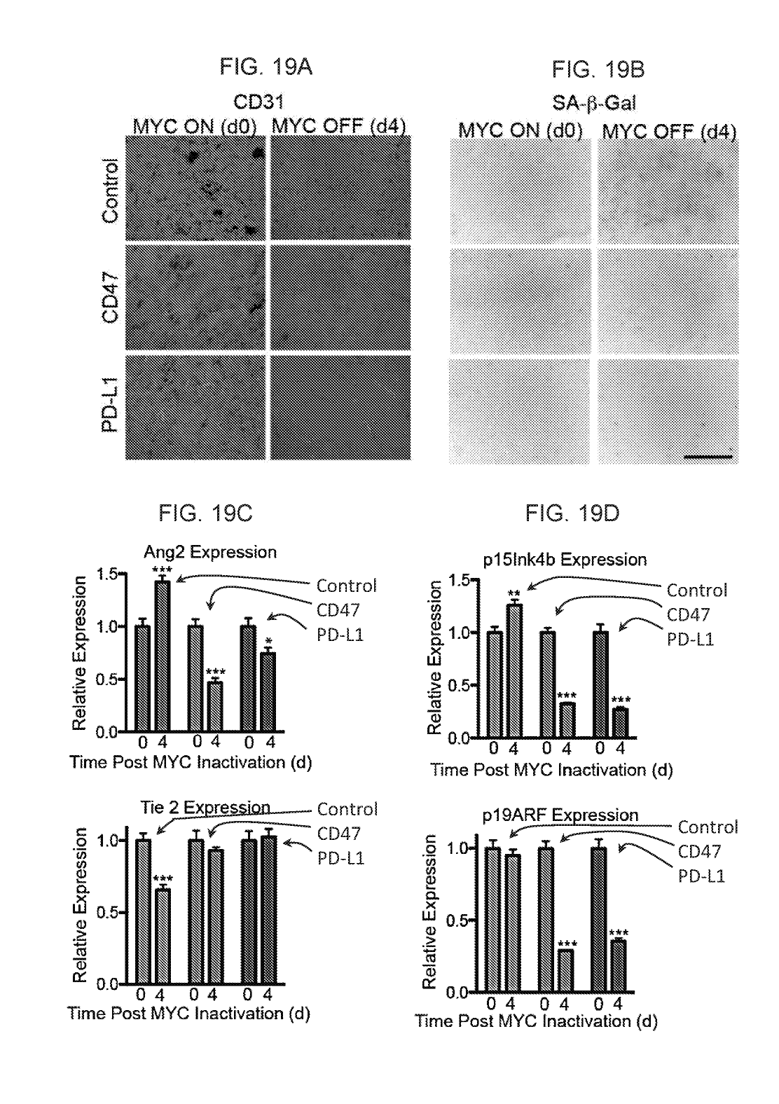

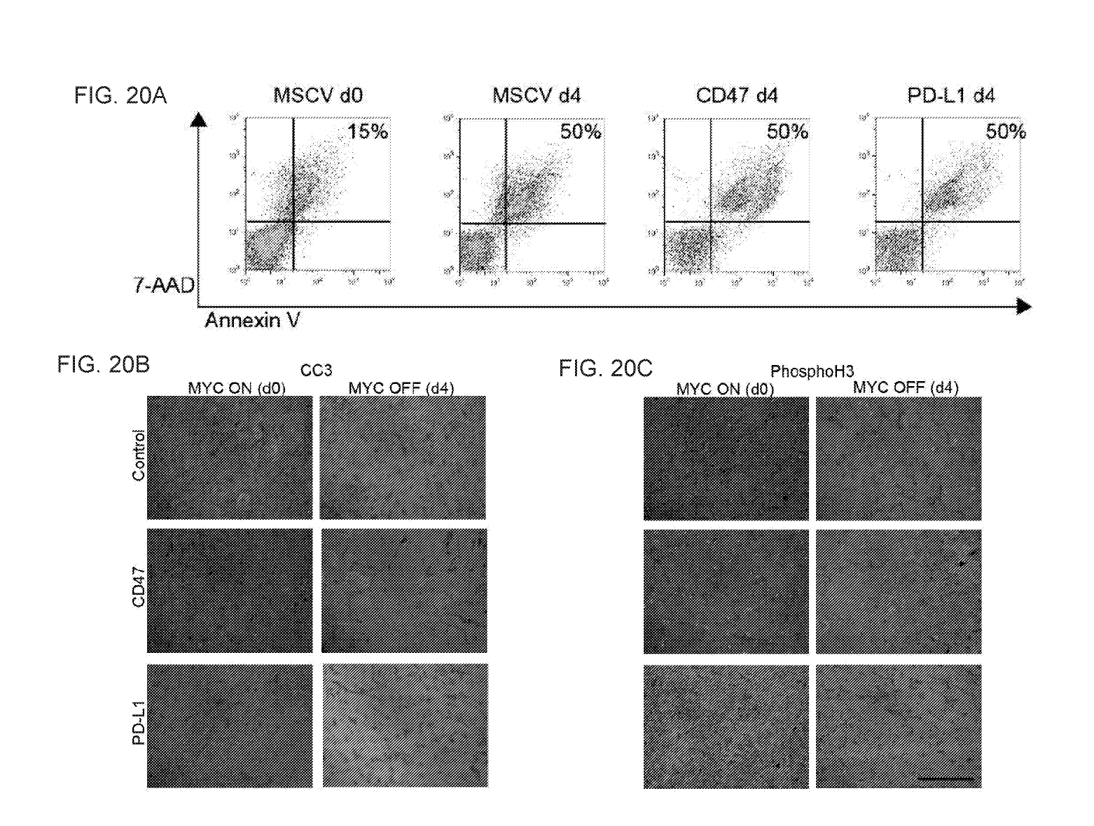

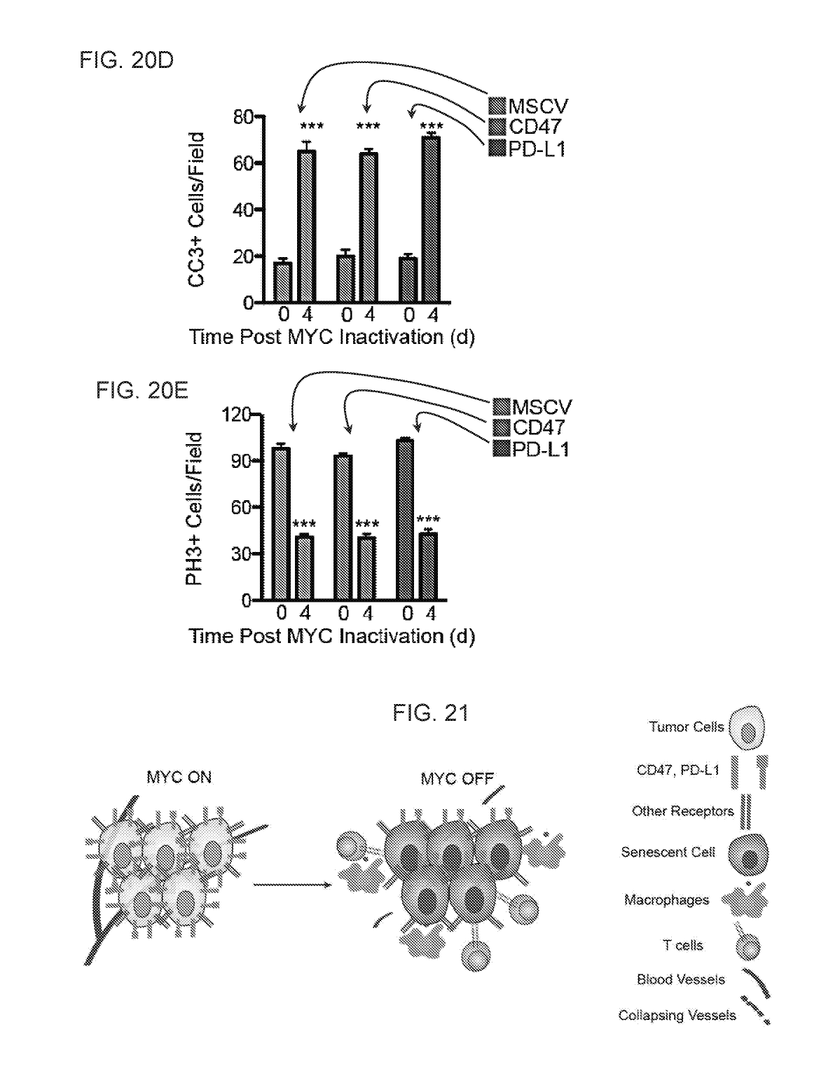

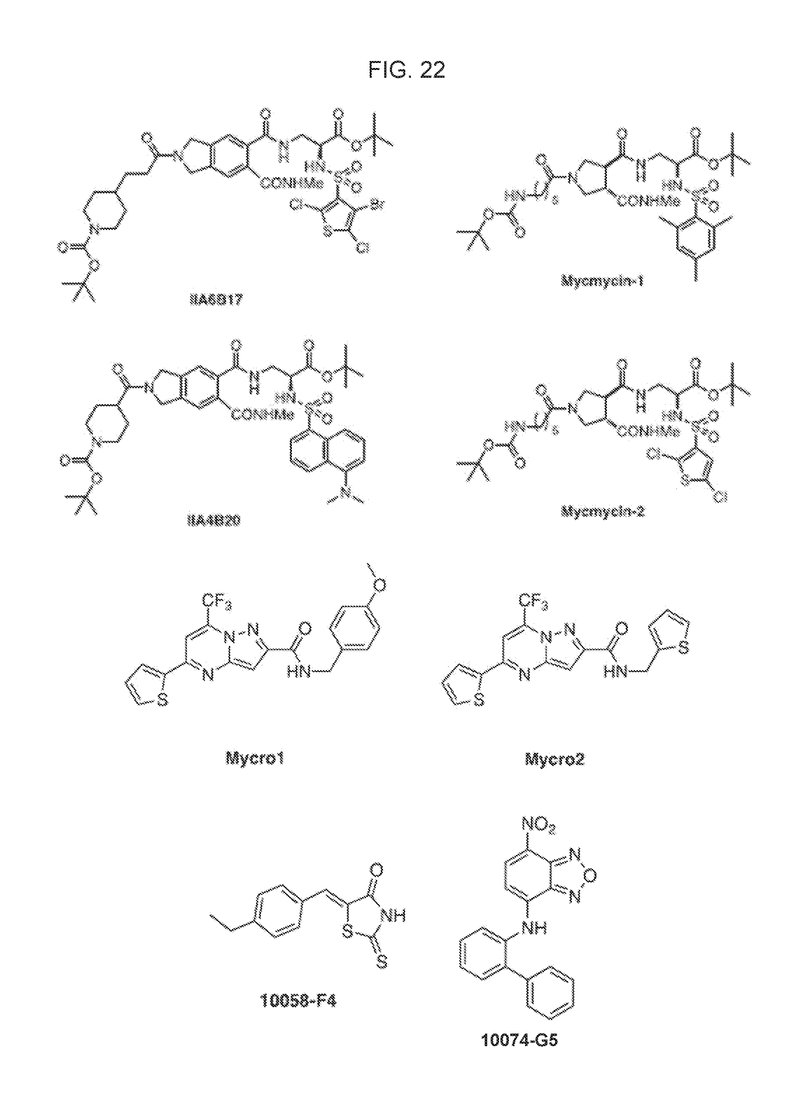

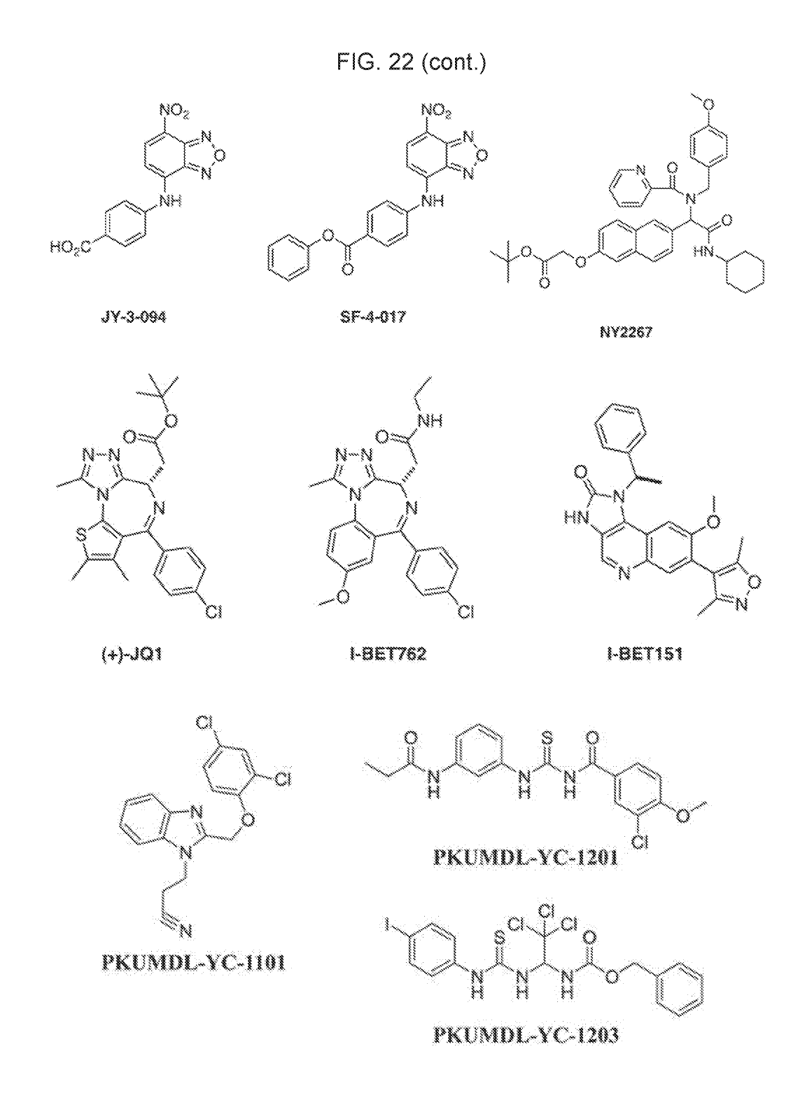

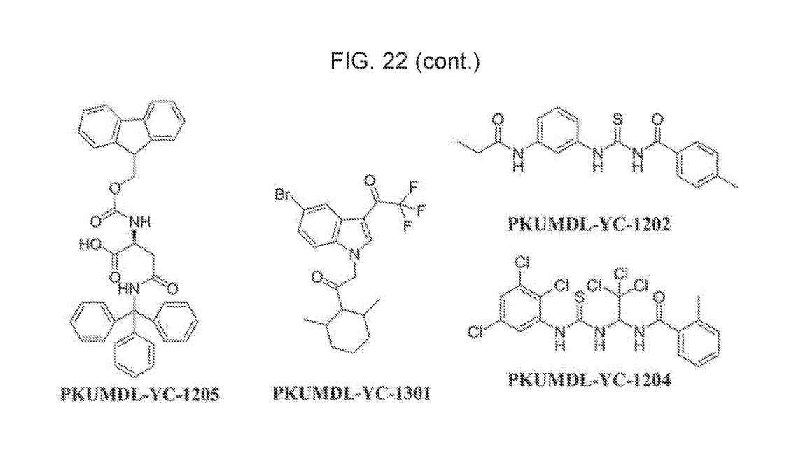

MYC inactivation induces tumor regression through both cell autonomous mechanisms, including proliferative arrest and induction of apoptosis, as well as through host-dependent mechanisms such as inhibition of tumor angiogenesis and induction of tumor cell senescence. We investigated the effect of enforced expression of CD47 or PD-L1 on these mechanisms. We found that CD47 or PD-L1 expression prevented the shutdown of angiogenesis following MYC inactivation, as measured by the presence of CD31+ microvessels (FIGS. 4E, 19A) and expression of Ang2 and Tie2 (FIG. 19C). The induction of tumor cell senescence as measured by β-galactosidase (SA-β-gal) (FIGS. 4F, 19B) and p15lnk4b and p19ARF levels (FIG. 19D) was also affected, but we did not observe an effect on apoptosis or proliferation as evaluated by Annexin-V and 7-AAD (FIG. 20A), cleaved caspase 3 (CC3) (FIGS. 20B, 20D), and Phospho-histone H3 (PH3) (FIGS. 20C, 20E). Therefore, the downregulation of CD47 and PD-L1 appears to be required for the induction of sustained tumor regression, the shutdown of angiogenesis, and senescence induction promoted by MYC inactivation.

We conclude that MYC regulation of CD47 and PD-L1 expression has a direct role in the initiation and maintenance of MYC-driven tumorigenesis (FIG. 4). MYC overexpression may be one general mechanism by which tumor cells upregulate the expression of immune checkpoint regulators, thereby evading immune surveillance. MYC inactivation has been proposed to restore the immune response against tumors (FIG. 21) (Schrieber et al. 2011, Dranoff 2012, Mittal et al. 2014). MYC suppression rapidly resulted in decreased mRNA and protein expression of CD47 and PD-L1, suggesting a transcriptional regulatory mechanism. MYC is a general transcriptional amplifier that can regulate gene expression through a multitude of mechanisms (Nie et al. 2012, Lin et al. 2012, Walz et al. 2014). However, as noted above, MYC also exhibits gene dosage transcriptional effects (Li et al. 2014, Wolf et al. 2015). The relatively high levels of MYC expression that are associated with rapid proliferation and tumorigenesis may induce CD47 and PD-L1 expression.

Because transcription of their genes is regulated by MYC, CD47 and PD-L1 may be expressed at higher levels at steady state than other membrane proteins in tumors. Notably, MYC activation of the CD47 and PD-L1 genes appears to require higher levels of MYC binding to the CD47 and PD-L1 promoters compared with genes involved in normal cell growth; they may, therefore, represent promoters that have been “invaded” by oncogenic MYC levels (Lin et al. 2012, Wolf et al. 2015). Thus, these genes may be particularly sensitive to MYC withdrawal.

MYC activation may influence cancer immunoediting through the suppression of immune surveillance against tumor cells. We propose that during tumor evolution, high MYC expression results in increased expression of CD47 and PD-L1, suppressing both the innate and the adaptive immune response and favoring tumor growth (FIG. 21). Upon MYC inactivation, loss of the “don't find me” and “don't eat me” signals allow for the destruction of residual tumor cells and consequently, sustained tumor regression.

Although the effects of MYC on the expression of CD47 and PD-L1 were modest, the consequences on tumor regression were dramatic, consistent with reports that small influences on immune regulators can have marked effects (Majeti et al. 2005). CD47 and PD-L1 both may also contribute to the tumor microenvironment through influence on T cell activation and angiogenesis (Rakhrs et al. 2010, Giuriato et al. 2006, Topalian et al. 2012, Waclavicek et al. 1997, Avice et al. 2001, Kaur et al. 2013). CD47 is the receptor for Thrombospondin-1, which may regulate cellular programs including angiogenesis, self-renewal and senescence (Rakhra et al. 2010, Giuriato et al. 2006, Kaur et al. 2013). We speculate that therapies suppressing MYC expression and activity may restore an immune response against human cancers. MYC-overexpressing human cancers may be especially vulnerable to an immune checkpoint blockade.

FIG. 1A: Flow cytometry median fluorescence intensity (MFI) was used to determine the relative cell surface expression of CD47, PD-L1, and other immune proteins following MYC inactivation in MYC T-ALL 4188 cells in vitro (n=3). FIG. 1B: Tumors were harvested from primary MYC-driven lymphomas 0 or 4 days following MYC inactivation. mRNA and protein levels were quantified by qPCR and flow cytometry MFI (n=3 tumors per condition). Representative flow cytometry histograms are shown to the right. FIG. 1C: CD47 and PD-L1 protein levels in Jurkat and CCRF-CEM cells were quantified by flow cytometry MFI following MYC inhibition by conditional shRNA knockdown or 10 μM JQ1 treatment (n=3 biological replicates).

FIG. 2A and FIG. 2B: The mRNA and protein levels of MYC, CD47, and PD-L1 in human melanoma SKMEL28 and human NSCLC H1299 cells were determined by qPCR and flow cytometry MFI, respectively, 48 hours after MYC inactivation in vitro. MYC was inactivated by 10 μM JQ1 treatment or MYC shRNA knockdown (n=3 biological and 3 technical replicates for qPCR and 3 biological replicates for flow cytometry). FIG. 2C: ChIP-seq analysis of MYC binding to the promoter sequence of the genes encoding CD47 and PD-L1 in mouse MYC T-ALL cells. IgG was used as a negative control. ChIP-sequencing traces were generated from GSE44672 (Walz et al. 2014). Exons are represented as vertical bars, the untranslated region (UTR) is represented by a black line, and arrows indicate the direction of transcription.

FIG. 3A: Quantification of CD4+ T cells in transplanted control or constitutive CD47 or PD-L1-expressing tumors before 2, 4, 11 or 21 days after MYC inactivation. Control, CD47-expressing, or PD-L1-expressing MYC T-ALL 4188 tumor cells were transplanted into FVB RAG1−/− mice one week after reconstitution with fLuc+ CD4+ T cells. Administration of Dox to inactivate MYC in established tumors is day 0. Left panel: representative bioluminescence images of tumor-bearing RAG1−/− animals. Right panel: average bioluminescence signal of the T cells is shown (n=5 tumors per group). FIG. 3B: Quantification of F4/80+ or CD69+ cells in transplanted control or constitutive CD47 or PD-L1-expressing tumors before or 4 days after MYC inactivation by immunohistochemistry using markers for macrophages (F4/80) and activated T cells (CD69). Tumor cells were transplanted into WT FVB hosts. Administration of Dox to inactivate MYC in established tumors is day 0. The y axis denotes the number of positively staining cells per field. For representative images, see FIG. 17. Data represent mean±SEM derived from measurements of 3 independent tumors and 3 measurements per tumor.

FIG. 4A: Survival after MYC inactivation of syngeneic FVB/N mice that had been transplanted with either MSCV control, CD47-expressing, or PD-L1-expressing fLuc+ MYC T-ALL cells. MYC was inactivated when tumors reached 1.5 cm3 (d0). (n=5 for control, n=10 for CD47, and n=5 for PD-L1). FIG. 4B: MYC expression before (d0) or after MYC inactivation (d4). FIG. 4C: Bioluminescence imaging measurement of tumor burden before and after MYC inactivation in control, CD47-expressing, and PD-L1-expressing tumors. Three representative animals are shown per group. FIG. 4D: Minimal residual disease (remaining tumor cells) after MYC inactivation was measured by bioluminescence imaging. FIG. 4E: Angiogenesis was measured 0 and 4 days after MYC inactivation in control, CD47-expressing, and PD-L1-expressing tumors growing in WT FVB hosts by immunofluorescence for CD31. For representative images, see FIG. 19A-19D. FIG. 4F: Control, CD47-expressing, and PD-L1-expressing tumors were analyzed by immunostaining for senescence associated β-galactosidease (SA-β-gal) in tumors described in FIG. 4E. The y axis denotes the number of positively staining microvessels FIG. 4E or cells FIG. 4F per field. For representative images, see FIG. 19B. Data represent mean±SEM derived from measurements of 3 independent tumors and 3 measurements per tumor.

FIG. 5A: Schematic showing two key immune regulatory proteins expressed on the cell surface of tumor cells, CD47 and PD-L1, and the immune cells that recognize them. FIG. 5B: Protein expression of CD47 and PD-L1 in murine primary MYC T-All before and after MYC inactivation. Tumors were harvested from primary MYC T-ALL transgenic animals 0 (MYC ON) or 4 days (MYC OFF) following MYC inactivation with Dox. Immunofluorescence staining of CD47 and PD-L1 (labeled in dark pink, DAPI counterstain) was performed. Images are representative of 3 different tumors per condition. Scale bar=50 μm.

FIG. 6A-6B: MYC mRNA levels before and after treatment with shRNA against MYC or treatment with JQ1. MYC mRNA was measured by qPCR in human T-ALL cell lines (A, n=3) and primary human T-ALL (B, n=4) cells following MYC inhibition by shRNA or JQ1 treatment after the indicated number of hours (0-48 h for human T-ALL cell lines, 48 h for primary T-ALL).

FIG. 7A: CD47 and PD-L1 protein levels in MYC T-ALL 4188 cells treated with prednisone, cytoxan, cisplatin, or vincristine (10 μM each) were quantified by flow cytometry MFI. Means±SEM are shown, n=3. FIG. 7B-7E: CD3, CD8, CD25, and CD69 protein levels in MYC T-ALL 4188 cells treated as in FIG. 7A were quantified by flow cytometry. Means±SEM are shown (n=3).

FIG. 8A: CD47 and PD-L1 protein levels in cells derived from mouse MYC-driven HCC, following MYC inactivation or chemotherapy treatment, were quantified by flow cytometry MFI (n=3). FIG. 8B: CD47 and PD-L1 mRNA levels were quantified by qPCR and protein levels were quantified by flow cytometry in primary tumors from mouse MYC-driven HCC, following MYC inactivation (n=3 tumors per condition). (FIG. 8C: MYC, CD47, and PD-L1 mRNA levels in human HEPG2 HCC cells were quantified by qPCR 48 h after MYC shRNA knockdown. n=3. Means±SEM are shown.

FIG. 9A: CD47 and PD-L1 protein levels in primary human T-ALL tumor samples were quantified by flow cytometry, following 48 h treatment with control scramble shRNA or MYC-targeting shRNA. FIG. 9B: CD47, PD-L1, or CD8 protein levels in primary human T-ALL tumor samples were quantified by flow cytometry MFI, following 48 h treatment with control scramble shRNA or MYC-targeting shRNA, or treatment with 10 μM JQ1 or 10 μM cisplatin (n=4 distinct patient samples). Means±SEM are shown.

FIG. 10A: Correlation of MYC gene expression with the expression of several immune-related surface receptors in a dataset of human HCCs (n=75, (Wurmbach et al. 2007)). The dashed line indicates P=0.05. FIG. 10B: Three datasets of human HCC (n=75, (Wurmbach et al. 2007)), RCC (n=79, (Kort et al. 2008)) and CRC (n=115, (Julien et al. 2012)) were analyzed for correlation of MYC expression with immune-related cell surface molecules. Pearsons correlation coefficients were determined using one representative probe for each gene (“na”: no probe was detected for that gene) and P-values are given as negative common logarithm to the base 10.

FIG. 11: MYC DNA binding in conditional transgenic mouse MYC T-ALL cells. ChIP-sequencing traces were generated from GSE44672 (Walz et al. 2014). IgG was used as a negative control. Exons are represented as vertical bars, the UTR is represented by a black line, and arrows indicate the direction of transcription.

FIG. 12: MYC DNA binding in human P493-6 Burkitt lymphoma-like cells. Binding profiles were generated from GSE51004 (Sabo et al. 2014). Exons are represented as vertical bars, the UTR is represented by a black line, and arrows indicate the direction of transcription.

FIG. 13. MYC DNA binding in conditional MYC-driven U2OS cells. ChIP-sequencing traces were generated from GSE44672 (Walz et al. 2014). Exons are represented as vertical bars, the UTR is represented by a black line, and arrows indicate the direction of transcription.

FIG. 14A-14B: Measurement of RNA by nuclear run-on analysis after MYC induction. Nuclear run-on analysis of CD47, TNF, PDK1, CHEK1, CDK2, LDHA, ODC1, and PTPRC gene transcription following MYC activation in P493-6 cells.

FIG. 15: Relative protein stability of immune surface receptors following cycloheximide treatment. MYC T-ALL 4188 cells were treated with 20 μg/mL cycloheximide and the relative surface expression of immune surface molecules was quantified by flow cytometry at the indicated time points (n=3). Means±SEM are shown.

FIG. 16A: General strategy for maintaining expression of CD47 or PD-L1 after MYC inactivation. Retroviral mediated expression of CD47 and PD-L1 in MYC T-ALL 4188 cells. Empty MSCV retroviral vector (control) or retroviral vector containing either CD47 or PD-L1 cDNA was used to infect MYC T-ALL 4188 cells to maintain expression of CD47 or PD-L1 even upon MYC inactivation. FIG. 16B: mRNA levels of CD47 and PD-L1 in transplanted control or constitutive CD47/PD-L1-expressing tumors at 0 and 4 days following MYC inactivation in WT mice (FVB strain) (n=3 tumors per condition). Means±SEM are shown.

FIG. 17: Sustained expression of CD47 or PD-L1 inhibits the recruitment of immune cells following MYC inactivation. Immunohistochemistry of macrophages (F4/80) and activated lymphocytes (CD69) in control MSCV, CD47-expressing, and PD-L1-expressing tumors following MYC inactivation in WT FVB hosts. Images are representative of 3 distinct tumors and 3 images per tumor. Scale bar: 100 μm.

FIG. 18A: MYC T-ALL 4188 cells were infected with lentivirus carrying the control scrambled shRNA or shRNA targeting CD47 or PD-L1, and CD47 (left) or PD-L1 (right) surface protein expression was detected. FIG. 18B: Representative mice with bioluminescence indicating tumor growth are shown 17 d after the injection of tumor cells expressing CD47- or PD-L1-targeting shRNA (right). Tumor diameters were measured by calipers and plotted as the mean±SEM (left). n=5 mice per condition.

FIG. 19A: Immunofluorescence of CD31 (detecting angiogenesis) in control, CD47-expressing, or PD-L1-expressing tumors (generated as described in FIG. 16A) 0 or 4 d following MYC inactivation. Quantification is shown in FIG. 4E. FIG. 19B: SA-β-gal (induction of senescence) staining of tumors described in (FIG. 19A). Quantification is shown in FIG. 4F. Scale bar: 100 μm. Images are representative of 3 distinct tumors and 3 images per tumor. Relative expression as measured by qPCR of (FIG. 19C) Ang2, Tie2, (FIG. 19D) p15lnk4b, and p19ARF in control, CD47-expressing, and PD-L1-expressing tumors before (d0) and after MYC inactivation (d4). Graphs are presented as the mean±SEM (n=3 distinct tumors per condition).

FIG. 20A: Annexin V and 7-AAD were labeled in MYC T-ALL 4188 cells expressing control pMSCV, CD47, or PD-L1 following MYC inactivation in vitro and detected by flow cytometry (n=3). Cells in the upper right quadrant represent the percentage of apoptotic cells. FIG. 20B: CC3 immunofluorescence detecting apoptosis (dark pink stain, DAPI counterstain) of tumors as indicated in FIG. 4. Scale bar=100 μm. Images are representative of 3 distinct tumors and 3 images per tumor. (FIG. 20C: PH3 immunofluorescence detecting cells in metaphase (dark pink stain, DAPI counterstain) of tumors as indicated in FIG. 4. (FIG. 20D-20E: Quantification of CC3 (FIG. 20D) and PH3 (FIG. 20E) immunofluorescence. Graphs are presented as the mean±SEM (n=3 distinct tumors per condition). The y axis denotes the number of positively staining cells per field.

FIG. 21. MYC regulates the immune response against tumor cells via CD47 and PD-L1. Model demonstrating the regulation of immunological checkpoints in MYC-driven tumors. Following MYC inactivation, the expression of CD47 and PD-L1 is reduced, leading to an immune response and the subsequent induction of senescence and shutdown of angiogenesis.

TABLE 1
Primer sequences used for qPCR analysis
of mRNA expression.

| | | SEQ |
|---|---|---|
| | | ID |
| Primer | Sequence | NO: |
| Transgenic MYC Forward | CTGCGACGAGGAGGAGAACT | 21 |
| Transgenic MYC Reverse | GGCAGCAGCTCGAATTTCTT | 22 |
| Ang2 Forward | AGCAGATTTTGGATCAGACCAG | 23 |
| Ang2 Reverse | GCTCCTTCATGGACTGTAGCTG | 24 |
| Tie2 Forward | CGGCTTAGTTCTCTGTGGAGTC | 25 |
| Tie2 Reverse | GGCATCAGACACAAGAGGTAGG | 26 |
| p151nk4b Forward | TCTTGGATCTCCACAAGC | 27 |
| p151nk4b Reverse | CTCCAGGTTTCCCATTTAC | 28 |
| p19ARF Forward | GTCGCAGGTTCTTGGTCACT | 29 |
| p19ARF Reverse | ATCGCACGAACTTCACCAA | 30 |
| C047 Forward | TGCGGTTCAGCTCAACTACTG | 31 |
| C047 Reverse | GCTTTGCGCCTCCACATTAC | 32 |
| PO-L 1 Forward | GCTCCAAAGGACTTGTACGTG | 33 |
| PO-L 1 Reverse | TGATCTGAAGGGCAGCATTTC | 34 |
| UBC Forward | AGCCCAGTGTTACCACCAAG | 35 |
| UBC Reverse | ACCCAAGAACAAGCACAAGG | 36 |
| hC047 Forward | GGCAATGACGAAGGAGGTTA | 37 |
| hC047 Reverse | ATCCGGTGGTATGGATGAGA | 38 |
| hPD-L1 Forward | GGCATTTGCTGAACGCAT | 39 |
| hPD-L1 Reverse | CAATTAGTGCAGCCAGGT | 40 |

Notwithstanding the appended clauses, the disclosure is also defined by the following clauses:

1. A method of identifying whether a subject having a cancer will be responsive to an immune checkpoint inhibitor, the method comprising:

(a) determining whether a cellular sample from the cancer overexpresses a MYC oncogene; and

(b) identifying the subject as (i) responsive to the immune checkpoint inhibitor when the determining indicates that the cancer overexpresses the MYC oncogene or (ii) as nonresponsive to the immune checkpoint inhibitor when the determining indicates that the cancer does not overexpress the MYC oncogene.

2. The method according to Clause 1, wherein the immune checkpoint inhibitor is a CD47 inhibitor.
3. The method according to Clause 1, wherein the immune checkpoint inhibitor is a PD-L1 inhibitor.
4. The method according to any of Clauses 1 to 3, wherein the method further comprises measuring the level of expression of the MYC oncogene in the cellular sample.
5. The method according to Clause 4, wherein the level of expression comprises an mRNA expression level.
6. The method according to Clauses 4 or 5, wherein the level of expression comprises a protein expression level.
7. The method according to any of Clauses 1 to 6, wherein the cancer is selected from the group consisting of: a lymphoma/leukemia, a carcinoma and a melanoma.
8. The method according to Clause 7, wherein the lymphoma/leukemia is acute lymphoblastic leukemia.
9. The method according to Clause 7, wherein the carcinoma is selected from the group consisting of: a hepatocellular carcinoma, a non-small cell lung carcinoma, a renal cell carcinoma and a colorectal carcinoma.
10. A method of treating a subject for a cancer, the method comprising:

(a) identifying whether the cancer will be responsive to an immune checkpoint inhibitor according to any of Clauses 1 to 9; and

(b) administering an effective amount of an agent to the subject to treat the cancer, wherein when the cancer is identified as responsive the agent is the immune checkpoint inhibitor.

11. A method of treating a subject for an immune-evasive cancer, the method comprising:

(a) determining whether a cellular sample of the immune-evasive cancer obtained from the subject overexpresses CD47, PD-L1 or both; and

(b) administering an effective amount of an agent to the subject to treat the immune-evasive cancer, wherein when the cellular sample is determined to overexpress CD47, PD-L1 or both the agent is a MYC inhibitor.

12. The method according to Clause 11, wherein the immune-evasive cancer is a recurrent cancer.
13. The method according to Clauses 11 or 12, wherein the immune-evasive cancer is a cancer that was nonresponsive to a prior cancer therapy.
14. The method according to Clause 13, wherein the prior cancer therapy comprised administering to the subject a chemotherapeutic agent selected from the group consisting of: prednisone, cytoxan, cisplatin, and vincristine.
15. The method according to any of Clauses 11 to 14, wherein the MYC inhibitor is a bromodomain and extra-terminal (BET) inhibitor.
16. The method according to Clause 15, wherein the BET inhibitor is JQ1 or an analog thereof.
17. The method according to any of Clauses 11 to 16, wherein the method further comprises administering to the subject a chemotherapeutic agent.
18. The method according to Clause 17, wherein the chemotherapeutic agent is selected from the group consisting of: prednisone, cytoxan, cisplatin, and vincristine.
19. The method according to any of Clauses 11 to 18, wherein the method further comprises measuring a level of expression of CD47, PD-L1 or both in the cellular sample.
20. The method according to Clause 19, wherein the level of expression comprises an mRNA expression level.
21. The method according to Clauses 19 or 20, wherein the level of expression comprises a protein expression level.
22. The method according to any of Clauses 11 to 21, wherein the immune-evasive cancer is selected from the group consisting of: a lymphoma/leukemia, a carcinoma and a melanoma.
23. The method according to Clause 22, wherein the lymphoma/leukemia is acute lymphoblastic leukemia.
24. The method according to Clause 22, wherein the carcinoma is selected from the group consisting of: a hepatocellular carcinoma, a non-small cell lung carcinoma, a renal cell carcinoma and a colorectal carcinoma.
25. A method of identifying a therapeutic agent that is effective during MYC-regulated immune evasion of a cancer cell, the method comprising:

(a) contacting (i) a first tumor cell line conditionally expressing a MYC oncogene and (ii) a second tumor cell line conditionally expressing a MYC oncogene and a MYC-independent constitutively expressed immune checkpoint protein with a candidate therapeutic agent;

(b) determining the effect of the contacting on a behavior of the contacted conditional tumor cell lines;

(c) identifying the candidate agent as a therapeutic agent that is effective during MYC-regulated immune evasion of a cancer cell if the determined behavior of the first conditional tumor cell line (i) is reduced as compared to a non-contacted control and the determined behavior of the second conditional tumor cell line (ii) is not reduced as compared to a non-contacted control.

26. The method according to Clause 25, wherein the contacting is performed in vitro.
27. The method according to Clause 26, wherein the in vitro contacting comprises three-dimensional cell culture.
28. The method according to Clause 25, wherein the contacting is performed in vivo.
29. The method according to Clause 28, wherein the contacting is performed in a non-human mammal.
30. The method according to Clause 29, wherein the non-human mammal is a rodent.
31. The method according to Clause 30, wherein the rodent is a mouse.
32. The method according to any of Clauses 25 to 31, wherein the MYC-independent constitutively expressed immune checkpoint protein is CD47.
33. The method according to any of Clauses 25 to 32, wherein the MYC-independent constitutively expressed immune checkpoint protein is PD-L1.
34. The method according to any of Clauses 25 to 33, wherein the second tumor cell line constitutively expresses both CD47 and PD-L1.
35. The method according to any of Clauses 25 to 34, wherein the behavior comprises cell line expansion.
36. The method according to Clause 35, wherein the method comprises quantifying cell line expansion.
37. The method according to any of Clauses 25 to 36, wherein the behavior comprises cell line survival.
38. The method according to Clause 37, wherein the method comprises quantifying cell line survival.
39. The method according to any of Clauses 25 to 38, wherein the therapeutic agent that overcomes MYC-regulated immune evasion inhibits MYC induced expression of one or more immune checkpoint genes.
40. The method according to Clause 39, wherein the one or more immune checkpoint genes is CD47, PD-L1 or both.
41. The method according to any of Clauses 25 to 40, wherein the method further comprises quantifying the expression of the one or more immune checkpoint genes.
42. The method according to Clause 41, wherein the quantifying comprises mRNA expression analysis of the one or more immune checkpoint genes.
43. The method according to Clauses 41 or 42, wherein the quantifying comprises protein expression analysis of the one or more immune checkpoint genes.
44. The method according to any of Clauses 25 to 43, wherein the method further comprises providing the first and second tumor cell lines.
45. The method according to any of Clauses 25 to 44, wherein the conditional MYC expression of the first and second cell lines is controlled by a tetracycline-based system.
46. The method according to any of Clauses 25 to 44, wherein the conditional MYC expression of the first and second cell lines is controlled by a tamoxifen-based system.
47. The method according to any of Clauses 25 to 46, wherein the first and second tumor cell lines are derived from a cancer selected from the group consisting of: lymphoma/leukemia, carcinoma and melanoma.
48. The method according to Clause 47, wherein the lymphoma/leukemia is acute lymphoblastic leukemia.
49. The method according to Clause 47, wherein the carcinoma is selected from the group consisting of: hepatocellular carcinoma, non-small cell lung carcinoma, renal cell carcinoma and colorectal carcinoma.
50. A cancer cell line comprising:

(a) an inducible MYC locus comprising a MYC encoding nucleic acid sequence operably linked to a transcriptional response element; and

(b) a MYC-independent constitutively active locus comprising an immune checkpoint protein encoding nucleic acid sequence operably linked to a constitutively active promoter.

51. The cancer cell line according to Clause 50, wherein the immune checkpoint protein encoding nucleic acid sequence encodes CD47.
52. The cancer cell line according to Clause 50, wherein the immune checkpoint protein encoding nucleic acid sequence encodes PD-L1.
53. The cancer cell line according to any of Clauses 50 to 52, wherein the immune checkpoint protein encoding nucleic acid sequence encodes CD47 and PD-L1.
54. The cancer cell line according to any of Clauses 50 to 53, wherein the transcriptional response element is a tetracycline-dependent promoter.
55. The cancer cell line according to any of Clauses 50 to 53, wherein the transcriptional response element is a GAL4 responsive upstream activating sequence (UAS).
56. The cancer cell line according to any of Clauses 50 to 55, wherein the inducible MYC locus further comprises a conditional genetic stop sequence between the transcriptional response element and the MYC encoding nucleic acid sequence.
57. The cancer cell line according to any of Clauses 50 to 56, wherein the cancer cell line is a lymphoma/leukemia cell line, a carcinoma cell line or a melanoma cell line.
58. The cancer cell line according Clause 57, wherein the lymphoma/leukemia cell line is an acute lymphoblastic leukemia cell line.
59. The cancer cell line according to Clause 57, wherein the carcinoma cell line is a hepatocellular carcinoma cell line, a non-small cell lung carcinoma cell line, a renal cell carcinoma cell line or a colorectal carcinoma cell line.
60. A transgenic non-human mammal comprising a cancer cell comprising:

(a) an inducible MYC locus comprising a MYC encoding nucleic acid sequence operably linked to a transcriptional response element; and

(b) a MYC-independent constitutively active locus comprising an immune checkpoint protein encoding nucleic acid sequence operably linked to a constitutively active promoter.

61. The transgenic non-human mammal according to Clause 60, wherein the transgenic non-human mammal is a rodent.
62. The transgenic non-human mammal according to Clause 61, wherein the rodent is a mouse.
63. The transgenic non-human mammal according to Clause 60, wherein the transgenic non-human mammal is a non-human primate.
64. The transgenic non-human mammal according to any of Clauses 60 to 63, wherein the immune checkpoint protein encoding nucleic acid sequence encodes CD47.
65. The transgenic non-human mammal according to any of Clauses 60 to 63, wherein the immune checkpoint protein encoding nucleic acid sequence encodes PD-L1.
66. The transgenic non-human mammal according to any of Clauses 60 to 65, wherein the immune checkpoint protein encoding nucleic acid sequence encodes CD47 and PD-L1.
67. The transgenic non-human mammal according to any of Clauses 60 to 66, wherein the transcriptional response element is a tetracycline-dependent promoter.
68. The transgenic non-human mammal according to any of Clauses 60 to 66, wherein the transcriptional response element is a GAL4 responsive upstream activating sequence (UAS).
69. The transgenic non-human mammal according to any of Clauses 60 to 68, wherein the inducible MYC locus further comprises a conditional genetic stop sequence between the transcriptional response element and the MYC encoding nucleic acid sequence.
70. The transgenic non-human mammal according to any of Clauses 60 to 69, wherein the cancer cell is a lymphoma/leukemia cell, a carcinoma cell or a melanoma line.
71. The transgenic non-human mammal according to Clause 70, wherein the lymphoma/leukemia cell is an acute lymphoblastic leukemia cell.
72. The transgenic non-human mammal according to Clause 70, wherein the carcinoma cell is a hepatocellular carcinoma cell, a non-small cell lung carcinoma cell, a renal cell carcinoma cell or a colorectal carcinoma cell.
73. A kit comprising:

a reagent for measuring the expression of MYC in a cellular sample from a subject; and

an immune checkpoint inhibitor.

74. The kit according to Clause 73, wherein the reagent for measuring the expression of MYC is a reagent that specifically binds MYC protein.
75. The kit according to Clause 74, wherein the reagent that specifically binds MYC protein is detectably labeled.
76. The kit according to Clauses 73 or 74, wherein the reagent that specifically binds MYC protein is an anti-MYC antibody.
77. The kit according to Clause 73, wherein the reagent for measuring the expression of MYC is a reagent that specifically binds a MYC encoding mRNA.
78. The kit according to Clause 76, wherein the reagent that specifically binds the MYC encoding mRNA is detectably labeled.
79. The kit according to Clauses 77 or 78, wherein the reagent that specifically binds the MYC encoding mRNA is a nucleic acid probe that specifically hybridizes to the MYC encoding mRNA.
80. The kit according to any of Clauses 73 to 79, wherein the immune checkpoint inhibitor is selected from the group consisting of: a PD-L1 inhibitor, a PD1 inhibitor, a CD47 inhibitor and a SIRPα inhibitor.
81. The kit according to any of Clause 73 to 80, wherein the kit comprises two or more immune checkpoint inhibitors.
82. The kit according to Clause 81, wherein the two or more immune checkpoint inhibitors comprise a first immune checkpoint inhibitor selected from a PD-L1 inhibitor or a PD1 inhibitor and a second immune checkpoint inhibitor selected from a CD47 inhibitor or a SIRPα inhibitor.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of identifying whether a subject having a cancer will be responsive to an immune checkpoint inhibitor, the method comprising:
(a) determining whether a cellular sample from the cancer overexpresses a MYC oncogene; and
(b) identifying the subject as (i) responsive to the immune checkpoint inhibitor when the determining indicates that the cancer overexpresses the MYC oncogene or (ii) as nonresponsive to the immune checkpoint inhibitor when the determining indicates that the cancer does not overexpress the MYC oncogene.

2. The method according to claim 1, wherein the immune checkpoint inhibitor is selected from the group consisting of: a CD47 inhibitor, a SIRPα inhibitor, a PD-L1 inhibitor and a PD1 inhibitor.

3. The method according to claim 1, wherein the method further comprises measuring the level of expression of the MYC oncogene in the cellular sample.

4. The method according to claim 3, wherein the level of expression comprises an mRNA expression level or a protein expression level.

5. The method according to claim 1, wherein the cancer is selected from the group consisting of: a lymphoma/leukemia, a carcinoma and a melanoma.

6. A method of treating a subject for a cancer, the method comprising:
(a) identifying whether the cancer will be responsive to an immune checkpoint inhibitor according to claim 1; and
(b) administering an effective amount of an agent to the subject to treat the cancer, wherein when the cancer is identified as responsive the agent is the immune checkpoint inhibitor.

7. A method of treating a subject for an immune-evasive cancer, the method comprising:
(a) determining whether a cellular sample of the immune-evasive cancer obtained from the subject overexpresses CD47, PD-L1 or both; and
(b) administering an effective amount of an agent to the subject to treat the immune-evasive cancer, wherein when the cellular sample is determined to overexpress CD47, PD-L1 or both the agent is a MYC inhibitor.

8. The method according to claim 7, wherein the MYC inhibitor is a bromodomain and extra-terminal (BET) inhibitor.

9. The method according to claim 8, wherein the BET inhibitor is JQ1 or an analog thereof.

10-12. (canceled)

13. A cancer cell comprising:
(a) an inducible MYC locus comprising a MYC encoding nucleic acid sequence operably linked to a transcriptional response element; and
(b) a MYC-independent constitutively active locus comprising an immune checkpoint protein encoding nucleic acid sequence operably linked to a constitutively active promoter.

14. A transgenic non-human mammal comprising the cancer cell according to claim 13.

15. A kit for performing the method according to claim 6, the kit comprising:
a reagent for measuring the expression of MYC in the cellular sample from the subject; and
the immune checkpoint inhibitor.

16. The cancer cell according to claim 13, wherein the immune checkpoint protein is CD47 or PD-L1.

17. A method of identifying a therapeutic agent that is effective during MYC-regulated immune evasion of a cancer cell, the method comprising:
(a) contacting (i) a first tumor cell line conditionally expressing a MYC oncogene and (ii) a second tumor cell line comprising the cancer cell according to claim 13 with a candidate therapeutic agent;
(b) determining the effect of the contacting on a behavior of the contacted conditional tumor cell lines;
(c) identifying the candidate agent as a therapeutic agent that is effective during MYC-regulated immune evasion of a cancer cell if the determined behavior of the first conditional tumor cell line (i) is reduced as compared to a non-contacted control and the determined behavior of the second conditional tumor cell line (ii) is not reduced as compared to a non-contacted control.

18. The method according to claim 17, wherein the behavior comprises cell line expansion or cell line survival.


SEQUENCE LISTING (40 sequences)

SEQ ID NO: 1 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 2 - protein, 439 aa, Mus musculus
MPLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 3 - protein, 453 aa, Rattus norvegicus
MNFLWEVENPTVTTMPLNVSFANRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSSSSDSLLSSESSPRATPEPLVLHEETPPTTSSDSEEEQDDEEEIDVVSVEKRQPPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSVQADEHKLISEKDLLRKRREQLKHKLEQLRNSGA

SEQ ID NO: 4 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 5 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 6 - protein, 178 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 7 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 8 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 9 - protein, 292 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 10 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 11 - protein, 311 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 12 - protein, 303 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 13 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 14 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 15 - protein, 508 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 16 - protein, 503 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 17 - protein, 54 aa, Artificial sequence
  synthetic peptide
  MISC_FEATURE (31)..(35): the amino acid residues at these positions may      be repeated 150 or 160 times
  MISC_FEATURE (34)..(34): the amino acid residue at this position may be      a V, G or A
MRRIRPRPPRLPRPRPRPLPFPRPGGCYPGVPGXGWPGSGNELKRAFAALRDQI

SEQ ID NO: 18 - protein, 28 aa, Artificial sequence
  synthetic peptide
QAETQKLISEIDLLRKQNEQLKHKLEQL

SEQ ID NO: 19 - protein, 90 aa, Artificial sequence
  synthetic peptide
TEENVKRRTHNVLERQRRNELKRSFFALRDQIPELENNEKAPKVVILKKATAYILSVQAETQKLISEIDLLRKQNEQLKHKLEQLRNSCA

SEQ ID NO: 20 - protein, 12 aa, Artificial sequence
  synthetic peptide
CERVIGTGWVRC

SEQ ID NO: 21 - DNA, 20 bp, Artificial sequence
  synthetic polynucleotide
ctgcgacgaggaggagaact

SEQ ID NO: 22 - DNA, 20 bp, Artificial sequence
  synthetic polynucleotide
ggcagcagctcgaatttctt

SEQ ID NO: 23 - DNA, 22 bp, Artificial sequence
  synthetic polynucleotide
agcagattttggatcagaccag

SEQ ID NO: 24 - DNA, 22 bp, Artificial sequence
  synthetic polynucleotide
gctccttcatggactgtagctg

SEQ ID NO: 25 - DNA, 22 bp, Artificial sequence
  synthetic polynucleotide
cggcttagttctctgtggagtc

SEQ ID NO: 26 - DNA, 22 bp, Artificial sequence
  synthetic polynucleotide
ggcatcagacacaagaggtagg

SEQ ID NO: 27 - DNA, 18 bp, Artificial sequence
  synthetic polynucleotide
tcttggatctccacaagc

SEQ ID NO: 28 - DNA, 19 bp, Artificial sequence
  synthetic polynucleotide
ctccaggtttcccatttac

SEQ ID NO: 29 - DNA, 20 bp, Artificial sequence
  synthetic polynucleotide
gtcgcaggttcttggtcact

SEQ ID NO: 30 - DNA, 19 bp, Artificial sequence
  synthetic polynucleotide
atcgcacgaacttcaccaa

SEQ ID NO: 31 - DNA, 21 bp, Artificial sequence
  synthetic polynucleotide
tgcggttcagctcaactactg

SEQ ID NO: 32 - DNA, 20 bp, Artificial sequence
  synthetic polynucleotide
gctttgcgcctccacattac

SEQ ID NO: 33 - DNA, 21 bp, Artificial sequence
  synthetic polynucleotide
gctccaaaggacttgtacgtg

SEQ ID NO: 34 - DNA, 21 bp, Artificial sequence
  synthetic polynucleotide
tgatctgaagggcagcatttc

SEQ ID NO: 35 - DNA, 20 bp, Artificial sequence
  synthetic polynucleotide
agcccagtgttaccaccaag

SEQ ID NO: 36 - DNA, 20 bp, Artificial sequence
  synthetic polynucleotide
acccaagaacaagcacaagg

SEQ ID NO: 37 - DNA, 20 bp, Artificial sequence
  synthetic polynucleotide
ggcaatgacgaaggaggtta

SEQ ID NO: 38 - DNA, 20 bp, Artificial sequence
  synthetic polynucleotide
atccggtggtatggatgaga

SEQ ID NO: 39 - DNA, 18 bp, Artificial sequence
  synthetic polynucleotide
ggcatttgctgaacgcat

SEQ ID NO: 40 - DNA, 18 bp, Artificial sequence
  synthetic polynucleotide
caattagtgcagccaggt